SEQUENCE LISTING (26 sequences)

SEQ ID NO: 1 - protein, 217 aa, synthetic construct
DIQMTQSPSSLSASVGDRVTITCQSIKSVYNNRLGWYQQKPGKAPKLLIYETSILTSGVPSRFSGSGSGTDFTLTISSLQPEDFATYYCAGGFDRSGDTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 230 aa, synthetic construct
EVQLVESGPGLVKPSETLSLTCTVSRFSLIGYAITWIRQPPGKGLEWIGGISSAATTFYSSWAKSRVTISVDTSKNQFSLKLSSVTAADTAVYYCARDPRGYGAALDRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 3 - protein, 217 aa, synthetic construct
DIQMTQSPSSLSASVGDRVTITCQSIKSVYNNRLGWYQQKPGKAPKLLIYETSILTSGVPSRFSGSGSGTDFTLTISSLQCEDFATYYCAGGFDRSGDTTFGQGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 4 - protein, 237 aa, synthetic construct
EVQLVESGPGCVKPSETLSLTCTVSRFSLIGYAITWIRQPPGKGLEWIGGISSAATTFYSSWAKSRVTISVDTSKNQFSLKLSSVTAADTAVYYCARDPRGYGAALDRLDLWGQGTLVTVSSASTKGPSVFPLAPSSKSTSGGTAALGCLVKDYFCEPVTVSWNSGALTSGVHTFPAVLQSSGLYSLSSVVTVPSSSLGTQTYICNVNHKPSNTKVDKKVEPKSCDKTHTHHHHHHP

SEQ ID NO: 5 - protein, 217 aa, synthetic construct
DIQMTQSPSSLSASVGDRVTITCQSIKSVYNNRLGWYQQKCGKAPKLLIYETSILTSGVPSRFSGSGSGTDFTLTISSLQCEDFATYYCAGGFDRSGDTTFGQGTKVEIKRTVAAPSVFIFPPSDEQLKSGTASVVCLLNNFYPREAKVQWKVDNALQSGNSQESVTCQDSKDCTYSLSSTLTLSKADYEKHKVYACEVTHQGLSSPVTKSFNRGEC

SEQ ID NO: 6 - protein, 238 aa, synthetic construct
EVQLVESGPGCVKPSETLSLTCTVSRFSLIGYAITWIRQPPGKGLEWIGGISSAATTFYSSWAKSRVTISVDTSKNQFSLKLSSVTAADTAVYYCARDPRGYGAALDRLDLWGQGTCVTVSSASTKGPSVFPLAPSSKSTSGGTAALGCLVKDYFCECPVTVSWNSGALTSGVHTFPAVLQSSGLYSLSSVVTVPSSSLGTQTYICNVNHKPSNTKVDKKVEPKSCDKTHTHHHHHHP

SEQ ID NO: 7 - protein, 217 aa, synthetic construct
DIQMTQSPSSLSASVGDRVTITCQSIKSVYNNRLGWYQQKCGKAPKLLIYETSILTSGVPSRFSGSGSGTDFTLTISSLQCEDFATYYCAGGFDRSGDTTFGQGTKVEIKRTVAAPSVCIFPPSDEQLKSGTASVVCLLNNFYPREAKVQWKVDNALQSGNSQESVTCQDSKDCTYSLSSTLTLSKADYEKHKVYACEVTHQGLSSPVTKSFNRGEC

SEQ ID NO: 8 - protein, 238 aa, synthetic construct
EVQLVESGPGCVKPSETLSLTCTVSRFSLIGYAITWIRQPPGKGLEWIGGISSAATTFYSSWAKSRVTISVDTSKNQFSLKLSSVTAADTAVYYCARDPRGYGAALDRLDLWGQGTCVTVSSASTKGPSVFPLAPSSKSTSGGTACLGCLVKDYFCECPVTVSWNSGALTSGVHTFPAVLQSSGLYSLSSVVTVPSSSLGTQTYICNVNHKPSNTKVDKKVEPKSCDKTHTHHHHHHP

SEQ ID NO: 9 - protein, 217 aa, synthetic construct
DIQMTQSPSSLSASVGDRVTITCQSIKSVYNNRLGWYQQKCGKAPKLLIYETSILTSGVPSRFSGSGSGTDFTLTISSLQCEDFATYYCAGGFDRSGDTTFGQGTKVEIKRTVAAPSVCIFPPSDECLKSGTASVVCLLNNFYPREAKVQWKVDNALQSGNSQESVTCQDSKDCTYSLSSTLTLSKADYEKHKVYACEVTHQGLSSPVTKSFNRGEC

SEQ ID NO: 10 - protein, 238 aa, synthetic construct
EVQLVESGPGCVKPSETLSLTCTVSRFSLIGYAITWIRQPPGKGLEWIGGISSAATTFYSSWAKSRVTISVDTSKNQFSLKLSSVTAADTAVYYCARDPRGYGAALDRLDLWGQGTCVTVSSASTKGPSVCPLAPSSKSTSGGTACLGCLVKDYFCECPVTVSWNSGALTSGVHTFPAVLQSSGLYSLSSVVTVPSSSLGTQTYICNVNHKPSNTKVDKKVEPKSCDKTHTHHHHHHP

SEQ ID NO: 11 - protein, 215 aa, synthetic construct
EIVLTQSPALMAASPGEKVTITCSVSLSISSSNLFWYQQKSETSPKPWIYGTSKLASGVPVRFSGSGSGTSYSLTISSMEAEDAATYYCQQWSSHSFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 228 aa, synthetic construct
EVQLVESGGGLVKPGGSLKLSCAASGFTFSNYAMSWVRQTPEKRLEWVATISNGGRYTYYPDSVKGRFTISRDNAKNSLYLQMSSLRSEDTAMYYCARHLYRYDVGGALDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKP

SEQ ID NO: 13 - protein, 215 aa, synthetic construct
EIVLTQSPALMAASPGEKVTITCSVSLSISSSNLFWYQQKCETSPKPWIYGTSKLASGVPVRFSGSGSGTSYSLTISSMECEDAATYYCQQWSSHSFTFGGGTKLEIKRADAAPTVSIFPPSSEQLTSGGASVVCFLNNFYPKDINVKWKIDGSERQNGVLNSWTCQDSKDCTYSMSSTLTLTKDEYERHNSYTCEATHKTSTSPIVKSFNRNEC

SEQ ID NO: 14 - protein, 236 aa, synthetic construct
EVQLVESGGGCVKPGGSLKLSCAASGFTFSNYAMSWVRQTPEKRLEWVATISNGGRYTYYPDSVKGRFTISRDNAKNSLYLQMSSLRSEDTAMYYCARHLYRYDVGGALDYWGQGTCVTVSSAKTTAPSVYPLAPVCGDTTGSSVTLGCLVKGYFCECPVTLTWNSGSLSSGVHTFPAVLQSDLYTLSSSVTVTSSTWPSQSITCNVAHPASSTKVDKKIEPRGPTIKPHHHHHHP

SEQ ID NO: 15 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 229 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 17 - protein, 213 aa, synthetic construct
QIVLSQSPAILSASPGEKVTMTCRASSSVSYIHWFQQKCGSSPKPWIYATSNLASGVPVRFSGSGSGTSYSLTISRVECEDAATYYCQQWTSNPPTFGGGTKLEIKRTVAAPSVFIFPPSDEQLKSGTASVVCLLNNFYPREAKVQWKVDNALQSGNSQESVTCQDSKDCTYSLSSTLTLSKADYEKHKVYACEVTHQGLSSPVTKSFNRGEC

SEQ ID NO: 18 - protein, 237 aa, synthetic construct
QVQLQQPGAECVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTCVTVSAASTKGPSVFPLAPSSKSTSGGTAALGCLVKDYFCECPVTVSWNSGALTSGVHTFPAVLQSSGLYSLSSVVTVPSSSLGTQTYICNVNHKPSNTKVDKKVEPKSCDKTHTHHHHHHP

SEQ ID NO: 19 - protein, 215 aa, synthetic construct
DIQMTQSPSSLSASVGDRVTITCRASQFLSSFGVAWYQQKPGKAPKLLIYGASSLYSGVPSRFSGSGSGTDFTLTISSLQPEDFATYYCQQGLLSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 228 aa, synthetic construct
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 21 - protein, 215 aa, synthetic construct
DIQMTQSPSSLSASVGDRVTITCRASQFLSSFGVAWYQQKCGKAPKLLIYGASSLYSGVPSRFSGSGSGTDFTLTISSLQCEDFATYYCQQGLLSPLTFGQGTKVEIKRTVAAPSVCIFPPSDECLKSGTASVVCLLNNFYPREAKVQWKVDNALQSGNSQESVTCQDSKDCTYSLSSTLTLSKADYEKHKVYACEVTHQGLSSPVTKSFNRGEC

SEQ ID NO: 22 - protein, 236 aa, synthetic construct
EVQLVESGGGCVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTCVTVSSASTKGPSVCPLAPSSKSTSGGTACLGCLVKDYFCECPVTVSWNSGALTSGVHTFPAVLQSSGLYSLSSVVTVPSSSLGTQTYICNVNHKPSNTKVDKKVEPKSCDKTHTHHHHHHP

SEQ ID NO: 23 - protein, 215 aa, synthetic construct
SVLTQPPSASGTPGQRVTISCSGSSSNIGSNYVYWYQQLPGTAPKLLIYRNNQRPSGVPDRFSGSKSGTSASLAISGLRSEDEADYYCAAWDERLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 229 aa, synthetic construct
EVQLQESGPGLVKPPGTLSLTCAVSGGSISSSNWWSWVRQPPGKGLEWIGEIYHSGSTNYNPSLKSRVTISVDKSKNQFSLKLSSVTAADTAVYYCARGSSSWYDLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDTHT

SEQ ID NO: 25 - protein, 216 aa, synthetic construct
SVLTQPPSASGTPGQRVTISCSGSSSNIGSNYVYWYQQLCGTAPKLLIYRNNQRPSGVPDRFSGSKSGTSASLAISGLRCEDEADYYCAAWDERLSGWVFGGGTKLTVLGQPKAAPSVTLFPPSSEELQANKATLVCLISDFYPGAVTVAWKADSSPVKAGVETTTPSCQSNCNKYAASSYLSLTPEQWKSHRSYSCQVTHEGSTVEKTVAPTECS

SEQ ID NO: 26 - protein, 238 aa, synthetic construct
EVQLQESGPGCVKPPGTLSLTCAVSGGSISSSNWWSWVRQPPGKGLEWIGEIYHSGSTNYNPSLKSRVTISVDKSKNQFSLKLSSVTAADTAVYYCARGSSSWYDLGPFDYWGQGTCVTVSSASTKGPSVFPLAPSSKSTSGGTAALGCLVKDYFCECPVTVSWNSGALTSGVHTFPAVLQSSGLYSLSSVVTVPSSSLGTQTYICNVNHKPSNTKVDKKVEPKSCDKTHTHHHHHHP

(19) United States
(12) Patent Application Publication
(10) Pub. No.: US 2026/0348974 A1
(43) Pub. Date: Nov. 5, 2026

(54) METHODS OF STRUCTURE DETERMINATION USING ANTIBODIES

(71) Applicant: Genentech, Inc., South San Francisco, CA (US)

(72) Inventors: Jennifer Elaine KUNG, San Francisco, CA (US); Jawahar SUDHAMSU, Pacifica, CA (US)

(21) Appl. No.: 19/703,479

(22) Filed: Jun. 10, 2026

Related U.S. Application Data
(63) Continuation of application No. PCT/US2024/060051, filed on Dec. 13, 2024.
(60) Provisional application No. 63/610,212, filed on Dec. 14, 2023.

(51) Int. Cl. G01N 33/68; G01N 21/84

(52) U.S. Cl. CPC G01N 33/6857; G01N 21/84

(57) ABSTRACT

Herein is provided a method of determining a high-resolution structure of a molecule comprising determining a high-resolution structure of a complex comprising the molecule and an antibody or antigen-binding fragment thereof or the antibody scaffold that binds to the molecule, wherein the antibody or antigen-binding fragment comprises one or more engineered disulfide bonds that increase conformational rigidity of the antibody or antigen-binding fragment thereof or the antibody scaffold, and thereby determining the high-resolution structure of the molecule using cryo-EM.

### CROSS-REFERENCE TO RELATED APPLICATIONS

This application is a continuation of International Application No. PCT/US2024/060051, filed internationally on Dec. 13, 2024, which claims the priority benefit of U.S. Provisional Patent Application Ser. No. 63/610,212, filed on Dec. 14, 2023, the disclosures of which are herein incorporated by reference in their entirety.

### REFERENCE TO AN ELECTRONIC SEQUENCE LISTING

The content of the electronic sequence listing (146392067501SeqList.xml; Size: 29,199 bytes; and Date of Creation: Jun. 8, 2026) is herein incorporated by reference in its entirety.

### FIELD OF INVENTION

Provided herein are methods of determining a high-resolution structure of a molecule comprising determining a high-resolution structure of a complex comprising the molecule and an antibody or antigen-binding fragment thereof or an antibody scaffold that binds to the molecule. Also provided herein are antibodies or antigen-binding fragments thereof comprising one or more engineered disulfide bonds that increase conformational rigidity of the antibody or antigen-binding fragment thereof or the antibody scaffold, and complexes comprising the antibody or antigen-binding fragment thereof or the antibody scaffold and a molecule.

### BACKGROUND OF THE INVENTION

Technological advances in cryo-electron microscopy (cryo-EM) have enabled the determination of high-resolution structures of large proteins and protein complexes. Solving the structures of small proteins (<100 kDa), however, remains a major challenge, as their lack of distinctive structural features and low signal-to-noise is prohibitive toward accurate image alignment and 3D reconstruction. Yet, most proteins in both prokaryotic and eukaryotic cells, including many drug targets, are smaller than 50 kDa. Yeates, T. O. et al., Curr Opin Struc Biol 60 (2020) 142-149. Consequently, this powerful technique remains inaccessible for a vast proportion of the proteome.

Several approaches have been proposed to address this problem. Most of these seek to increase the apparent size of the small protein by designing another protein, a “structure chaperone”, that binds with high affinity to the small protein. Structure chaperones, such as designed ankyrin repeat proteins (DARPins), nanobodies, or Fabs, can be evolved using in vivo and in vitro methods to bind tightly to specific targets. The small size (~15 kDa) of DARPins and nanobodies, however, limits their use as structure chaperones for cryo-EM. To overcome this limitation, both nanobodies and DARPins have been fused or bound to other proteins to produce elaborate scaffolds, such as “legobodies”, “pro-macrobodies”, “megabodies”, and self-assembling protein cages (Wu, X., et al., PNAS 118 (2021) e2115001118; Botte, M, et al., Nat Commun 13 (2022) 1826; Uchanski, T, et al., Nat Methods 18 (2021), 60-68; Liu, Y., et al., Nat commun 10 (2019) 1864; Castells-Graells, R., et al., Biorxiv doi:10,1101/2022.09.16.508009), of sufficient size to visualize a small protein of interest. However, inherent flexibility of the resulting fusion proteins and assemblies limits the overall resolution of the small target protein, which unfortunately ends up being the most poorly resolved part of the complex. (See, Liu, Y., et al., Nat commun 10 (2019) 1864; Yao, Q., et al., Structure (2019); Vulovic, I., et al., PNAS 118 (2021) e2015037118.)

Fabs, on the other hand, possess several favorable properties that make them an attractive option for structure chaperones: (a) novel Fabs can be discovered using well-established methods to bind any target with high affinity; (b) at ~50 kDa, Fabs by themselves are theoretically large enough to be resolved to high resolution via cryo-EM; (c) their distinctive shape provides a recognizable feature that facilitates accurate image alignment (Wu, S., et al, Structure 20 (2012) 582-592). The primary limitation of Fabs as structure chaperones for cryo-EM has been their inherent conformational flexibility (Stanfield, R. L., et al., J Mol Bio 357 (2006) 1566-1574).

High-resolution structures of proteins are critical to understanding molecular mechanisms of biological processes and in discovery of therapeutic molecules. Cryo-EM has revolutionized structure determination of large (>100 kDa) proteins and protein-protein complexes, but a vast majority of proteins that underlie human diseases are small (<50 kDa) and routinely out of reach for cryo-EM due to their lack of distinctive low-frequency structural features and the low signal-to-noise ratio in cryo-EM images, which prevent accurate image alignment.

Crystal structures of Fabs have revealed that there is a high degree of variability in the elbow angle between the variable and constant domains (115-225°) (Stanfield, R. L., et al., J Mol Bio 357 (2006) 1566-1574). This flexibility frequently causes the constant domain of the Fab to be poorly resolved in cryo-EM maps, and masks are often applied during refinement to exclude the Fab constant domain in order to achieve higher resolution at the Fab-antigen interface for large antigen-Fab complexes. Thus, only the variable domain of the Fab is truly utilized in these cases to aid in image alignment. If the Fab were conformationally rigid, however, the constant domain would (a) increase the overall size of the particle, (b) alleviate the need for a mask to discount the constant domain, and (c) improve image alignment. Previous attempts to generate conformationally rigid Fabs utilized phage display to shorten and mutate the heavy chain (HC) elbow of the Herceptin Fab framework, and clones were selected based on their thermal stability (Bailey, K. M., et al., J Mol Bio 430 (2018) 337-347. However, use of Fabs with these modifications in cryo-EM studies revealed that they are still quite flexible.

Therefore there remains a need in the art for methods of creating conformationally rigid Fabs that can be used for high resolution structure determination of small proteins as well as large proteins and protein complexes.

### SUMMARY OF THE INVENTION

Provided herein are methods of determining a high-resolution structure of a molecule comprising determining a high-resolution structure of a complex comprising the molecule and an antibody or antigen-binding fragment thereof or an antibody scaffold that binds to the molecule, the antibody or antigen-binding fragment or the antibody scaffold comprises one or more engineered disulfide bonds that increase conformational rigidity of the antibody or antigen-binding fragment thereof or the antibody scaffold, and thereby determining the high-resolution structure of the molecule.

In any of the proceeding embodiments, the complex comprising the molecule and the antibody or antigen-binding fragment thereof or the antibody scaffold is a non-covalent complex. In any of the proceeding embodiments, the complex comprising the molecule and the antibody or antigen-binding fragment thereof or the antibody scaffold is at least 40 kDa. In any of the proceeding embodiments, the antibody or antigen-binding fragment thereof or the antibody scaffold comprises a variable domain and a constant domain, and the one or more engineered disulfide bonds are between a cysteine located in the variable domain and a cysteine located in the constant domain.

Also provided herein are methods of increasing the rigidity of an antibody or antigen-binding fragment thereof or an antibody scaffold comprising a constant domain and a variable domain, the method comprising introducing a cysteine residue in the constant domain and introducing a cysteine residue in the variable domain, wherein the cysteine residue in the constant domain and the cysteine residue in the variable domain are sufficiently close in at least one conformation of the antibody or antigen-binding fragment thereof or the antibody scaffold to form an engineered disulfide bond, and allowing or inducing formation of the engineered disulfide bond, thereby increasing the rigidity of the antibody or antigen-binding fragment thereof or the antibody scaffold.

In some embodiments, the one or more engineered disulfide bonds reduce the variability in the elbow angle between the variable domain and the constant domain of the antibody or antigen-binding fragment thereof or the antibody scaffold. In some embodiments, the elbow angle of the antibody or antigen-binding fragment thereof or the antibody scaffold is between 137 and 159 degrees. In some embodiments, the elbow angle range is 5 degrees or less. In any of the proceeding embodiments, each of the one or more engineered disulfide bonds is between two amino acids whose C-beta atoms are positioned within 5.5 Å of each other in at least one conformation of the antibody or antigen-binding fragment thereof or the antibody scaffold. In some embodiments, each of the one or more engineered disulfide bonds is between two amino acids, and the C-beta atoms of said amino acids are positioned within 5.5 Å of each other in at least one conformation of the antibody or antigen-binding fragment thereof or the antibody scaffold. In any of the proceeding embodiments, the one or more engineered disulfide bonds are two, three, four, five, or six engineered disulfide bonds.

In any of the proceeding embodiments, the one or more engineered disulfide bonds comprise one disulfide bond between the heavy chain constant domain and the heavy chain variable domain and one disulfide bond between the light chain constant domain and the light chain variable domain. In any of the proceeding embodiments, the one or more engineered disulfide bonds comprise two disulfide bonds between the heavy chain constant domain and the heavy chain variable domain and two disulfide bonds between the light chain constant domain and the light chain variable domain. In any of the proceeding embodiments, the one or more engineered disulfide bonds comprise one or more disulfide bonds between the light chain constant domain and the heavy chain constant domain.

In any of the proceeding embodiments, the antibody or antigen-binding fragment thereof or the antibody scaffold is murine, rabbit, chimeric, humanized or human.

In some embodiments, the resolution of the high-resolution structure of the complex and/or the high-resolution structure of the molecule is determined to better than 4 Å, better than 3.5 Å, better than 3 Å or better than 2.8 Å.

In any of the proceeding embodiments, the one or more engineered disulfide bonds comprise residue pairs, according to Kabat numbering, 11^{HC}:151^{HC}, 108HC:152HC_153^{HC}insC, 40^{LC}:165^{LC}, 40^{LC}:166^{LC}, 80^{LC}:171^{LC}, 141^{HC}:116^{LC}, 126^{HC}:124^{LC}, 80^{LC}:170^{LC}_171^{LC}insC, 110^{HC}:151^{HC}, 106^{LC}:171^{LC}, 83^{LC}:166^{LC}, 81^{LC}:168^{LC}, 14^{HC}:113^{HC}, 14^{HC}:107^{HC}, 45^{HC}:44^{LC}, 183^{HC}:176^{LC} and/or 128^{HC}:118^{LC}.

In some embodiments, the one or more engineered disulfide bonds comprise 11^{HC}:151^{HC} and 80^{LC}:171^{LC}. In some embodiments, the one or more engineered disulfide bonds comprise 11^{HC}:151^{HC}, 108^{HC}:152^{HC}_153^{HC}insC, and 40^{LC}:165^{LC} and 80^{LC}:171^{LC}. In some embodiments, the one or more engineered disulfide bonds comprise 11^{HC}:151^{HC}, 108^{HC}:152^{HC}_153^{HC}insC, 40^{LC}:165^{LC}, 80^{LC}:171^{LC} and 141^{HC}: 16^{LC}. In some embodiments, the one or more engineered disulfide bonds comprise 11^{HC}:151^{HC}, 108^{HC}:152^{HC}_153^{HC}insC, 40^{LC}:165^{LC}, 80^{LC}:171^{LC}, 141^{HC}:16^{LC} and 126^{HC}:124^{LC}. In some embodiments, the one or more engineered disulfide bonds comprise 11^{HC}:151^{HC}, 108^{HC}:152^{HC}_153^{HC}insC, 40^{LC}:166^{LC} and 80^{LC}:170^{LC}_171^{LC}insC. In some embodiments, the one or more engineered disulfide bonds comprise 11^{HC}:151^{HC}, 108^{HC}:152^{HC}_153^{HC}insC, 40^{LC}: 166^{LC}, 80^{LC}:170^{LC}_17^{LC}insC, 141^{HC}:116^{LC}, and 126^{HC}:124^{LC}.

In any of the proceeding embodiments, the antibody or antigen-binding fragment thereof or the antibody scaffold comprises a kappa light chain or a lambda light chain. In some embodiments, the antibody or antigen-binding fragment thereof or the antibody scaffold comprises a kappa light chain. In some embodiments, the antibody or antigen-binding fragment thereof or the antibody scaffold comprises a lambda light chain.

In any of the proceeding embodiments, the molecule is a small molecule, peptide, protein, fusion protein, nucleic acid, or a lipid. In any of the proceeding embodiments, the molecule is a cytokine, a drug, or an enzyme substrate. In any of the proceeding embodiments, the molecule is less than 50 kDa, less than 30 kDa, or less than 10 kDa.

In some embodiments, determining the high-resolution structure comprises performing cryo-electron microscopy (cryo-EM) on the complex. In some embodiments, the method further comprises processing cryo-EM images. In some embodiments, the method further comprises performing refinement of an initial structure. In some embodiments, the method further comprises performing validation of a structure to determine the high-resolution structure. In some embodiments, the method further comprises generating the antibody or antigen-binding fragment thereof or the antibody scaffold that binds to the molecule, prior to determining the high-resolution structure. In some embodiments, the method further comprises incubating the antibody or antigen-binding fragment thereof or the antibody scaffold with the molecule to form a complex, prior to determining the high-resolution structure. In some embodiments, the method further comprises purifying the complex after the incubation.

In some embodiments, the method further comprises prior to determining the high-resolution structure, producing the antibody or antigen-binding fragment thereof or the antibody scaffold comprising one or more engineered disulfide bonds that increase rigidity of the antibody by (i) analyzing structural data from the antibody or antigen-binding fragment thereof or the antibody scaffold, or from an antibody or antigen-binding fragment thereof or the antibody scaffold obtained from the same species as the antibody or antigen-binding fragment thereof or the antibody scaffold; (ii) identifying amino acid residue positions in the constant domain and the variable domain that are sufficiently close to form a disulfide bond; and (iii) introducing a cysteine at the identified amino acid residue positions or inserting a cysteine before or after the identified amino acid residue positions.

In any of the proceeding embodiments, the method is performed in vitro.

In any of the proceeding embodiments, the antibody or antigen-binding fragment thereof or the antibody scaffold is an antibody. In any of the proceeding embodiments, the antibody or antigen-binding fragment thereof or the antibody scaffold is an antigen-binding antibody fragment. In some embodiments, the antigen-binding antibody fragment is a Fab. In some embodiments, the antibody or antigen-binding fragment thereof or the antibody scaffold is an antibody scaffold.

Also provided herein are antibodies or antigen-binding fragments thereof or antibody scaffolds comprising one or more engineered disulfide bonds that increase rigidity of the antibody or antigen-binding fragment thereof or the antibody scaffold. In some embodiments, the antibody or antigen-binding fragment thereof or the antibody scaffold comprises a variable domain and a constant domain, and the one or more engineered disulfide bonds are between a cysteine located in the variable domain and a cysteine located in the constant domain. In some embodiments, the one or more engineered disulfide bonds reduce the variability in the elbow angle between the variable domain and the constant domain of the antibody or antigen-binding fragment thereof or the antibody scaffold.

In some embodiments, the one or more engineered disulfide bonds are between two amino acids that are positioned within 5.5 Å of each other in at least one conformation. In some embodiments, the one or more engineered disulfide bonds are two, three, four, five, or six engineered disulfide bonds. In some embodiments, the one or more engineered disulfide bonds are comprised of one disulfide bond between the heavy chain constant domain and one disulfide bond between the light chain constant domain and the light chain variable domain. In some embodiments, the one or more engineered disulfides are comprised of two disulfide bonds between the heavy chain constant domain and two disulfide bonds between the light chain constant domain and the light chain variable domain. In some embodiments, the one or more engineered disulfide bonds comprises one or more disulfide bonds between the light chain constant domain and the heavy chain constant domain.

In some embodiments, the one or more engineered disulfide bonds comprise residue pairs, according to Kabat numbering, 11^{HC}:151^{HC}, 108^{HC}:152^{HC}_153^{HC}insC, 40^{LC}:165^{LC}, 40^{LC}:166^{LC}, 80^{LC}:171^{LC}, 141^{HC}:116^{LC}, 126^{HC}:124^{LC}, 80^{LC}:170^{LC}_171^{LC}insC, 110HC:151^{HC}, 106^{LC}:171^{LC}, 83^{LC}:166^{LC}, 81^{LC}:168^{LC}, 14^{HC}:113^{HC}, ^{HC}:107^{HC}, 45^{HC}:44^{LC}, 183^{HC}:176^{LC}, and/or 128^{HC}: 18^{LC}. In some embodiments, the one or more engineered disulfide bonds comprise 11^{HC}:151^{HC} and 80^{LC}:17^{LC}. In some embodiments, the one or more engineered disulfide bonds comprise 11^{HC}:151^{HC}, 108^{HC}:152^{HC}_153^{HC}insC, and 40^{LC}:165^{LC} and 80^{LC}:171^{LC}. In some embodiments, the one or more engineered disulfide bonds comprise 11^{HC}:151^{HC}, 108^{HC}:152^{HC}_153^{HC}insC, 40^{LC}:165^{LC}, 80^{LC}:171^{LC} and 141^{HC}:116^{LC}. In some embodiments, the one or more engineered disulfide bonds comprise 11^{HC}:151^{HC}, 108^{HC}:152^{HC}_153^{HC}insC, 40^{LC}:165^{LC}, 80^{LC}:171^{LC}, 141^{HC}:116^{LC} and 126^{HC}:124^{LC}. In some embodiments, the one or more engineered disulfide bonds comprise 11^{HC}:151^{HC}, 108^{HC}:152^{HC}_153^{HC}insC, 40^{LC}:166^{LC} and 80^{LC}:170^{LC}_171^{LC}insC. In some embodiments, the one or more engineered disulfide bonds comprise 11^{HC}:151^{HC}, 108^{HC}:152^{HC}_153^{HC}insC, 40^{LC}:166^{LC}, 80^{LC}:170^{LC}_171^{LC}insC, 141^{HC}:116^{LC} and 126^{HC}:124^{LC}.

In some embodiments, the antibody or antigen-binding fragment thereof or the antibody scaffold comprises a kappa light chain or a lambda light chain. In some embodiments, the antibody or antigen-binding fragment thereof or the antibody scaffold comprises a kappa light chain. In some embodiments, the antibody or antigen-binding fragment thereof or the antibody scaffold comprises a lambda light chain.

In some embodiments, the antibody or antigen-binding fragment thereof or the antibody scaffold is an antibody. In some embodiments, the antibody or antigen-binding fragment thereof or the antibody scaffold is an antigen-binding antibody fragment. In some embodiments, the antigen-binding antibody fragment is a Fab.

In some embodiments, the antibody or antigen-binding fragment thereof or the antibody scaffold is an antibody scaffold.

Also provided herein are complexes comprising the antibody or antigen-binding fragment thereof or the antibody scaffold of some embodiments herein and a molecule. In some embodiments, the complex comprising the molecule and the antibody or antigen-binding fragment thereof or the antibody scaffold is a non-covalent complex.

In some embodiments, the complex comprising the molecule and the antibody or antigen-binding fragment thereof or the antibody scaffold is at least 40 kDa. In some embodiments, the antibody or antigen-binding fragment thereof or the antibody scaffold is an antibody. In some embodiments, the antibody or antigen-binding fragment thereof or the antibody scaffold is an antigen-binding fragment of an antibody. In some embodiments, the antigen-binding fragment of an antibody is a Fab. In some embodiments, the antibody or antigen-binding fragment thereof or the antibody scaffold is an antibody scaffold.

### DESCRIPTION OF THE FIGURES

FIGS. 1A-1G depict the design of conformationally rigid engineered disulfide bonds. FIG. 1A, top, shows a VH-VL-based alignment illustrating the wide range of elbow angles adopted by selected human, murine, and rat Fabs (adapted from Stanfield et al, J Mol Biol., 357:1566-1574, 2006). Fab PDB IDs: 1BBD, 1FAB, 1DBA, 1PLG, 1NL0. Bottom, VH-VL-based alignment illustrating the narrow range of elbow angles adopted by rabbit Fabs. PDB IDs: 5CON, 4JO1, 6LDY, 6191, 5DUB. FIGS. 1B-D show residues mutated to introduce intrachain disulfides in the elbow region (FIGS. 1B and 1C), as well as interchain disulfides in the variable and constant domains (FIG. 1D) mapped onto the crystal structure of the E104.v1.WT Fab (PDB: 6VVU) shown in cartoon representation with the heavy chain in dark gray and light chain in light gray. Insets show zoomed-in views of pairs of mutations and the Cβ-Cβ distances (dashed lines) between these residues. Distances were measured in PyMOL. Fabs containing the following pairs of residues mutated to cysteines resulted in loss of expression: 14^{HC}:113^{HC}, 4^{LC}:107^{LC}, 45^{HC}:44^{LC}, 176^{HC}:183^{LC}. FIGS. 1E-G show electron density contoured at 1σ from crystal structures of E104.v1.4DS.S112F (FIG. 1E), E104.v1.4DS.A114F (FIG. 1F), and E104.v1.5DS.A114F (FIG. 1G), confirming proper formation of the engineered disulfides in these constructs.

FIGS. 2A-2F demonstrate the flexibility of previously published structure chaperones. FIG. 2A depicts a local resolution map of a tetrameric darpin-aldolase fusion bound to target protein GFP. The aldolase scaffold is much more rigid and resolved to a higher resolution than the DARPin or GFP, likely owing to flexibility of the linker between the DARPin and aldolase, as well as flexibility within the DARPin itself. FIG. 2B shows a local resolution map of one subunit of a dodecameric cage fused to a DARPin binding the target protein GFP. The resolution of GFP is much lower than for the cage proteins, likely due to flexibility in the linker between the cage subunit and DARPin, as well as flexibility of the DARPin itself. FIG. 2C shows local resolution maps of GFP (left) and KRAS (right) bound to a subunit of a DARPin-dodecameric cage fusion in which the DARPins are fused more rigidly to the cage than in FIG. 2C. The increase in rigidity is apparent in comparison with FIG. 2C, but there is still some loss of resolution in GFP and KRAS distal from the DARPin, indicating some remaining flexibility in the scaffold. FIG. 2D shows local resolution map of histamine receptor 2 (H2R) bound to a scaffold made up of an anti-H2R nanobody (Nb):anti-Nb Fab (NabFab):anti-Fab Nb complex. While the Nb:NabFab core of the scaffold appears rigid and well-resolved, the target H2R has lower resolution. FIG. 2E shows a local resolution map of mitochondrial uncoupling protein 1 (UCP1) bound to two pro-macrobodies, PMb65 and PMb71, consisting of Nbs fused to maltose binding protein (MBP) with a di-proline linker. For both pro-macrobodies, the Nb portion of the scaffold is better resolved than the MBP portion, as well as the target protein, indicating flexibility in the scaffold. FIG. 2F shows a local resolution map of a homodimer of V. cholerae Na⁺-dependent dicarboxylate transporter VcINDY bound to two copies of a Fab in which the heavy chain has been mutated to reduce elbow flexibility. Despite the “elbow-locking” mutations, the low resolution of the constant domains relative to the variable domains of the Fabs indicates that the Fab is still very flexible. Local resolution maps for (A-F) were calculated using Relion and the half-maps deposited for each structure in Electron Microscopy Data Bank (EMDB).

FIGS. 3A-3N show design and characterization of Rigid Fabs. FIG. 3A shows the design of Rigid Fabs. To engineer Fabs that are conformationally rigid, two (2DS) or four (4DS) intrachain disulfides were introduced in the elbow region of both the light (light gray) and heavy (dark gray) chains to restrict the elbow angle between the variable and constant domains. Two additional interchain disulfides were introduced in the constant domain to further reduce the flexibility of this domain, leading to a Rigid Fab design containing six engineered disulfides (6DS). FIG. 3B shows the crystal structure of E104.v1.6DS Fab. Center, cartoon representation of E104.v1.6DS (HC, dark gray; LC, light gray) with disulfides shown in spheres. Insets show electron density contoured at 1a for each of the engineered disulfides. FIGS. 3C-3F show the cryo-EM maps of tryptase complexes with WT (FIG. 3C), 2DS (FIG. 3D), 4DS (FIG. 3E), and 6DS (FIG. 3F) variants of the E104.v1 Fab colored by local resolution. FIGS. 3G-3N shows the EM density for selected map regions illustrating improvement in resolution with increasingly rigid constructs of E104.v1: (FIGS. 3G-3J) tryptase P84-S86 and (FIGS. 3K-3N) E104.v1 HC A31-A34 from the indicated tryptase-Fab structures.

FIGS. 4A-4C illustrate that Rigid Fab mutations do not affect antigen binding. FIG. 4A depicts representative biolayer interferometry (BLI) traces showing binding of a dilution series of the indicated E104.v1 Fab constructs to immobilized biotinylated tryptase. Sensorgrams were normalized to a reference well containing only buffer. The dashed line indicates the beginning of the dissociation step. FIG. 4B and FIG. 4C show the BLI equilibrium values from FIG. 4A plotted as a function of Fab concentration. Symbols and error bars represent average response±SEM.

FIGS. 5A-5Q show cryo-EM sample preparation and image processing for tryptase-E104.v1 Fab complexes. FIG. 5A shows the SEC elution profile and gel showing formation of tryptase-E104.v1.WT Fab complex. FIGS. 5B-5D show the image processing workflow (FIG. 5B), representative 2D classes (FIG. 5C), Fourier shell correlation (FSC) curve (FIG. 5D), for tryptase-E104.v1.WT dataset. FIG. 5E shows the SEC elution profile and gel showing formation of tryptase-E104.v1.2DS Fab complex. FIGS. 5F-5H show the image processing workflow (FIG. 5F) representative 2D classes (FIG. 5G), and FSC curve (FIG. 5H) for tryptase-E104.v1.2DS dataset. FIG. 5I shows SEC elution profile and gel showing formation of tryptase-E104.v1.4DS Fab complex. FIGS. 5J-L show the image processing workflow (FIG. 5J) representative 2D classes (FIG. 5K), and FSC curve (FIG. 5L) for tryptase-E104.v1.4DS dataset. FIG. 5M shows SEC elution profile and gel showing formation of tryptase-E104.v1.6DS Fab complex. FIGS. 5N-P show the image processing workflow (FIG. 5N), representative 2D classes (FIG. 5O), and FSC curve (FIG. 5P) for tryptase-E104.v1.6DS dataset. FIG. 5Q shows ResLog plot (FSC_{0.143}) comparing the WT, 2DS, 4DS, and 6DS datasets. fsc_noisesub was calculated for each reconstruction using the ResLog Analysis job in CryoSparc.

FIGS. 6A-6L show cryo-EM structures of Nav1.7-7A9 Fab and CD20-RTX Fab complexes. FIG. 6A-6B show composite cryo-EM maps of Nav1.7 complexes with (FIG. 6A) WT or (FIG. 6B) 4DS variants of the 7A9 Fab colored by local resolution. Dashed boxes indicate the constant domains of the Fabs. FIGS. 6C-6F show EM density for selected map regions illustrating improvements in resolution in the 7A9 HC (FIGS. 6C-6D) and LC (FIGS. 6E-6F) from the indicated structures. FIGS. 6G-6H show composite cryo-EM maps of CD20 complexes with (FIG. 6G) WT or (FIG. 6H) 4DS variants of the rituximab (RTX) Fab colored by local resolution. Dashed boxes indicate the constant domains of the Fabs. FIG. 6I-6L show EM density for selected map regions illustrating improvements in resolution in the RTX HC (FIG. 6I-6J) or LC (FIG. 6K-6L) from the indicated structures.

FIGS. 7A-7L show cryo-EM sample preparation and image processing for Nav-7A9 Fab complexes. FIG. 7A shows the SEC elution profile and gel showing formation of Nav1.7-7A9.WT Fab complex. FIGS. 7B-7E show representative 2D classes (FIG. 7B), image processing workflow (FIG. 7C), FSC curve (FIG. 7D) for consensus refinement, FSC curve for local refinement with mask around Fabs (FIG. 7E), and FSC curve for local refinement with mask around Nav1.7 following particle subtraction (FIG. 7F) for Nav1.7-7A9.WT dataset. FIG. 7G shows SEC elution profile and gel showing formation of Nav1.7-7A9.4DS Fab complex. FIG. 7H-L show representative 2D classes (FIG. 7H), image processing workflow (FIG. 7I), FSC curve for consensus refinement (FIG. 7J), FSC curve for local refinement with a mask around Fabs (FIG. 7K), FSC curve for local refinement with mask around Nav1.7 following particle subtraction (FIG. 7L) for Nav1.7-7A9.4DS dataset.

FIGS. 8A-8L show cryo-EM sample preparation and image processing for CD20-RTX Fab complexes. FIG. 8A. SEC elution profile and gel showing formation of CD20.RTX Fab complex. FIG. 8B-8I show representative 2D classes (FIG. 8B), image processing workflow (FIG. 8C), FSC curve for consensus refinement (FIG. 8D), FSC curve for local refinement with mask around CD20 and variable domains (FIG. 8E), FSC curve for local refinement with mask around CD20 (FIG. 8F) for CD20-RTX.WT dataset. FIG. 8G shows SEC elution profile and gel showing formation of CD20.RTX-4DS Fab complex. FIGS. 8H-L shows representative 2D classes (FIG. 8H), image processing workflow (FIG. 8I), FSC curve for consensus refinement (FIG. 8J), FSC curve for local refinement with mask around CD20 and variable domains (FIG. 8K), FSC curve for local refinement with mask around CD20 (FIG. 8L) for CD20-RTX.WT dataset.

FIGS. 9A-9D show cryo-EM sample preparation and image processing for Ang2-5A12.6DS Fab complex. FIG. 9A show the SEC elution profile and gel showing formation of Ang2-ProA-ProG-5A12.6DS Fab complex. FIGS. 9B-9D show representative 2D classes (FIG. 9B), image processing workflow (FIG. 9C), FSC curve (FIG. 9D) for Ang2-5A12.6DS dataset.

FIGS. 10A-10G show cryo-EM structure of Ang2-5A12.6DS Fab complex. FIG. 10A shows the cryo-EM map of Ang2-5A12.6DS Fab complex at a resolution of 2.7 Å with Ang2 in light, Fab HC in dark gray, and LC in white. FIG. 10B shows cryo-EM map of Ang2-5A12.6DS complex colored by local resolution. FIGS. 10C-10G shows the EM density for the entire Ang2-5A12.6DS complex (FIG. 10C) and selected map regions (FIGS. 10D-10G) illustrating high resolution features in Ang2, RTX LC, and RTX HC.

FIGS. 11A-11H show cryo-EM structure of KRAS^{G12C}-GNE-1952-2H11.4DS Fab complex. FIG. 11A shows the cryo-EM map of KRAS^{G12C}-GNE-1952-2H111.4DS Fab complex at a resolution of 2.8 Å with KRAS^{G12C} in light gray, GNE-1952 and GDP in dark gray, 2H11.6DS HC in dark gray, and LC in white. FIG. 11B show cryo-EM map of KRAS^{G12C}-GNE-1952-2H11.4DS complex colored by local resolution. FIG. 11C shows the locally refined cryo-EM map of KRAS^{G12C}-GNE-1952-2H111.4DS complex colored by local resolution. FIGS. 11D-11H shows the EM density for the at the KRAS-Fab interface in the KRAS^{G12C}-GNE-1952-2H111.4DS complex (FIG. 11D), GNE-1952 covalently bound to C12 (FIG. 11E), GDP (FIG. 11F), and selected regions of KRAS^{G12C} (FIGS. 11G-11H) illustrating high resolution features.

FIGS. 12A-12F shows cryo-EM sample preparation and image processing for KRAS^{G12C}-GNE-1952-2H11.4DS Fab complex. FIG. 12A shows the design of LC elbow disulfide mutations for 2H11 Fab. Mutated residues are mapped onto a cartoon representation of 2H11^{LC} (light gray) from a crystal structure of a KRAS^{G12C}-2H11 crystal structure (PDB:7RP2), with Cβ-Cβ distances shown in dashed lines. Since P80 and N171 are too far to enable disulfide mutation by mutating both these sites to cysteine, a cysteine was inserted between N170 and N171. The other LC elbow disulfide was formed using cysteine mutations at P40 and K166. FIG. 12B shows the SEC elution profile and gel showing formation of KRAS^{G12C}-GNE-1952-2H11.4DS Fab complex. FIGS. 12C-12F show representative 2D classes (FIG. 12C), image processing workflow (FIG. 12D), FSC curve for consensus refinement (FIG. 12E), FSC curve for local refinement with mask around KRAS and the Fab variable domain (FIG. 12F) for the KRAS^{G12C}-GNE-1952-2H11.4DS dataset.

FIGS. 13A-13C show rigid Fab sequence and structural alignments. FIGS. 13A-13B show sequence alignments for the wild type (WT) and Rigid Fab variants of the light chains (FIG. 13A) and heavy chains (FIG. 13B) for the anti-tryptase Fab E104.v1, anti-Nav1.7 Fab 7A9, anti-CD20 Fab Rituximab (RTX), anti-Ang2 Fab 5A12, and anti-KRAS Fab 2H11. Positions of cysteine mutations introduced in the Rigid Fab 2DS, 4DS, and 6DS designs are denoted by stars, circles, and squares above the sequences, respectively. FIG. 13C shows positions mutated in the lambda LC of 2H11 are denoted by open stars and circles. VH-VL-based alignment illustrating the narrow range of elbow angles adopted by Rigid Fabs in the cryo-EM structures from this study. Elbow angles were calculated using http://linum.proteinmodel.org/AS2TS/RBOW/index.html.

### DETAILED DESCRIPTION OF THE INVENTION

Small proteins (<50 kDa) encompass a vast majority of all known proteins across living organisms, especially in the context of drug targets in humans, pathogens, etc. While many of these proteins are amenable to high resolution structure determination by x-ray crystallography, many others have had limited success due to low expression, low solubility, or lack of crystallizability. Advances in cryo-EM can circumvent the above challenges as the protein amount and concentration requirements are low. Yet, this method is currently limited mainly to large proteins and protein complexes, as small proteins are plagued by low signal-to-noise and difficulties in particle alignment.

Provided herein are methods of structural determination that employ Fabs with reduced flexibility by introducing cysteine substitutions in residue pairs in strategic positions to allow formation of new disulfide bonds, thereby restricting the range of possible elbow angles between the variable and constant domains. These Rigid Fabs allow high resolution structure determination of these small proteins using cryogenic electron microscopy (cryo-EM) by increasing the effective size of the target protein in a rigid manner and thereby improving particle alignment and pose assignments.

### I. DEFINITIONS

The term “amino acid” denotes the group of carboxy α-amino acids, either occurring naturally, i.e. which directly or in form of a precursor can be encoded by a nucleic acid, or occurring non-naturally. The individual naturally occurring amino acids are encoded by nucleic acids consisting of three nucleotides, so called codons or base-triplets. Each amino acid is encoded by at least one codon. This is known as “degeneration of the genetic code”. The term “amino acid” as used within this application denotes the naturally occurring carboxy α-amino acids comprising alanine (three letter code: ala, one letter code: A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). Examples of non-naturally occurring amino acids include, but are not limited to, Aad (alpha-aminoadipic acid), Abu (aminobutyric acid), Ach (alpha-aminocyclohexane-carboxylic acid), Acp (alpha-aminocyclopentane-carboxylic acid), Acpc (1-Aminocyclopropane-1-carboxylic acid), Aib (alpha-aminoisobutyric acid), Aic (2-Aminoindane-2-carboxylic acid; also called 2-2-Aic), 1-1-Aic (1-aminoindane-1-carboxylic acid), (2-aminoindane-2-carboxylic acid), allylglycine (allylGly), alloisoleucine (allo-Ile), Asu (alpha-aminosuberic acid, 2-aminooctanedioc acid), Bip (4-phenyl-phenylalanine-carboxylic acid), BnHP ((2S,4R)-4-hydroxyproline), Cha (beta-cyclohexylalanine), Cit (citrulline), cyclohexylglycine (Chg), cyclopentylalanine, beta-cyclopropyl alanine, Dab (1,4-Diaminobutyric acid), Dap (1,3-Diaminopropionic acid), p (3,3-diphenylalanine-carboxylic acid), 3,3-Diphenylalanine, Di-n-propylglycine (Dpg), 2-Furylalanine, Homocyclohexylalanine (HoCha), Homocitrulline (HoCit), Homocycloleucine, Homoleucin (HoLeu), Homoarginine (HoArg), Homoserine (HoSer), Hydroxyproline, Lys(Ac), (1) Nal (1-Naphtyl Alanine), (2) Nal (2-Naphtyl Alanine), 4-MeO-Apc (1-amino-4-(4-methoxyphenyl)-cyclohexane-1-carboxylic acid), Nor-leucine (Nle), Nva (Norvaline), Omathine, 3-Pal (alpha-amino-3-pyridylalanine-carboxylic acid), 4-Pal (alpha-amino-4-pyridylalanine-carboxylic acid), 3,4,5,F3-Phe (3,4,5-Trifluoro-phenylalanine), 2,3,4,5,6,F5-Phe (2,3,4,5,6-Pentafluoro-phenylalanine), Pqa (4-oxo-6-(1-piperazinyl)-3(4H)-quinazoline-acetic acid (CAS 889958-08-1)), Pyridylalanine, Quinolylalanine, Sarcosine (Sar), Thiazolylalanine, Thienylalanine, Tic (alpha-amino-1,2,3,4,tetrahydroisoquinoline-3-carboxylic acid), Tic(OH), Tle (tertbutylGlycine), and Tyr(Me).

The term “amino acid sequence variant” refers to polypeptides having amino acid sequences that differ to some extent from a native sequence polypeptide. Ordinarily, amino acid sequence variants will possess at least about 70% sequence identity with the native sequence polypeptide. In one embodiment the variant has about 80% or more sequence identity with the native sequence polypeptide. In one embodiment the variant has about 90% or more sequence identity with the native sequence polypeptide. In one embodiment the variant has about 95% or more sequence identity with the native sequence polypeptide. In one embodiment the variant has about 98% or more sequence identity with the native sequence polypeptide. The amino acid sequence variants possess substitutions, deletions, and/or insertions at certain positions within the amino acid sequence of the native amino acid sequence. Amino acids are designated by the conventional names, one-letter and three-letter codes.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “antibody fragment” denotes a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “binding site” or “antigen-binding site” as used herein denotes the region(s) of an antibody molecule to which a ligand (e.g. the antigen or antigen fragment of it) actually binds and which is derived from an antibody. The antigen-binding site includes antibody heavy chain variable domains (VH) and/or an antibody light chain variable domain (VL), or pairs of VH/VL.

The antigen-binding sites that specifically bind to the desired antigen can be derived a) from known antibodies to the antigen or b) from new antibodies or antibody fragments obtained by de novo immunization methods using inter alia either the antigen, protein, or nucleic acid or fragments thereof, or by phage display.

An antigen-binding site of an antibody of the invention can contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding. In some cases, a VH or a VL domain will be sufficient.

The term “bispecific antibodies” denotes antibodies which have two different binding specificities. In one embodiment bispecific antibodies as provided herein are specific for two different antigens.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called a, t, F, y, and p, respectively.

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “effector functions” denotes those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “effective amount” of an agent, e.g., a pharmaceutical formulation, denotes an amount effective, at dosages and for periods of time necessary, to achieve the desired therapeutic or prophylactic result.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242.

The term “framework”, short “FR”, denotes heavy and light chain variable domain amino acid residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “free cysteine amino acid” denotes a cysteine amino acid residue which has been engineered into a parent antibody, has a thiol functional group (SH), and is not paired as an intramolecular disulfide bridge. Nevertheless, a free cysteine amino acid can be paired as an intramolecular disulfide bridge, e.g. with glutathione.

The term “full length antibody” denotes an antibody having a structure substantially similar to a native antibody structure or having heavy chains that contain an Fc-region as defined herein. Native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

A “full length antibody” is an antibody comprising a VL and VH domain, as well as a light chain constant domain (CL) and heavy chain constant domains, CH1, CH2 and CH3. The constant domains may be native sequence constant domains (e.g., human native sequence constant domains) or an amino acid sequence variant thereof. The full length antibody may have one or more “effector functions” which refer to those biological activities attributable to the Fc constant region (a native sequence Fc-region or amino acid sequence variant Fc-region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B-cell receptor and BCR.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chrom. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “monospecific antibody” denotes an antibody that has one or more binding sites each of which has the same binding specificity, i.e. binds to the same antigen or motif amino acid sequence.

A “naked antibody” refers to an antibody that is not conjugated to a heterologous moiety (e.g., a cytotoxic moiety) or radiolabel. The naked antibody may be present in a pharmaceutical formulation.

A “parent antibody” is an antibody comprising an amino acid sequence from which one or more amino acid residues are replaced by one or more cysteine residues. The parent antibody may comprise a native or wild-type sequence. The parent antibody may have pre-existing amino acid sequence modifications (such as additions, deletions and/or substitutions) relative to other native, wild-type, or modified forms of an antibody.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-a and -P; mullerian-inhibiting substance; mouse gonadotropin-associated peptide; inhibin; activin; vascular endothelial growth factor; integrin; thrombopoietin (TPO); nerve growth factors such as NGF-p; platelet growth factor; transforming growth factors (TGFs) such as TGF-a and TGF-p; insulin-like growth factor-I and -II; erythropoietin (EPO); osteoinductive factors; interferons such as interferon-a, -P, and -y; colony stimulating factors (CSFs) such as macrophage-CSF (M-CSF); granulocyte-macrophage-CSF (GM-CSF); and granulocyte-CSF (GCSF); interleukins (ILs) such as IL-I, IL-1a, IL-2, IL-3, IL-4, IL-5, IL-6, IL-7, IL-8, IL-9, IL-IO, IL-II, IL-12; a tumor necrosis factor such as TNF-□ or TNF-P; and other polypeptide factors including LIF and kit ligand (KL). As used herein, the term cytokine includes proteins from natural sources or from recombinant cell culture and biologically active equivalents of the native sequence cytokines.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as, but not limited to, carbohydrate groups, metal ions, phosphate groups, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

All polypeptide sequences are written according to the generally accepted convention whereby the alpha-N-terminal amino acid residue is on the left and the alpha-C-terminal amino acid residue is on the right. As used herein, the term “N-terminus” refers to the free alpha-amino group of an amino acid in a polypeptide, and the term “C-terminus” refers to the free a-carboxylic acid terminus of an amino acid in a polypeptide. A polypeptide which is N-terminated with a group refers to a polypeptide bearing a group on the alpha-amino nitrogen of the N-terminal amino acid residue. An amino acid which is N-terminated with a group refers to an amino acid bearing a group on the alpha-amino nitrogen.

Unless indicated otherwise by a “D” prefix, e.g., D-Ala or N-Me-D-Ile, or written in lower case format, e.g., a, i, 1, (D versions of Ala, Ile, Leu), the stereochemistry of the alpha-carbon of the amino acids and aminoacyl residues in polypeptides described in this specification and the appended claims is the natural or “L” configuration. The Cahn-Ingold-Prelog “R” and “S” designations are used to specify the stereochemistry of chiral centers in certain acyl substituents at the N-terminus of the polypeptides. The designation “R,S” is meant to indicate a racemic mixture of the two enantiomeric forms. This nomenclature follows that described in Cahn, R. S., et al., Angew. Chem. Int. Ed. Engl. 5 (1966) 385-415.

The term “single-chain Fv”, short “scFv”, denotes an antibody fragment that comprises the VH and VL domains of antibody, wherein these domains are present in a single polypeptide chain. In one embodiment, the Fv polypeptide further comprises a polypeptide linker between the VH and VL domains which enables the scFv to form the desired structure for antigen binding. For a review of scFv, see Plueckthun in The Pharmacology of Monoclonal Antibodies, vol. 113, Rosenburg and Moore (Eds), Springer-Verlag, New York, pp. 269-315 (1994).

The term “x-valent”, e.g. “mono-valent” or “bi-valent” or “tri-valent” or “tetra-valent”, denotes the presence of a specified number of binding sites, i.e. “x”, in an antibody molecule. As such, the terms “bivalent”, “tetravalent”, and “hexavalent” denote the presence of two binding site, four binding sites, and six binding sites, respectively, in an antibody molecule. The bispecific antibodies as provided herein are at least “bivalent” and may be “trivalent” or “multivalent” (e.g. “tetravalent” or “hexavalent”). In one embodiment the bispecific antibody as provided herein is bivalent, trivalent, or tetravalent. In one embodiment the bispecific antibody is bivalent. In one embodiment the bispecific antibody is trivalent. In one embodiment the bispecific antibody is tetravalent.

In certain aspects and embodiments the antibodies as provided herein have two or more binding sites and are bispecific. That is, the antibodies may be bispecific even in cases where there are more than two binding sites (i.e. that the antibody is trivalent or multivalent). The term bispecific antibodies includes, for example, multivalent single chain antibodies, diabodies and triabodies, as well as antibodies having the constant domain structure of full length antibodies to which further antigen-binding sites (e.g., single chain Fv, a VH domain and/or a VL domain, Fab, or (Fab)2) are linked via one or more peptide-linkers. The antibodies can be full length from a single species, or be chimerized or humanized. For an antibody with more than two antigen binding sites, some binding sites may be identical, so long as the protein has binding sites for two different antigens.

The term “variable region” denotes the domain of an antibody heavy or light chain that is involved in binding the antibody to its antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt, T. J. et al. Kuby Immunology, 6th ed., W. H. Freeman and Co., N.Y. (2007), page 91) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628).

The term “vector” denotes a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. ANTIBODY OR ANTIGEN-BINDING FRAGMENT THEREOF OR ANTIBODY SCAFFOLD

### A. Antibodies

The invention relates to antibody or antigen-binding fragment thereof or the antibody scaffold comprising one or more engineered disulfide bonds that increase conformational rigidity of the antibody or antigen-binding fragment thereof or the antibody scaffold. In some embodiments, the antibody or antigen-binding fragment thereof or the antibody scaffold is an antibody. In some embodiments, the antibody or antigen-binding fragment thereof or the antibody scaffold is an antigen-binding fragment of an antibody. In some embodiments, the antigen-binding fragment of an antibody is a Fab. In some embodiments, the antigen-binding antibody fragment is a Fab. In some embodiments, the antibody or antigen-binding fragment thereof or the antibody scaffold is an antibody scaffold.

In some embodiments, the antibody is comprised of two heavy and two light chains. The light chain is classified as either a kappa (κ) or lambda (λ) chain based on small differences in the polypeptide sequence. The heavy chain defines the class or isotype of an antibody. Each component chain contains one NH2-terminal variable domain and one or more COOH-terminal constant domains. Each variable or constant domain consists of approximately 110-130 amino acids. Both light chains contain only one constant domain, whereas the heavy chains contain either three or four constant domains, which define the isotype. Heavy chains with three constant domains tend to include a spacer hinge region between the first and second constant domains. A typical light chain will have a mass of approximately 25 kDa and a three constant domain heavy chain with its hinge will have a mass of approximately 55 kDa.

In some embodiments, the antigen-binding fragment thereof is a Fab fragment. A Fab fragment contains the constant domain of the light chain and the first constant domain (CH1) of the heavy chain.

The “light chain of an antibody” as used herein is a polypeptide comprising in N-terminal to C-terminal direction an antibody light chain variable domain (VL), and an antibody light chain constant domain (CL), abbreviated as VL-CL.

The “heavy chain of an antibody” as used herein is a polypeptide comprising in N-terminal to C-terminal direction an antibody heavy chain variable domain (VH) and an antibody constant heavy chain domain 1 (CH1).

There are five major classes of antibodies: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called u, t, F, y, and p, respectively. In some embodiments, the antibody or antigen-binding fragment thereof or the antibody scaffold is an IgA. In some embodiments, the antibody or antigen-binding fragment thereof or the antibody scaffold is an IgD. In some embodiments, the antibody or antigen-binding fragment thereof or the antibody scaffold is an IgE. In some embodiments, the antibody or antigen-binding fragment thereof or the antibody scaffold is an IgG. In some embodiments, the antibody or antigen-binding fragment thereof or the antibody scaffold is an IgM.

In some embodiments, the molecular weight of the antibody or antibody fragment thereof is between about 40 kDa to about 150 kDa. In some embodiments, the antibody or antibody fragment thereof is a Fab and the molecular weight of the Fab is about 50 kDa. In some embodiments, the antibody or antibody fragment thereof is an IgG antibody, and the molecular weight of the IgG antibody is about 150 kDa.

In some embodiments, the antibody or antibody fragment thereof is generated by a method comprising, e.g., molecular cloning or a synthetic biology method known in the art, e.g., gene synthesis. In some embodiments, the antibody is generated by a method comprising immunizing a mouse, rabbit, or other appropriate mammal known in the art. In some embodiments, the antibody is generated by a method comprising construction of a phage display library. In some embodiments, the antibody or antibody fragment thereof is generated by a method comprising de novo immunization methods using inter alia either the antigen protein or nucleic acid or fragments thereof, or by phage display. In some embodiments, the antibody or antigen-binding fragment thereof or the antibody scaffold is generated by a method comprising expressing a construct of the antibody or antigen-binding fragment thereof or the antibody scaffold in Chinese Hamster Ovary cells. In some embodiments, the method further comprises purifying the antibody or antigen-binding fragment thereof or the antibody scaffold by one or more forms of chromatography (e.g., nickel affinity chromatography followed by size exclusion chromatography).

In some embodiments, the antibody or antigen-binding fragment thereof or the antibody scaffold is an antibody scaffold.

### Antibody Fragment

In some embodiments, the antibody or antigen-binding fragment thereof or the antibody scaffold is an antibody fragment. Antibody fragments include, but are not limited to, Fab, Fab′, Fab′-SH, F(ab′)₂, Fv, and scFv fragments, and other fragments described below. For a review of certain antibody fragments, see Hudson, P. J. et al., Nat. Med. 9 (2003) 129-134. For a review of scFv fragments, see, e.g., Plueckthun, A., In; The Pharmacology of Monoclonal Antibodies, Vol. 113, Rosenburg and Moore (eds.), Springer-Verlag, New York (1994), pp. 269-315; see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 0 404 097; WO 93/01161; Hudson, P. J. et al., Nat. Med. 9 (2003) 129-134; and Holliger, P. et al., Proc. Natl. Acad. Sci. USA 90 (1993) 6444-6448. Triabodies and tetrabodies are also described in Hudson, P. J. et al., Nat. Med. 9 (20039 129-134).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516).

Antibody fragments can be made by various techniques, including but not limited to proteolytic digestion of an intact antibody as well as production by recombinant host cells (e.g. E. coli or phage), as described herein.

### Chimeric and Humanized Antibodies

In certain embodiments, the antibody or antigen-binding fragment thereof or the antibody scaffold is a chimeric antibody. Certain chimeric antibodies are described, e.g., in U.S. Pat. No. 4,816,567; and Morrison, S. L. et al., Proc. Natl. Acad. Sci. USA 81 (1984) 6851-6855). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro, J. C. and Fransson, J., Front. Biosci. 13 (2008) 1619-1633, and are further described, e.g., in Riechmann, I. et al., Nature 332 (1988) 323-329; Queen, C. et al., Proc. Natl. Acad. Sci. USA 86 (1989) 10029-10033; U.S. Pat. Nos. 5,821,337, 7,527,791, 6,982,321, and 7,087,409; Kashmiri, S. V. et al., Methods 36 (2005) 25-34 (describing SDR (a-CDR) grafting); Padlan, E. A., Mol. Immunol. 28 (1991) 489-498 (describing “resurfacing”); Dall'Acqua, W. F. et al., Methods 36 (2005) 43-60 (describing “FR shuffling”); and Osbourn, J. et al., Methods 36 (2005) 61-68 and Klimka, A. et al., Br. J. Cancer 83 (2000) 252-260 (describing the “guided selection” approach to FR shuffling).

Human framework regions that may be used for humanization include but are not limited to: framework regions selected using the “best-fit” method (see, e.g., Sims, M. J. et al., J. Immunol. 151 (1993) 2296-2308; framework regions derived from the consensus sequence of human antibodies of a particular subgroup of light or heavy chain variable regions (see, e.g., Carter, P. et al., Proc. Natl. Acad. Sci. USA 89 (1992) 4285-4289; and Presta, L. G. et al., J. Immunol. 151 (1993) 2623-2632); human mature (somatically mutated) framework regions or human germline framework regions (see, e.g., Almagro, J. C. and Fransson, J., Front. Biosci. 13 (2008) 1619-1633); and framework regions derived from screening FR libraries (see, e.g., Baca, M. et al., J. Biol. Chem. 272 (1997) 10678-10684 and Rosok, M. J. et al., J. Biol. Chem. 271 (19969 22611-22618).

### Library-Derived Antibodies

In some embodiments, the antibody or antigen-binding fragment thereof or the antibody scaffold is isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom, H. R. et al., Methods in Molecular Biology 178 (2001) 1-37 and further described, e.g., in the McCafferty, J. et al., Nature 348 (1990) 552-554; Clackson, T. et al., Nature 352 (1991) 624-628; Marks, J. D. et al., J. Mol. Biol. 222 (1992) 581-597; Marks, J. D. and Bradbury, A., Methods in Molecular Biology 248 (2003) 161-175; Sidhu, S. S. et al., J. Mol. Biol. 338 (2004) 299-310; Lee, C. V. et al., J. Mol. Biol. 340 (2004) 1073-1093; Fellouse, F. A., Proc. Natl. Acad. Sci. USA 101 (2004) 12467-12472; and Lee, C. V. et al., J. Immunol. Methods 284 (2004) 119-132.

In certain phage display methods, repertoires of VH and VL genes are separately cloned by polymerase chain reaction (PCR) and recombined randomly in phage libraries, which can then be screened for antigen-binding phage as described in Winter, G. et al., Ann. Rev. Immunol. 12 (1994) 433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths, A. D. et al., EMBO J. 12 (1993) 725-734. Finally, naive libraries can also be made synthetically by cloning non-rearranged V-gene segments from stem cells, and using PCR primers containing random sequence to encode the highly variable CDR3 regions and to accomplish rearrangement in vitro, as described by Hoogenboom, H. R. and Winter, G., J. Mol. Biol. 227 (1992) 381-388. Patent publications describing human antibody phage libraries include, for example: U.S. Pat. No. 5,750,373, US 2005/0079574, US 2005/0119455, US 2005/0266000, US 2007/0117126, US 2007/0160598, US 2007/0237764, US 2007/0292936, and US 2009/0002360.

Antibodies or antibody fragments isolated from human antibody libraries are considered human antibodies or human antibody fragments herein.

### Antibody Formats

The antibody or antigen-binding fragment thereof or the antibody scaffold can be combined in multiple ways to generate different antibody formats.

For example, one or more scFv antibody fragments can be fused to the C-terminus of one or more polypeptide chains of a complete antibody. Especially to each heavy chain C-terminus or to each light chain C-terminus a scFv antibody fragment can be fused.

For example, one or more antibody Fab fragments can be fused to the C-terminus of one or more polypeptide chains of a complete antibody. Especially to each heavy chain C-terminus or to each light chain C-terminus an antibody Fab fragment can be fused.

For example, one scFv and one antibody Fab fragment can be fused to the N-termini of an antibody Fc-region.

For example one scFv or antibody Fab fragment can be fused to an N-terminus of an antibody Fc-region and one scFv or antibody Fab fragment can be fused to the C-terminus of the respective other chain of an antibody Fc-region.

### Multispecific Antibodies

A wide variety of recombinant antibody formats have been developed, e.g. tetravalent bispecific antibodies by fusion of, e.g., an IgG antibody format and single chain domains (see e.g. Coloma, M. J., et al., Nature Biotech 15 (1997) 159-163; WO 01/077342; and Morrison, S. L., Nature Biotech 25 (2007) 1233-1234).

Also several other formats wherein the antibody core structure (IgA, IgD, IgE, IgG or IgM) is no longer retained such as dia-, tria- or tetrabodies, minibodies, several single chain formats (scFv, Bis-scFv), which are capable of binding two or more antigens, have been developed (Holliger, P., et al., Nature Biotech 23 (2005) 1126-1136; Fischer, N., L6ger, O., Pathobiology 74 (2007) 3-14; Shen, J., et al., Journal of Immunological Methods 318 (2007) 65-74; Wu, C., et al., Nature Biotech. 25 (2007) 1290-1297).

All such formats use linkers either to fuse the antibody core (IgA, IgD, IgE, IgG or IgM) to a further binding protein (e.g. scFv) or to fuse e.g. two Fab fragments or scFvs (Fischer, N. and L6ger, O., Pathobiology 74 (2007) 3-14). It has to be kept in mind that one may want to retain effector functions, such as e.g. complement-dependent cytotoxicity (CDC) or antibody dependent cellular cytotoxicity (ADCC), which are mediated through the Fc receptor binding, by maintaining a high degree of similarity to naturally occurring antibodies.

In WO 2007/024715 are provided dual variable domain immunoglobulins as engineered multivalent and multispecific binding proteins. A process for the preparation of biologically active antibody dimers is provided in U.S. Pat. No. 6,897,044. Multivalent FV antibody construct having at least four variable domains which are linked with each over via peptide linkers are provided in U.S. Pat. No. 7,129,330. Dimeric and multimeric antigen binding structures are provided in US 2005/0079170. Tri- or tetra-valent monospecific antigen-binding protein comprising three or four Fab fragments bound to each other covalently by a connecting structure, which protein is not a natural immunoglobulin are provided in U.S. Pat. No. 6,511,663. In WO 2006/020258 tetravalent bispecific antibodies are provided that can be efficiently expressed in prokaryotic and eukaryotic cells, and are useful in therapeutic and diagnostic methods. A method of separating or preferentially synthesizing dimers which are linked via at least one interchain disulfide linkage from dimers which are not linked via at least one interchain disulfide linkage from a mixture comprising the two types of polypeptide dimers is provided in US 2005/0163782. Bispecific tetravalent receptors are provided in U.S. Pat. No. 5,959,083. Engineered antibodies with three or more functional antigen binding sites are provided in WO 2001/077342.

Multispecific and multivalent antigen-binding polypeptides are provided in WO 97/001580. WO 92/004053 reports homoconjugates, typically prepared from monoclonal antibodies of the IgG class which bind to the same antigenic determinant are covalently linked by synthetic cross-linking.

WO 91/06305 whereby the oligomers, typically of the IgG class, are secreted having two or more immunoglobulin monomers associated together to form tetravalent or hexavalent IgG molecules. Sheep-derived antibodies and engineered antibody constructs are provided in U.S. Pat. No. 6,350,860, which can be used to treat diseases wherein interferon gamma activity is pathogenic. In US 2005/0100543 are provided targetable constructs that are multivalent carriers of bi-specific antibodies, i.e., each molecule of a targetable construct can serve as a carrier of two or more bi-specific antibodies. Genetically engineered bispecific tetravalent antibodies are provided in WO 95/009917. In WO 2007/109254 stabilized binding molecules that consist of or comprise a stabilized scFv are provided.

In some embodiments, the antibody or antigen-binding fragment thereof or the antibody scaffold is a multispecific antibody, e.g. a bispecific antibody. Multispecific antibodies are monoclonal antibodies that have binding specificities for at least two different sites.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making bispecific antibody fragments (see, e.g., Holliger, P. et al., Proc. Natl. Acad. Sci. USA 90 (1993) 6444-6448); and using single-chain Fv (scFv) dimers (see, e.g. Gruber, M et al., J. Immunol. 152 (1994) 5368-5374); and preparing trispecific antibodies as described, e.g., in Tutt, A. et al., J. Immunol. 147 (1991) 60-69).

In some embodiments, the antibody or antigen-binding fragment thereof or the antibody scaffold is an antibody scaffold.

### B. Engineered Disulfide Bonds

In some embodiments, the antibody or antigen-binding fragment thereof or the antibody scaffold comprises a variable domain and a constant domain, and the one or more engineered disulfide bonds are between a cysteine located in the variable domain and a cysteine located in the constant domain.

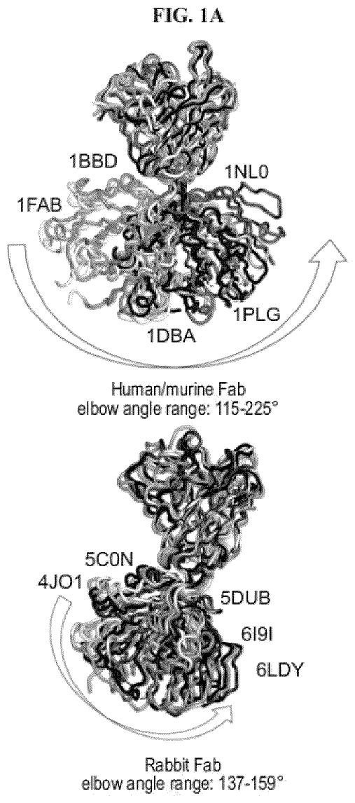

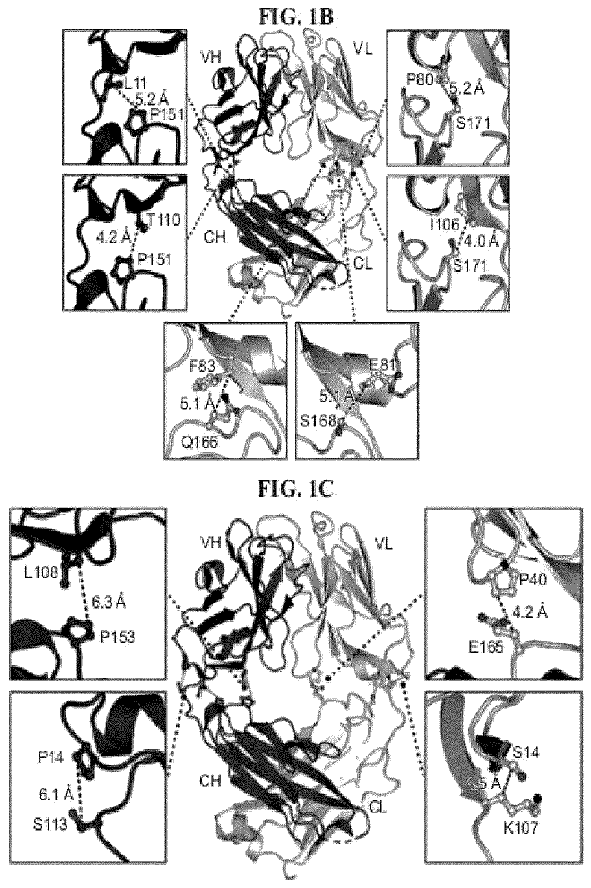

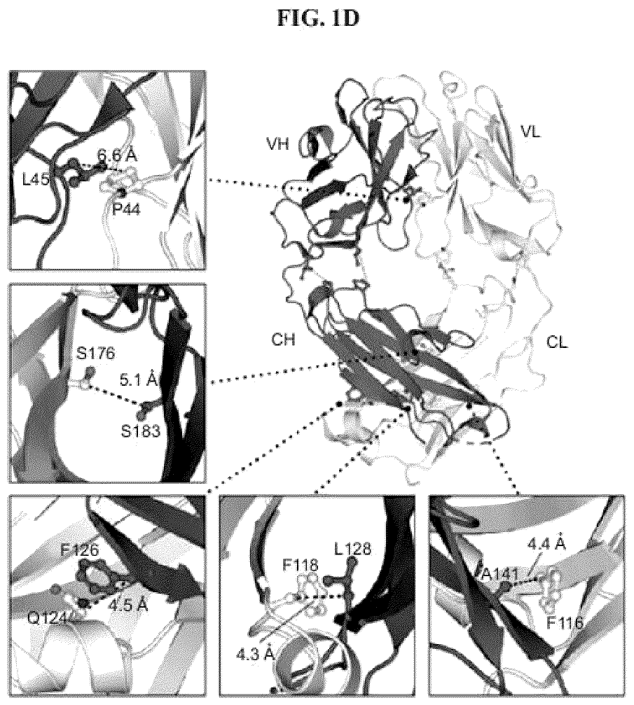

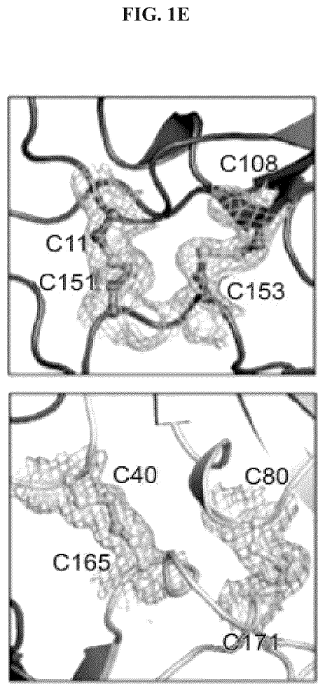

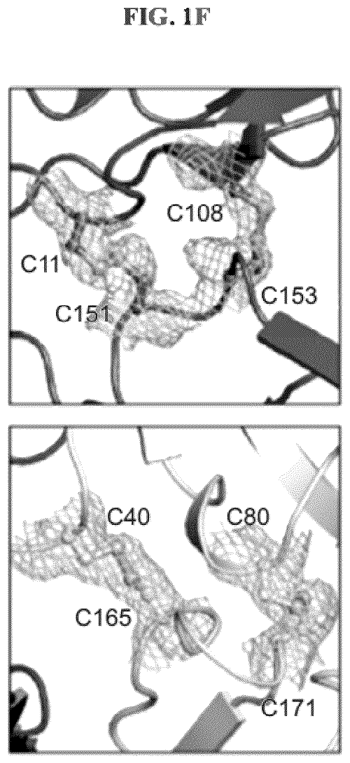

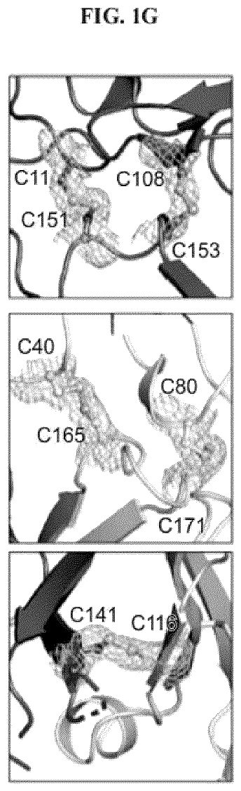

The elbow angle of a Fab antibody fragment is the angle between the two, not necessarily intersecting, pseudo-dyad axes relating the light (VL) and heavy (VH) chain variable domains, and the light (CL) and heavy (CH1) chain constant domains. Crystal structures of Fabs have revealed that there is a high degree of variability in the elbow angle between the variable and constant domains (115-225°) (Stanfield et al, J Mol Biol., 357:1566-1574, 2006; FIG. 1A). For example, lambda LCs are slightly longer, which leads to a wider range of possible elbow angles (Stanfield et al). On the other hand, analysis of structural alignments of existing crystal structures of Fabs from various species and frameworks revealed that rabbit Fabs are naturally less flexible than human or murine Fabs. (FIG. 1A, Table 1). In some embodiments, the one or more engineered disulfide bonds reduces the variability in the elbow angle between the variable domain and the constant domain of the antibody or fragment thereof.

In some embodiments, the antibody or antigen-binding fragment thereof or the antibody scaffold has one or more engineered disulfide bonds which restrict its flexibility. In some embodiments, the antibody or antigen-binding fragment thereof or the antibody scaffold has a less flexible elbow compared to an antibody or antigen-binding fragment thereof or the antibody scaffold that does not have the engineered disulfide bonds. In some embodiments, the engineered disulfide bond reduces the range of available conformations of the antibody or antigen-binding fragment thereof or the antibody scaffold. In some embodiments, the elbow angle range is 7 degrees or less. In some embodiments, the elbow angle range is 6 degrees or less. In some embodiments, the elbow angle range is 5 degrees or less. In some embodiments, the elbow angle range is 4 degrees or less. In some embodiments, the elbow angle range is 3 degrees or less. Herein, “elbow angle range” refers to the difference between the maximum and minimum elbow angle in the available conformations of the antibody or antigen-binding fragment thereof or the antibody scaffold.

In some embodiments, each of the one or more engineered disulfide bonds are between two amino acids that are positioned within a distance of between about 3.0 Å to about 7.0 Å of each other in one conformation of the antibody or antigen-binding fragment thereof or the antibody scaffold. In some embodiments, each of the one or more engineered disulfide bonds are between two amino acids that are positioned within a distance of between about 3.0 Å to about 4.0 Å of each other in one conformation of the antibody or antigen-binding fragment thereof or the antibody scaffold. In some embodiments, each of the one or more engineered disulfide bonds are between two amino acids that are positioned within a distance of between about 4.0 Å to about 5.0 Å of each other in one conformation of the antibody or antigen-binding fragment thereof or the antibody scaffold. In some embodiments, each of the one or more engineered disulfide bonds are between two amino acids that are positioned within a distance of between about 5.0 Å to about 6.0 Å of each other in one conformation of the antibody or antigen-binding fragment thereof or the antibody scaffold. In some embodiments, each of the one or more engineered disulfide bonds are between two amino acids that are positioned within a distance of between about 6.0 Å to about 7.0 Å of each other in one conformation of the antibody or antigen-binding fragment thereof or the antibody scaffold. In some embodiments, each of the one or more engineered disulfide bonds are between two amino acids that are positioned within 5.5 Å of each other in one conformation of the antibody or antigen-binding fragment thereof or the antibody scaffold. Distance between two amino acids can be determined using computational tools known in the art, such as PyMol. In some embodiments, the distance between two amino acids is measured by the distance between their respective C-beta atoms. In some embodiments, each of the one or more engineered disulfide bonds are between two amino acids whose C-beta atoms are positioned within 3.0 Å to about 7.0 Å of each other in one conformation of the antibody or antigen-binding fragment thereof or the antibody scaffold. Distance between two C-beta atoms can be determined using computational tools known in the art, such as PyMol.

In some embodiments, the engineered disulfide bond is between two amino acids that are positioned within a distance of between about 3.0 Å to about 7.0 Å of each other in one conformation and are positioned farther than a distance of between about 3.0 Å to about 7.0 Å of each other in another confirmation of the antibody or antigen-binding fragment thereof or the antibody scaffold. In some embodiments, a disulfide bond forms when the two amino acids come within a distance of between about 3.0 Å to about 7.0 Å of each other. In some embodiments, the engineered disulfide bond is between two amino acids that are positioned within a distance of between about 3.0 Å to about 4.0 Å of each other in one conformation and are positioned farther than a distance of between about 3.0 Å to about 4.0 Å of each other in another confirmation of the antibody or antigen-binding fragment thereof or the antibody scaffold. In some embodiments, a disulfide bond forms when the two amino acids come within a distance of between about 3.0 Å to about 4.0 Å of each other. In some embodiments, the engineered disulfide bond is between two amino acids that are positioned within a distance of between about 4.0 Å to about 5.0 Å of each other in one conformation and are positioned farther than a distance of between about 4.0 Å to about 5.0 Å of each other in another confirmation of the antibody or antigen-binding fragment thereof or the antibody scaffold. In some embodiments, a disulfide bond forms when the two amino acids come within a distance of between about 5.0 Å to about 6.0 Å of each other. In some embodiments, the engineered disulfide bond is between two amino acids that are positioned within a distance of between about 5.0 Å to about 6.0 Å of each other in one conformation and are positioned farther than a distance of between about 5.0 Å to about 6.0 Å of each other in another confirmation of the antibody or antigen-binding fragment thereof or the antibody scaffold. In some embodiments, a disulfide bond forms when the two amino acids come within a distance of between about 5.0 Å to about 6.0 Å of each other. In some embodiments, a disulfide bond forms when the two amino acids come within a distance of between about 6.0 Å to about 7.0 Å of each other. In some embodiments, the engineered disulfide bond is between two amino acids that are positioned within a distance of between about 6.0 Å to about 7.0 Å of each other in one conformation and are positioned farther than a distance of between about 6.0 Å to about 7.0 Å of each other in another confirmation of the antibody or antigen-binding fragment thereof or the antibody scaffold. In some embodiments, a disulfide bond forms when the two amino acids come within a distance of between about 6.0 Å to about 7.0 Å of each other. In some embodiments, the distance between two amino acids is measured by the distance between their respective C-beta atoms. In some embodiments, a disulfide bond forms when the C-beta atoms of two amino acids come within a distance of a distance of between about 3.0 Å to about 7.0 Å of each other in one conformation and are positioned farther than a distance of between about 3.0 Å to about 7.0 Å of each other in another conformation of the antibody or antigen-binding fragment thereof or the antibody scaffold.

In some embodiments, the engineered disulfide bond is between two amino acids that are positioned within about 5.5 Å of each other in one conformation and are positioned farther than about 5.5 Å of each other in another conformation of the antibody or antigen-binding fragment thereof or the antibody scaffold. In some embodiments, a disulfide bond forms when the two amino acids come within about 5.5 Å of each other. In some embodiments, the engineered disulfide bond is between two amino acids wherein their C-beta atoms are positioned within about 5.5 Å of each other in one conformation and are positioned farther than about 5.5 Å of each other in another conformation of the antibody or antigen-binding fragment thereof or the antibody scaffold. In some embodiments, a disulfide bond forms when the C-beta atoms of two amino acids come within about 5.5 Å of each other.

In some embodiments, identifying residue pairs to substitute with cysteine, thereby allowing disulfide bond formation, comprises analyzing crystal structures of antibodies or antigen-binding fragments thereof and identifying pairs of residues in close proximity (e.g., within 5.5 Å of each other in one conformation or in one structure of the antibody or antigen-binding fragment thereof or the antibody scaffold). In some embodiments, multiple crystal structures of the antibody may be used to identify amino acids that are suitable for forming disulfide bonds. In some embodiments, a pair of amino acids that is within about 5.5 Å of each other in one crystal structure is selected for substitution with cysteine. In some embodiments, a pair of amino acids that is within a distance of between about 3.0 Å to about 7.0 Å of each other in one crystal structure is selected for substitution with cysteine. In some embodiments, a pair of amino acids that is within a distance of between about 3.0 Å to about 4.0 Å, about 4.0 Å to about 5.0 Å, about 5.0 Å to about 6.0 Å or about 6.0 Å to about 7.0 Å of each other in one crystal structure is selected for substitution with cysteine. In some embodiments, the analysis of crystal structures comprises computational modeling.

In some embodiments, the distance described herein between two amino acids is the distance between the respective C-beta atom of the two amino acids.

In some embodiments, the antibody comprises two, three, four, five, six, seven, eight, nine, or ten or more engineered disulfide bonds. In some embodiments, the antibody comprises two engineered disulfide bonds. In some embodiments, the antibody comprises three engineered disulfide bonds. In some embodiments, the antibody comprises four engineered disulfide bonds. In some embodiments, the antibody comprises five engineered disulfide bonds. In some embodiments, the antibody comprises six engineered disulfide bonds. In some embodiments, the antibody comprises seven engineered disulfide bonds. In some embodiments, the antibody comprises eight engineered disulfide bonds. In some embodiments, the antibody comprises two or four engineered disulfide bonds. In some embodiments, the antibody comprises at least two engineered disulfide bonds. In some embodiments, the antibody comprises at least four engineered disulfide bonds. In some embodiments, the antibody comprises between two to four engineered disulfide bonds. In some embodiments, the antibody comprises between two to six engineered disulfide bonds. In some embodiments, the antibody comprises between two to eight engineered disulfide bonds. In some embodiments, the antibody comprises between four to six engineered disulfide bonds.

In some embodiments, the antibody comprises one or more disulfide bonds between the heavy chain constant domain and one or more disulfide bonds between the light chain constant domain and the light chain variable domain. In some embodiments, the antibody comprises one disulfide bond between the heavy chain constant domain and one disulfide bond between the light chain constant domain and the light chain variable domain. In some embodiments, the antibody comprises two disulfide bonds between the heavy chain constant domain and two disulfide bonds between the light chain constant domain and the light chain variable domain. In some embodiments, the antibody comprises one or more engineered disulfides between the light chain constant domain and the heavy chain constant domain.

Here, various positions for the engineered disulfide bonds are described, using Kabat numbering, with a semicolon separating a residue pair between which a disulfide bond is formed. “HC” in superscript after a position number indicates that the residue is in the heavy chain and “LC” in superscript after a position number indicates that the residue is in the light chain. In some embodiments, a residue pair is both mutated to cysteine at the positions indicated below. For example, 11^{HC}:151^{HC} means position 11 in the heavy chain is substituted with a cysteine, and position 151 in the heavy chain is substituted with a cysteine, thereby allowing the formation of a disulfide bond between the two positions. In some embodiments, a cysteine is inserted between positions x and y according to Kabat numbering, and this is denoted as [x]_[y]insC. For example, 108^{HC}:152^{HC}_153^{HC}insC means that position 108 in the heavy chain is substituted with a cysteine, and a cysteine is inserted between positions 152 and 153 in the heavy chain, thereby allowing the formation of a disulfide bond between the two cysteines. In some embodiments, the antibody or antigen-binding fragment thereof or the antibody scaffold comprises an insertion of a cysteine before or after the identified amino acid residue positions.

In some embodiments, the antibody or antigen-binding fragment thereof or the antibody scaffold comprises a kappa light chain or a lambda light chain. Kappa and lambda light chains have different CDR physicochemical and structural properties. Lambda light chains are slightly longer than kappa light chains, which leads to a wider range of possible elbow angles. Fabs with a lambda light chain are highly flexible. In some embodiments, an antibody or antigen-binding fragment thereof or the antibody scaffold comprising a lambda light chain comprises a disulfide bridge at a different position from an antibody or antigen-binding fragment thereof or the antibody scaffold comprising a kappa light chain.

In some embodiments, the antibody or antigen-binding fragment comprises an engineered disulfide bond such as 11^{HC}:151^{HC}, 108^{HC}:152^{HC}_153^{HC}insC, 40^{LC}:165^{LC}, 40^{LC}:166^{LC}, 80^{LC}:171^{LC}, 141^{HC}:116^{LC}, 126^{HC}:124^{LC}, 80^{LC}:170^{LC}_171^{LC}insC, 111^{HC}:151^{HC}, 106^{LC}:171^{LC}, 83^{LC}:166^{LC}, 81^{LC}:168^{LC}, 14^{HC}:113^{HC}, 14^{HC}:107^{HC}, 45^{HC}:44^{LC}, 183^{HC}:176^{LC} and 128^{HC}:118^{LC}. In some embodiments, the antibody or antigen-binding fragment thereof or the antibody scaffold is an antibody. In some embodiments, the antibody or antigen-binding fragment thereof or the antibody scaffold is an antigen-binding fragment of an antibody. In some embodiments, the antigen-binding fragment of an antibody is a Fab. In some embodiments, the antibody or antigen-binding fragment thereof or the antibody scaffold is an antibody scaffold.

In some embodiments, the antibody or antigen-binding fragment thereof or the antibody scaffold comprises a kappa light chain. In some embodiments, the antibody or antigen-binding fragment thereof or the antibody scaffold comprises an engineered disulfide bond such as 11^{HC}:151^{HC} and 80^{LC}:71^{LC}. In some embodiments, the antibody or antigen-binding fragment thereof or the antibody scaffold comprises an engineered disulfide bond such as 11^{HC}:151^{HC}, 108^{HC}:152^{HC}_153^{HC}insC, and 40^{LC}:165^{LC} and/or 80^{LC}:171^{LC}. In some embodiments, the antibody or antigen-binding fragment thereof or the antibody scaffold comprises an engineered disulfide bond such as 11^{HC}:151^{HC}, 108^{HC}:152^{HC}_153^{HC}insC 40^{LC}:165^{LC},80^{LC}:171^{LC} and 141^{HC}:116^{LC}. In some embodiments, the antibody or antigen-binding fragment thereof or the antibody scaffold comprises an engineered disulfide bond such as 11^{HC}:151^{HC}, 108^{HC}:152^{HC}_153^{HC}insC, 40^{LC}:165^{LC}, 80^{LC}:171^{LC},141^{HC}:116^{LC} and 126^{HC}:124^{LC}.

In some embodiments, the antibody or antigen-binding fragment thereof or the antibody scaffold comprises a lambda light chain. In some embodiments, the antibody or antigen-binding fragment thereof or the antibody scaffold comprises an engineered disulfide bond such as 11^{HC}:151^{HC} and 80^{LC}:170^{LC}_171^{LC}insC. In some embodiments, the antibody or antigen-binding fragment thereof or the antibody scaffold comprises an engineered disulfide bond such as 11^{HC}:151^{HC}, 108^{HC}:152^{HC}_153^{HC}insC, 40^{LC}, 166^{LC} and/or 80^{LC}:170^{LC}_171^{LC}insC. In some embodiments, the antibody or antigen-binding fragment thereof or the antibody scaffold comprises an engineered disulfide bond such as 11^{HC}:151^{HC}, 108^{HC}:152^{HC}_153^{HC}insC, 40^{LC}:166^{LC}, 80^{LC}:170^{LC}_171^{LC}insC, and 141^{HC}:116^{LC}. In some embodiments, the antibody or antigen-binding fragment thereof or the antibody scaffold comprises an engineered disulfide bond such as 11^{HC}:151^{HC}, 108^{HC}:152^{HC}_153^{HC}insC, 40^{LC}:166^{LC}, 80^{LC}:170^{LC}_171^{LC} insC, 141^{HC}: 16^{LC}, and 126^{HC}:124^{LC}. In some embodiments, the antibody or antigen-binding fragment thereof or the antibody scaffold is an antibody. In some embodiments, the antibody or antigen-binding fragment thereof or the antibody scaffold is an antigen-binding fragment of an antibody. In some embodiments, the antigen-binding fragment of an antibody is a Fab. In some embodiments, the antibody or antigen-binding fragment thereof or the antibody scaffold is an antibody scaffold.

Disulfide bonds are formed by the oxidation of two cysteine residues to result in a cystine. In some embodiments, the cystine or disulfide bond can form during cellular expression and folding of the antibody or antigen-binding fragment thereof or the antibody scaffold. In some embodiments, the antibody or antigen-binding fragment thereof or the antibody scaffold is incubated with an oxidizing agent known in the art to promote formation of disulfide bonds.

In some embodiments, the methods described herein are performed in vitro.

In some embodiments, the antibody or antigen-binding fragment thereof or the antibody scaffold is an antibody comprising a VL region and a VH region, wherein the VL has an amino acid sequence according to SEQ ID NO: 1 and the VH has an amino acid sequence according to SEQ ID NO: 2. In some embodiments, the antibody or antigen-binding fragment thereof or the antibody scaffold is an antibody comprising a VL region and a VH region, wherein the VL has an amino acid sequence according to SEQ ID NO: 3 and the VH has an amino acid sequence according to SEQ ID NO: 4. In some embodiments, the antibody or antigen-binding fragment thereof or the antibody scaffold is an antibody comprising a VL region and a VH region, wherein the VL has an amino acid sequence according to SEQ ID NO: 5 and the VH has an amino acid sequence according to SEQ ID NO: 6. In some embodiments, the antibody or antigen-binding fragment thereof or the antibody scaffold is an antibody comprising a VL region and a VH region, wherein the VL has an amino acid sequence according to SEQ ID NO: 7 and the VH has an amino acid sequence according to SEQ ID NO: 8. In some embodiments, the antibody or antigen-binding fragment thereof or the antibody scaffold is an antibody comprising a VL region and a VH region, wherein the VL has an amino acid sequence according to SEQ ID NO: 9 and the VH has an amino acid sequence according to SEQ ID NO: 10. In some embodiments, the antibody or antigen-binding fragment thereof or the antibody scaffold is an antibody comprising a VL region and a VH region, wherein the VL has an amino acid sequence according to SEQ ID NO: 11 and the VH has an amino acid sequence according to SEQ ID NO: 12. In some embodiments, the antibody or antigen-binding fragment thereof or the antibody scaffold is an antibody comprising a VL region and a VH region, wherein the VL has an amino acid sequence according to SEQ ID NO: 13 and the VH has an amino acid sequence according to SEQ ID NO: 14. In some embodiments, the antibody or antigen-binding fragment thereof or the antibody scaffold is an antibody comprising a VL region and a VH region, wherein the VL has an amino acid sequence according to SEQ ID NO: 15 and the VH has an amino acid sequence according to SEQ ID NO: 16. In some embodiments, the antibody or antigen-binding fragment thereof or the antibody scaffold is an antibody comprising a VL region and a VH region, wherein the VL has an amino acid sequence according to SEQ ID NO: 17 and the VH has an amino acid sequence according to SEQ ID NO: 18. In some embodiments, the antibody or antigen-binding fragment thereof or the antibody scaffold is an antibody comprising a VL region and a VH region, wherein the VL has an amino acid sequence according to SEQ ID NO: 19 and the VH has an amino acid sequence according to SEQ ID NO: 20. In some embodiments, the antibody or antigen-binding fragment thereof or the antibody scaffold is an antibody comprising a VL region and a VH region, wherein the VL has an amino acid sequence according to SEQ ID NO: 21 and the VH has an amino acid sequence according to SEQ ID NO: 22. In some embodiments, the antibody or antigen-binding fragment thereof or the antibody scaffold is an antibody comprising a VL region and a VH region, wherein the VL has an amino acid sequence according to SEQ ID NO: 23 and the VH has an amino acid sequence according to SEQ ID NO: 24. In some embodiments, the antibody or antigen-binding fragment thereof or the antibody scaffold is an antibody comprising a VL region and a VH region, wherein the VL has an amino acid sequence according to SEQ ID NO: 25 and the VH has an amino acid sequence according to SEQ ID NO: 26. In some embodiments, the antibody or antigen-binding fragment thereof or the antibody scaffold is an antibody. In some embodiments, the antibody or antigen-binding fragment thereof or the antibody scaffold is an antigen-binding antibody fragment of an antibody. In some embodiments, the antigen-binding antibody fragment is a Fab. In some embodiments, the antibody or antigen-binding fragment thereof or the antibody scaffold is an antibody scaffold.

Table 6 provides an overview of the exemplary antibodies used to illustrate certain embodiments of the technology disclosed herein.

### C. Molecule

In some embodiments, the antibody or antigen-binding fragment thereof or the antibody scaffold binds to a molecule. In some embodiments, the molecule is a small molecule, peptide, protein, fusion protein, nucleic acid, or a lipid. As used herein, a “small molecule” is an organic molecule characterized by a mass of less than 900 Daltons. In some embodiments, a small molecule is a compound with low molecular weight, e.g., less than 1 kDa. In some embodiments, the molecule is a therapeutic small molecule drug.

In some embodiments, the molecule is a fusion protein. Non-limiting examples of fusion proteins include Protein A-Protein G (ProA-ProG) fusion protein, fusion of a protein/peptide drug with an antibody Fc fragment, BCR-ABL fusion protein, tandem fusion proteins, proteins with domain insertions, and proteins joined by protein linkers.

In some embodiments, the molecule is a nucleic acid and/or protein. The molecule may include any biomolecule or chemical compound, including a macromolecule such as a protein or peptide, a lipid or a nucleic acid molecule, or a small molecule, including organic or inorganic molecules.

In some embodiments, the molecule is a nucleic acid molecule, such as DNA (e.g. genomic DNA, mitochondrial DNA, plastid DNA, viral DNA, etc.) and RNA (e.g., mRNA, microRNA, rRNA, snRNA, viral RNA, etc.), and synthetic and/or modified nucleic acid molecules, (e.g., including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. In some embodiments, the molecule is a single molecule or a complex that contains two or more molecular subunits, e.g., including but not limited to protein-DNA complexes, which may or may not be covalently bound to one another, and which may be the same or different. In some embodiments, the molecule is a protein complex or protein interaction. Such a complex or interaction may thus be a homo- or hetero-multimer. In some embodiments, the molecule is a complex between proteins or peptides and nucleic acid molecules such as DNA or RNA, e.g., interactions between proteins and nucleic acids, e.g., regulatory factors, such as transcription factors, and DNA or RNA.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, the molecule is a cytokine, a drug, or an enzyme substrate. In some embodiments, the cytokine is an interleukin. In some embodiments, the interleukin is selected from the group consisting of IL-1α, IL-1β, IL-18, IL-33, IL-36u, IL-36p, IL-36y, IL-1, IL-2, IL-3, IL-4, IL-5, IL-6, IL-7, IL-8, IL-9, IL-10, IL-11, IL-12, IL-13, IL-14, and IL-15.

In some embodiments, the molecule is a therapeutic agent. The therapeutic agent can be any compound, moiety or group which has a cytotoxic or cytostatic effect. Drug moieties include: (i) chemotherapeutic agents, which may function as microtubule inhibitors, mitosis inhibitors, topoisomerase inhibitors, or DNA intercalators; (ii) protein toxins, which may function enzymatically; and (iii) radioisotopes. Exemplary therapeutic agents include, but are not limited to, a maytansinoid, an auristatin, a dolastatin, a trichothecene, CC1065, a calicheamicin and other enediyne antibiotics, a taxane, an anthracycline, and stereoisomers, isosters, analogs or derivatives thereof. Protein toxins include diphtheria-A chain, non-binding active fragments of diphtheria toxin, exotoxin A chain (from Pseudomonas aeruginosa), ricin A chain (Vitetta et al (1987) Science, 238:1098), abrin A chain, modeccin A chain, alpha-sarcin, Aleurites fordii proteins, dianthin proteins, Phytolaca americana proteins (PAPI, PAPII, and PAP-5), Momordica charantia inhibitor, curcin, crotin, Sapaonaria officinalis inhibitor, gelonin, mitogellin, restrictocin, phenomycin, enomycin, and the tricothecenes (WO 93/21232).

In some embodiments, the antibody or antigen-binding fragment thereof or the antibody scaffold is bound covalently to the molecule. In some embodiments, the antibody or antigen-binding fragment thereof or the antibody scaffold is bound non-covalently to the molecule.

In some embodiments, the molecule is less than 50 kDa. In some embodiments, the molecule is less than 40 kDa. In some embodiments, the molecule is less than 30 kDa. In some embodiments, the molecule is less than 20 kDa. In some embodiments, the molecule is less than 10 kDa. In some embodiments, the molecule is greater than 50 kDa. In some embodiments, the molecule is greater than 100 kDa. In some embodiments, the molecule is greater than 150 kDa.

In some embodiments, the molecule is about 10-50 kDa. In some embodiments, the molecule is about 1-10 kDa. In some embodiments, the molecule is about 1-20 kDa. In some embodiments, the molecule is about 1-30 kDa. In some embodiments, the molecule is about 1-40 kDa. In some embodiments, the molecule is about 1-50 kDa. In some embodiments, the molecule is about 50-100 kDa. In some embodiments, the molecule is about 100-150 kDa. In some embodiments, the molecule is about 150-200 kDa.

### D. Expression and Purification of Antibodies

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody as provided herein is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody as provided herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P. et al., Annals N.Y. Acad. Sci. 383 (1982) 44-68; MRC 5 cells; and FS4 cells. Other useful mammalian host cell lines include Chinese hamster ovary (CHO) cells, including DHFR⁻ CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

### E. Complexes

In some embodiments, the antibody or antigen-binding fragment thereof or the antibody scaffold forms a complex with a molecule. In some embodiments, the complex comprising the molecule and the antibody or fragment thereof is a non-covalent complex. In some embodiments, the complex comprising the molecule and the antibody or fragment thereof is a covalent complex.

In some embodiments, the antibody or antigen-binding fragment thereof or the antibody scaffold and the molecule are tightly bound in the complex. This can be accomplished by, e.g., crosslinking the antibody or antigen-binding fragment thereof or the antibody scaffold to the molecule and/or the antibody or antigen-binding fragment thereof or the antibody scaffold having sufficiently high (i.e., micromolar/nanomolar/picomolar/femtomolar) affinity for the molecule.

In some embodiments, the antibody or antigen-binding fragment thereof or the antibody scaffold binds the molecule with high (i.e., micromolar/nanomolar/picomolar/femtomolar) affinity. The affinity can be measured by the Kd value of the antibody or antigen-binding fragment thereof or the antibody scaffold. In some embodiments, the antibody or antigen-binding fragment thereof or the antibody scaffold comprising one or more engineered disulfide bonds binds to the molecule with affinity similar to that of the wildtype antibody. In some embodiments, the Kd of the antibody or antigen-binding fragment thereof or the antibody scaffold is better (i.e., tighter) than 100 nM. In some embodiments, the Kd of the antibody or antigen-binding fragment thereof or the antibody scaffold is better than 10 nM. In some embodiments, the Kd of the antibody or antigen-binding fragment thereof or the antibody scaffold is better than 1 nM. In some embodiments, the Kd of the antibody or antigen-binding fragment thereof or the antibody scaffold is between 100 nM and 1 nM. In some embodiments, the Kd of the antibody is between 100 nM and 10 nM. In some embodiments, the Kd of the antibody or antigen-binding fragment is between 10 nM and 1 nM.

In some embodiments, the antibody or antigen-binding fragment thereof or the antibody scaffold and the molecule are crosslinked, e.g., with a crosslinking agent, e.g., with a chemical crosslinking agent. In some embodiments, the Kd of the antibody or antigen-binding fragment thereof or the antibody scaffold is worse (i.e., weaker affinity) than 10 μM. In some embodiments, the Kd of the antibody or antigen-binding fragment thereof or the antibody scaffold is better (i.e., tighter affinity) than 10 μM. In some embodiments, the Kd of the antibody or antigen-binding fragment thereof or the antibody scaffold is better than 1 μM. In some embodiments, the Kd of the antibody or antigen-binding fragment thereof or the antibody scaffold is better than 0.5 μM. In some embodiments, the Kd of the antibody or antigen-binding fragment thereof or the antibody scaffold is better than 0.3 μM. In some embodiments, the Kd of the antibody or antigen-binding fragment thereof or the antibody scaffold is better than 0.1 μM. In some embodiments, the Kd of the antibody or antigen-binding fragment thereof or the antibody scaffold is between 0.5 μM and 0.1 μM. In some embodiments, the Kd of the antibody or antigen-binding fragment thereof or the antibody scaffold is between 0.5 μM and 0.3 μM. In some embodiments, the Kd of the antibody or antigen-binding fragment thereof or the antibody scaffold is between 0.3 μM and 0.1 μM. In some embodiments, the Kd of the antibody or antigen-binding fragment thereof or the antibody scaffold is between 1 μM and 0.1 μM. In some embodiments, the Kd of the antibody or antigen-binding fragment thereof or the antibody scaffold is between 1 μM and 10 μM.

The molecule can be chemically crosslinked to the antibody or antigen-binding fragment thereof or the antibody scaffold through methods known in the art, e.g., as described in Stark, H. et al. (2010). GraFix: stabilization of fragile macromolecular complexes for single particle cryo-EM. Methods in Enzymology (Vol. 481, pp. 109-126). Academic Press. In some embodiments, the antibody or antigen-binding fragment thereof or the antibody scaffold and the molecule undergo a weak, intramolecular chemical cross-linking while being purified by density gradient ultracentrifugation. In some embodiments, the molecule is chemically cross-linked to the antibody or antigen-binding fragment thereof or the antibody scaffold. Common crosslinkers are known in the art. Crosslinkers include, but are not limited to, the imidoester crosslinker dimethyl suberimidate, the N-Hydroxysuccinimide-ester crosslinker BS3 and formaldehyde.

In some embodiments, the molecule is a small molecule, peptide, protein, nucleic acid, or a lipid. In some embodiments, the molecule is a cytokine, a drug, or an enzyme substrate. In some embodiments, a small molecule is a compound with low molecular weight, e.g., less than 1 kDa. In some embodiments, a small molecule is a compound with low molecular weight of 500 Da to 1 kDa. In some embodiments, a small molecule is a compound with low molecular weight of 10 Da to 500 Da.

In some embodiments, the complex comprising the molecule and the antibody or fragment thereof is at least 40 kDa. In some embodiments, the complex comprising the molecule and the antibody or fragment thereof is at least 50 kDa. In some embodiments, the complex comprising the molecule and the antibody or fragment thereof is at least 60 kDa. In some embodiments, the complex comprising the molecule and the antibody or fragment thereof is at least 70 kDa. In some embodiments, the complex comprising the molecule and the antibody or fragment thereof is at least 80 kDa. In some embodiments, the complex comprising the molecule and the antibody or fragment thereof is at least 90 kDa. In some embodiments, the complex comprising the molecule and the antibody or fragment thereof is at least 100 kDa. In some embodiments, the complex comprising the molecule and the antibody or fragment thereof is between about 40 kDa to about 60 kDa. In some embodiments, the complex comprising the molecule and the antibody or fragment thereof is between about 40 kDa to about 80 kDa. In some embodiments, the complex comprising the molecule and the antibody or fragment thereof is between about 40 kDa to about 100 kDa. In some embodiments, the complex comprising the molecule and the antibody or fragment thereof is between about 50 kDa to about 100 kDa. In some embodiments, the complex comprising the molecule and the antibody or fragment thereof is between about 60 kDa to about 100 kDa. In some embodiments, the complex comprising the molecule and the antibody or fragment thereof is between about 70 kDa to about 100 kDa. In some embodiments, the complex comprising the molecule and the antibody or fragment thereof is between about 80 kDa to about 100 kDa. In some embodiments, the complex comprising the molecule and the antibody or fragment thereof is between about 90 kDa to about 100 kDa. In some embodiments, the complex comprising the molecule and the antibody or fragment thereof is between about 40 kDa to about 1000 kDa. In some embodiments, the complex comprising the molecule and the antibody or fragment thereof is between about 100 kDa to about 1000 kDa.

In some embodiments, the antibody or antigen-binding fragment thereof or the antibody scaffold is an antibody scaffold.

### III. USES FOR STRUCTURAL DETERMINATION

In some aspects, provided herein are methods of determining a high-resolution structure of a molecule comprising determining a high-resolution structure of a complex comprising the molecule and an antibody or antigen-binding fragment thereof or the antibody scaffold that binds to the molecule, wherein the antibody or antigen-binding fragment comprises one or more engineered disulfide bonds that increase conformational rigidity of the antibody or antigen-binding fragment thereof or the antibody scaffold, and thereby determining the high-resolution structure of the molecule.

### A. Recombinant Protein Expression and Purification

In some embodiments, the method further comprises generating an antibody that binds to the molecule, prior to determining the high-resolution structure. In some embodiments, the method comprises generating the antibody or antigen-binding fragment thereof or the antibody scaffold through, e.g., molecular cloning or a synthetic biology method known in the art, e.g., gene synthesis. In some embodiments, the antibody or antigen-binding fragment can be generated by immunizing a mouse, rabbit, or other appropriate mammal known in the art. In some embodiments, the antibody or antigen-binding fragment can be generated by phage display. In some embodiments, the antibody or antigen-binding fragment thereof or the antibody scaffold can be generated using de novo immunization methods using inter alia either the antigen protein or nucleic acid or fragments thereof, or by phage display, or by yeast display or using computational methods. In some embodiments, the method comprises further expressing constructs of the antibody or antigen-binding fragment thereof or the antibody scaffold in Chinese Hamster Ovary cells or other mammalian cell lines for expression. In some embodiments, the antibody or antigen-binding fragment thereof or the antibody scaffold is incubated with an oxidizing agent known in the art to promote formation of disulfide bonds. In some embodiments, the method further comprises purifying the antibody or antigen-binding fragment thereof or the antibody scaffold by one or more forms of chromatography (e.g., nickel affinity chromatography followed by size exclusion chromatography). In some embodiments, the antibody or antigen-binding fragment comprises one or more engineered disulfide bonds, and the method further comprises confirming the presence of the one or more disulfide bonds by intact protein LC/MS or disulfide bond mapping using mass spectrometry.

### B. Cryogenic Electron Microscopy

The methods described herein are methods useful for the determination of a high-resolution cryogenic electron microscopy (cryo-EM) structure. In some embodiments, determining the high-resolution structure comprises performing cryo-EM on the complex comprising the molecule and the antibody or antigen-binding fragment thereof or the antibody scaffold.

In some embodiments, the method further comprises incubating the antibody or antigen-binding fragment thereof or the antibody scaffold with the molecule to form a complex prior to determining the high-resolution structure. In some embodiments, the antibody or antigen-binding thereof and the molecule are incubated on ice. In some embodiments, the antibody or fragment thereof and the molecule are incubated at about 4 degrees Celsius. In some embodiments, the antibody or antigen-binding fragment thereof or the antibody scaffold and the molecule are incubated at room temperature. In some embodiments, the antibody or antigen-binding fragment thereof or the antibody scaffold and the molecule are incubated for about 30 minutes. In some embodiments, the antibody or antigen-binding fragment thereof or the antibody scaffold and the molecule are incubated at room temperature for about 30 minutes. In some embodiments, the antibody or antigen-binding fragment thereof or the antibody scaffold and the molecule are incubated at about 4 degrees Celsius for about 30 minutes. In some embodiments, the antibody or antigen-binding fragment thereof or the antibody scaffold and the molecule are incubated on ice for about 30 minutes. In some embodiments, the antibody or antigen-binding fragment thereof or the antibody scaffold and the molecule are incubated at about 0 degrees Celsius to about 4 degrees Celsius for about 30 minutes.

In some embodiments, the method further comprises purifying the complex after the incubation. In some embodiments, the purification comprises separating the complex by size exclusion chromatography. In some embodiments, the purification comprises any one or more of the group consisting of: hydrophobic interaction column chromatography, size exclusion chromatography, ion exchange column chromatography, and affinity chromatography.

In some embodiments, the method further comprises subjecting the peak fraction of size exclusion chromatography to a mild crosslinking agent (e.g., Bissulfosuccinimidyl suberate). In some embodiments, the method further comprises crosslinking the complex using the gradient fixation method. See, Stark, H. (2010) GraFix: stabilization of fragile macromolecular complexes for single particle cryo-EM. In Methods in enzymology (Vol. 481, pp. 109-126). Academic Press. During GraFix, macromolecules undergo a weak, intramolecular chemical cross-linking while being purified by density gradient ultracentrifugation. GraFix-stabilized particles can be used directly for negative-stain cryo-EM or, after a brief buffer-exchange step, for unstained cryo-EM.

In some embodiments, prior to applying the complex to the grid, the grid is treated overnight with monothiolalkane(C11)PEG6-OH (11-mercaptoundecyl) hexaethyleneglycol. In some embodiments, the complex is applied to the grid at about 4 °C and about 100% humidity. In some embodiments, the method further comprises plunge-freezing the grid comprising the complex in liquid ethane cooled by liquid nitrogen.

In some embodiments, the method further comprises, prior to determining the high-resolution structure, producing the antibody comprising one or more engineered disulfide bonds that increase conformational rigidity of the antibody by (i) analyzing structural data from the antibody or antigen-binding fragment thereof or the antibody scaffold, or from an antibody obtained from the same species as the antibody or antigen-binding fragment thereof or the antibody scaffold; (ii) identifying amino acid residue positions in the constant domain and the variable domain that are sufficiently close to form a disulfide bond; and (iii) introducing a cysteine at the identified amino acid residue positions, or inserting a cysteine before or after the identified amino acid residue positions. In some embodiments, structural data is experimentally determined. In some embodiments, structural data is determined by modeling using in silico methods.

In some embodiments, the method further comprises processing cryo-EM images. In some embodiments, the processing comprises motion correction, CTF estimation, and particle picking. In some embodiments, the processing comprises rejecting images based on their CTF fit resolutions.

In some embodiments, the method further comprises performing refinement of an initial structure. In some embodiments, the method further comprises performing iterative rounds of multi-class ab initio reconstruction and heterogeneous refinement. In some embodiments, the method further comprises performing non-uniform refinement. In some embodiments, the method further comprises performing local refinement.

In some embodiments, the method of determining the high-resolution structure of the molecule comprises subjecting the refined cryo-EM 3D reconstruction to masking around the complex comprising the variable domain of the antibody or antigen-binding fragment thereof or the antibody scaffold and the molecule. In some embodiments, the method further comprises selecting and re-extracting the classes where the molecule was best resolved. In some embodiments, the method further comprises local refinement with a mask around the antibody or antigen-fragment thereof to improve alignments of the Fabs prior to particle subtraction to remove the Fabs. In some embodiments, the method further comprises particle subtraction to remove the Fabs, thereby determining the high-resolution structure of the molecule. In some embodiments, the method further comprises local refinement of the signal-subtracted particles with a mask around the molecule.

In some embodiments, the method further comprises performing validation of a structure to determine the high-resolution structure. In some embodiments, validation comprises determining and analyzing the molprobity score, clashscore, and/or percentage of poor rotamers.

In some embodiments, the overall resolution of the high-resolution structure of the complex is determined to be better than 4 Å, better than 3.5 Å, better than 3 Å or better than 2.8 Å. In some embodiments, the resolution of the high-resolution structure of the complex is determined to be better than 4 Å. In some embodiments, the resolution of the high-resolution structure of the complex is determined to be better than 3.5 Å. In some embodiments, the resolution of the high-resolution structure of the complex is determined to be better than 3 Å. In some embodiments, the resolution of the high-resolution structure of the complex is determined to be better than 2.8 Å.

In some embodiments, the overall resolution of the high-resolution structure of the molecule is determined to be better than 4 Å, better than 3.5 Å, better than 3 Å or better than 2.8 Å. In some embodiments, the resolution of the high-resolution structure of the molecule is determined to be better than 4 Å. In some embodiments, the resolution of the high-resolution structure of the molecule is determined to be better than 3.5 Å. In some embodiments, the resolution of the high-resolution structure of the molecule is determined to be better than 3 Å. In some embodiments, the resolution of the high-resolution structure of the molecule is determined to be better than 2.8 Å.

In some embodiments, the methods described herein are performed in vitro.

In some embodiments, described herein is a method of increasing the rigidity of an antibody or antigen-binding fragment thereof or of an antibody scaffold comprising a constant domain and a variable domain, the method comprising introducing a cysteine residue in the constant domain and introducing a cysteine residue in the variable domain, wherein the cysteine residue in the constant domain and the cysteine residue in the variable domain are sufficiently close in a confirmation to form an engineered disulfide bond, and allowing or inducing formation of the engineered disulfide bond, thereby increasing the rigidity of the antibody or antigen binding fragment thereof or of the antibody scaffold.

In some embodiments, the antibody or antigen-binding fragment thereof or the antibody scaffold is an antibody. In some embodiments, the antibody or antigen-binding fragment thereof or the antibody scaffold is an antigen-binding fragment of an antibody. In some embodiments, the antigen-binding fragment of an antibody is a Fab. In some embodiments, the antibody or antigen-binding fragment thereof or the antibody scaffold is an antibody scaffold.

The disclosure of all references cited herein is herewith incorporated by reference.

The following examples, figures and sequences are provided to aid the understanding of the present invention, the true scope of which is set forth in the appended claims. It is understood that modifications can be made in the procedures set forth without departing from the spirit of the invention.

### EXAMPLES

### Example 1: Rigid Fabs Enable High-Resolution Structure Determination of Small Proteins by Cryo-EM

This Example demonstrates a method wherein Rigid Fabs make cryo-EM accessible for all small proteins, enabling their structure determination to a high resolution (<3 Å).

### Design and Characterization of Rigid Fabs

Crystal structures of Fabs have revealed that there is a high degree of variability in the elbow angle between the variable and constant domains (115-225°) (FIG. 1A) of antibodies. This flexibility frequently causes the constant domain of the Fab to be poorly resolved in cryo-EM maps, and masks are often applied during refinement to exclude the Fab constant domain in order to achieve higher resolution at the Fab-antigen interface for large antigen-Fab complexes. Thus, only the ~25 kDa variable domain of the Fab is fully utilized in these cases to aid in image alignment. If the Fab were conformationally rigid with a distinctive shape, however, including the constant domain would (a) increase the overall ordered size of the particle by ~50 kDa, (b) alleviate the need for a mask to discount the constant domain, and (c) improve image alignment, leading to improved resolution of the constant domain and of the antigen-Fab complex overall. For a truly, conformationally rigid Fab, the local resolution throughout the Fab would be expected to be relatively uniform.

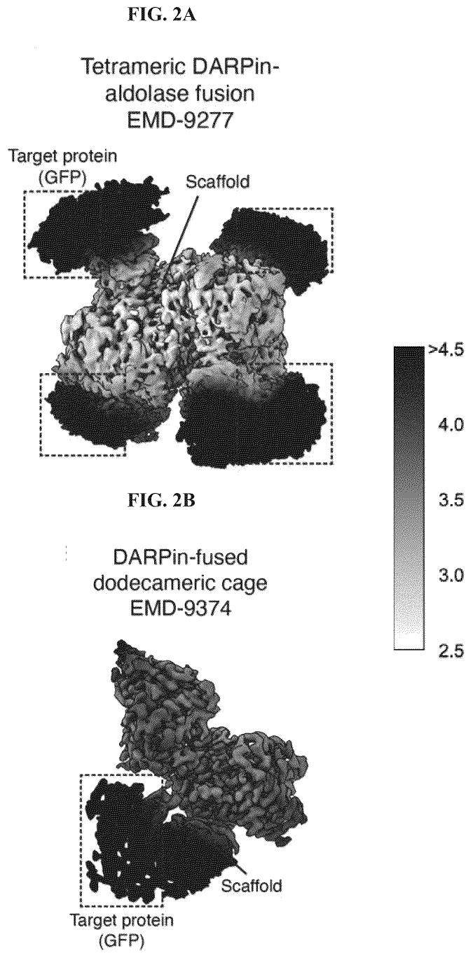

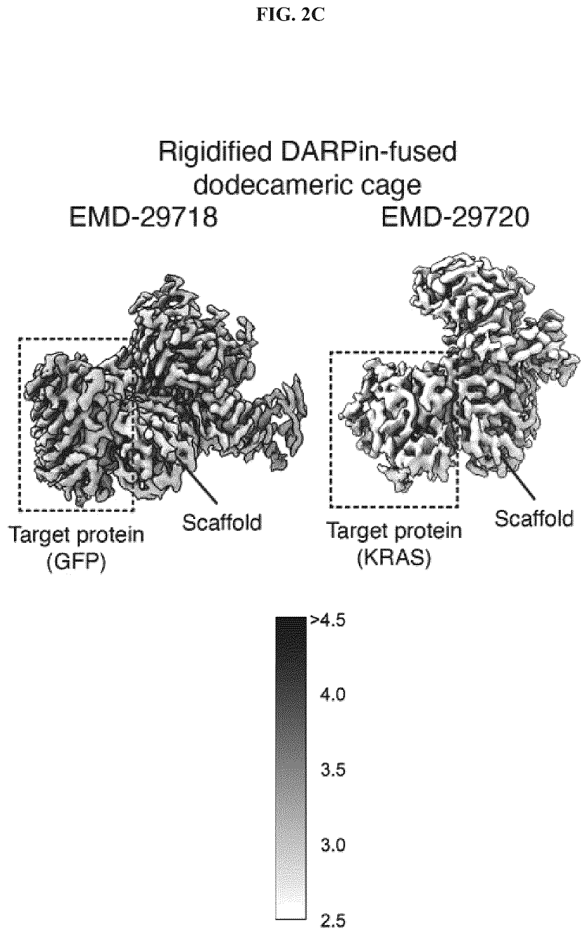

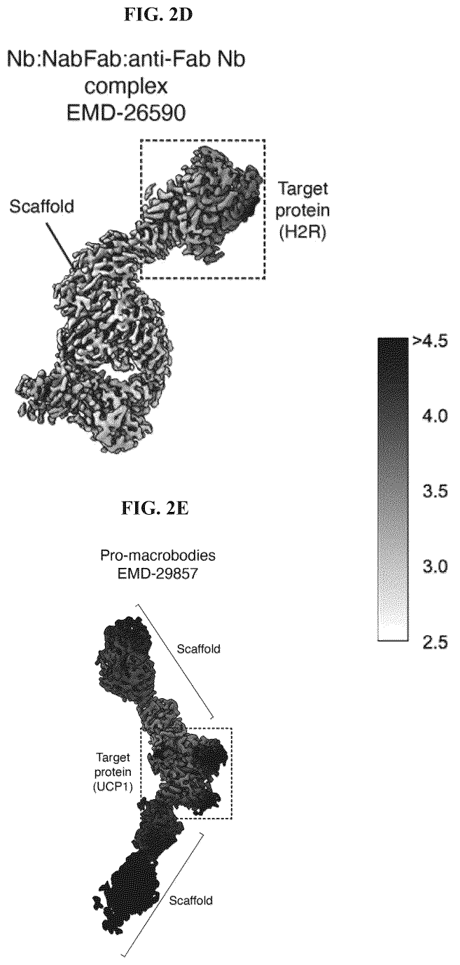

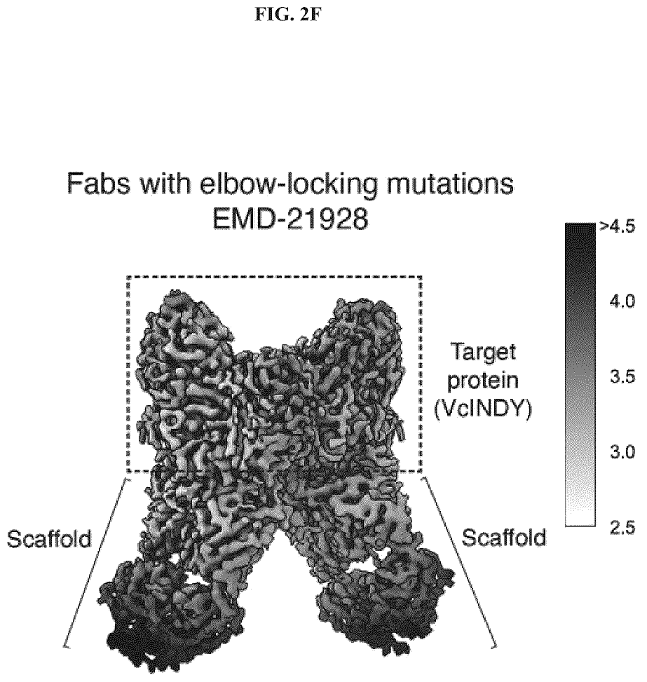

Previous attempts to generate conformationally rigid Fabs utilized phage display to shorten and mutate the heavy chain (HC) elbow of the Herceptin Fab framework, and clones were selected based on their thermal stability (Bailey et al, J Mol Biol, 430:337-347, 2018). However, use of Fabs with these modifications in cryo-EM studies revealed that they are still quite flexible (FIG. 2F). See, Zhang et al, Elife, 7:e33572, 2018; Lopez-Redondo, J Gen Physiol, 153:e202112873, 2021; Brawley et al, Nat Chem Biol, 18:706-712, 2022; Kintzer et al, PNAS, 115:E9095-E9104, 2018; and Sauer et al, Elife, 9:e61350, 2020.

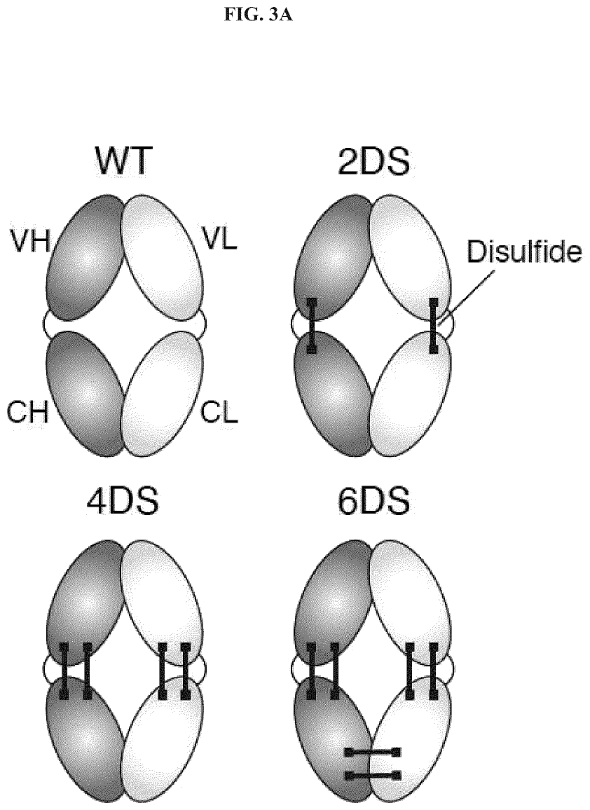

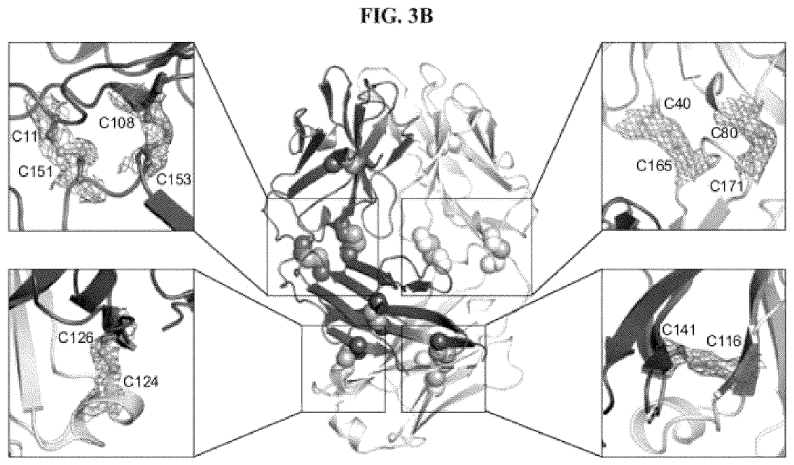

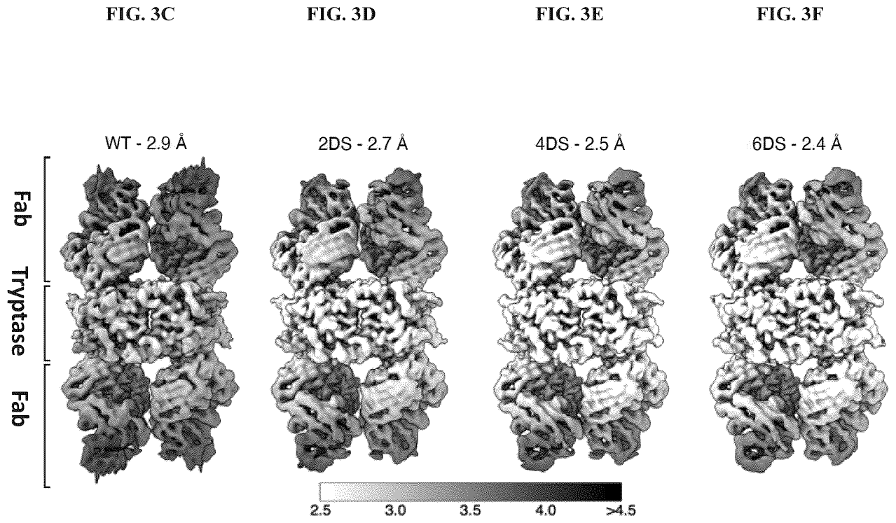

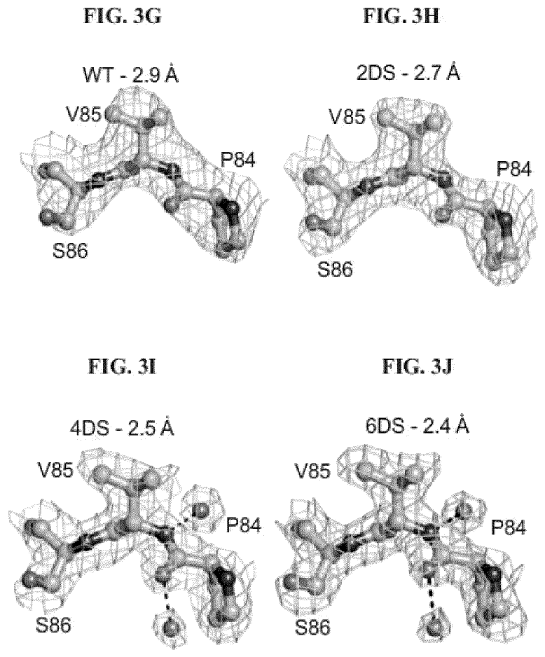

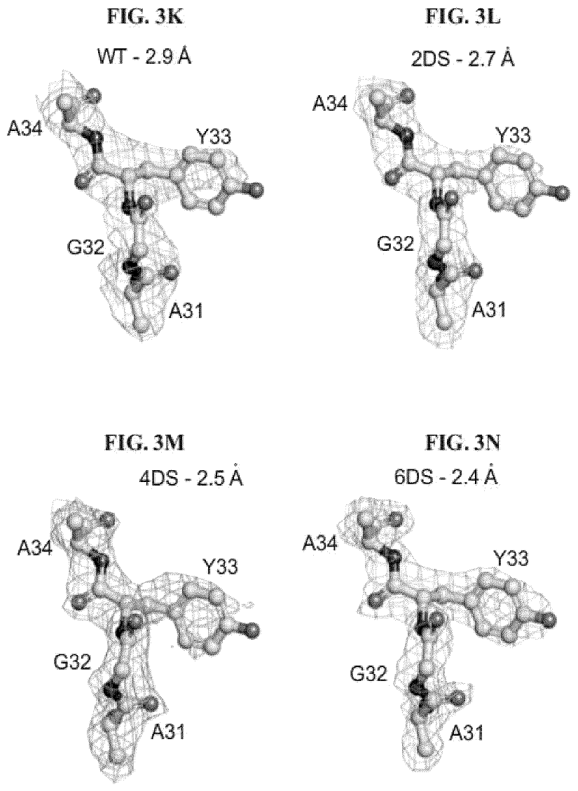

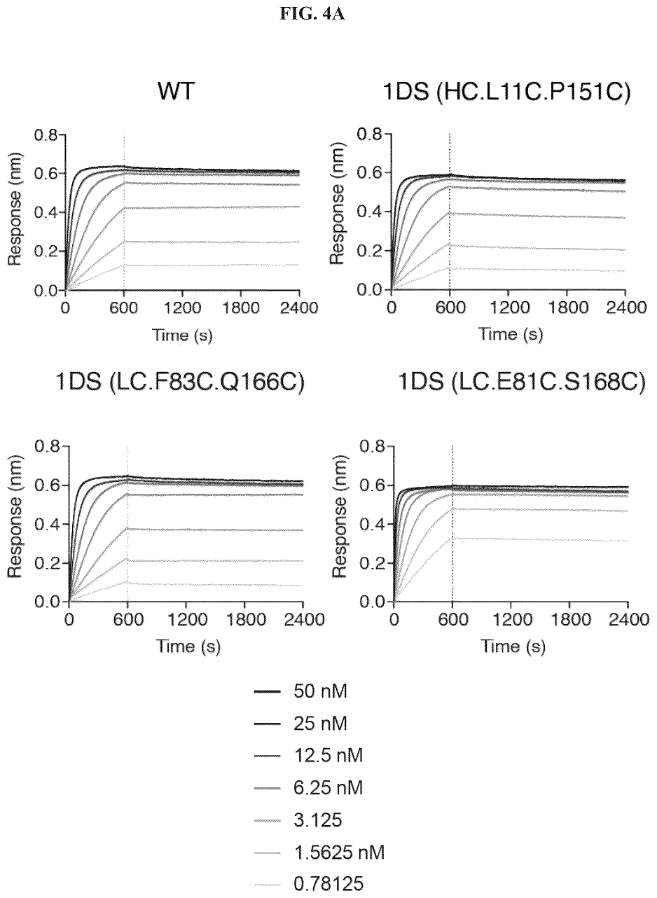

To generate conformationally rigid Fabs, protein engineering solutions that would restrict the flexibility between the variable and constant domains that could be easily transferable between Fabs from different species, frameworks, as well as chimeras, were required. Analysis of structural alignments of existing crystal structures of Fabs from various species and frameworks revealed that rabbit Fabs are naturally less flexible than human or murine Fabs. (FIG. 1A, Table 1). This reduced flexibility is likely due to an interdomain disulfide between residues C80 and C171 (Kabat numbering) in the light chain (LC) elbow. Crystal structures indicated that mutation of the corresponding residues in human Fabs, P80 and S171, to cysteine could allow for disulfide bond formation. Introducing additional disulfide bonds in the elbow region could result in Fabs that are conformationally even more rigid (FIG. 3A). Structures of Fabs in the Protein Data Bank (PDB) were analyzed and additional pairs of conserved residues (E81^{LC}:S68^{LC}, F83^{LC}:Q166^{LC}, I106^{LC}:S171^{LC}, L11^{HC}:P151^{HC}, T110^{HC}:P151^{HC}, anti-Tryptase Fab in 6VVU as reference) that were in close proximity with Cβ-Cβ distance ≤5.5 Å were identified. These were oriented such that cysteine mutations would allow formation of disulfide bonds connecting the variable and constant domains (FIG. 1B).

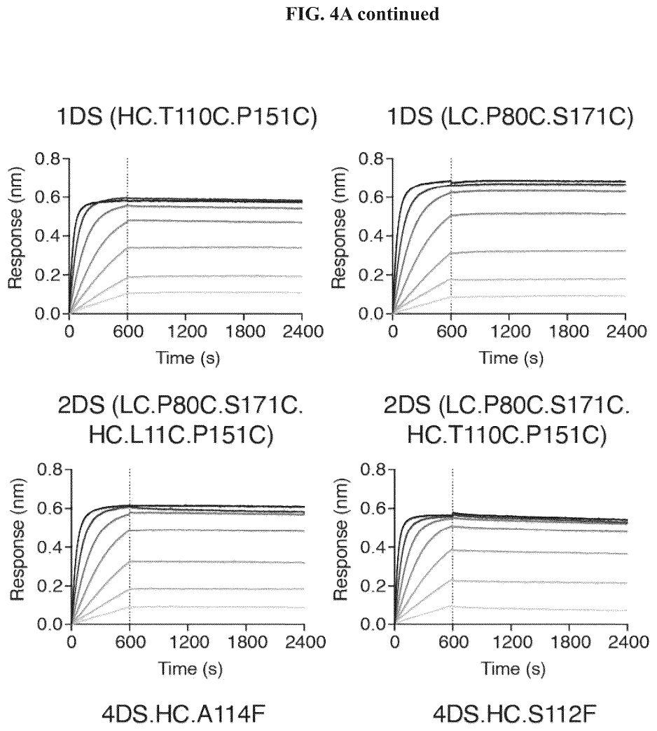

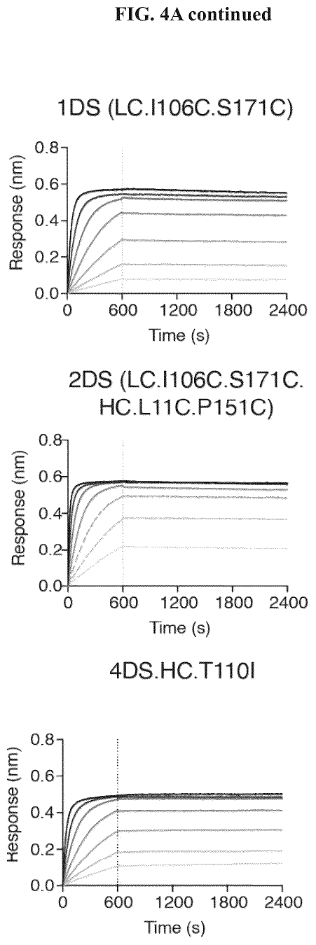

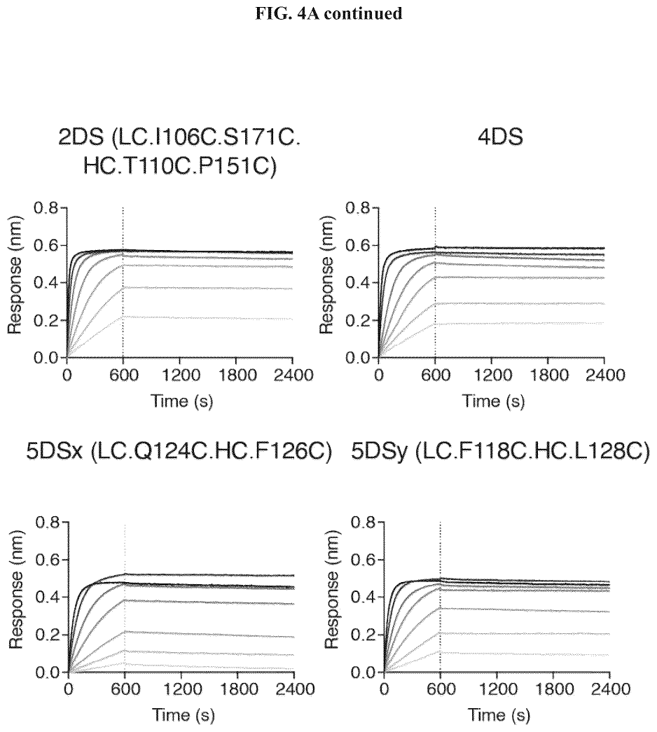

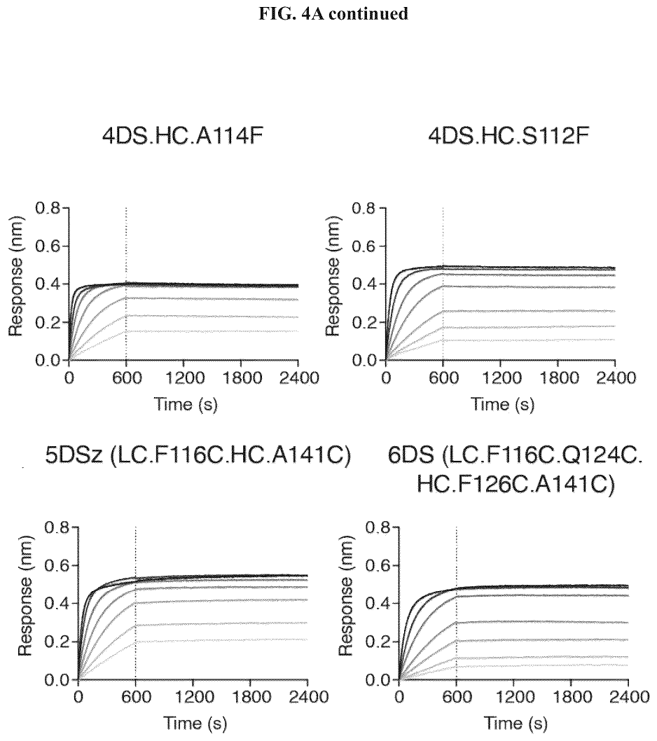

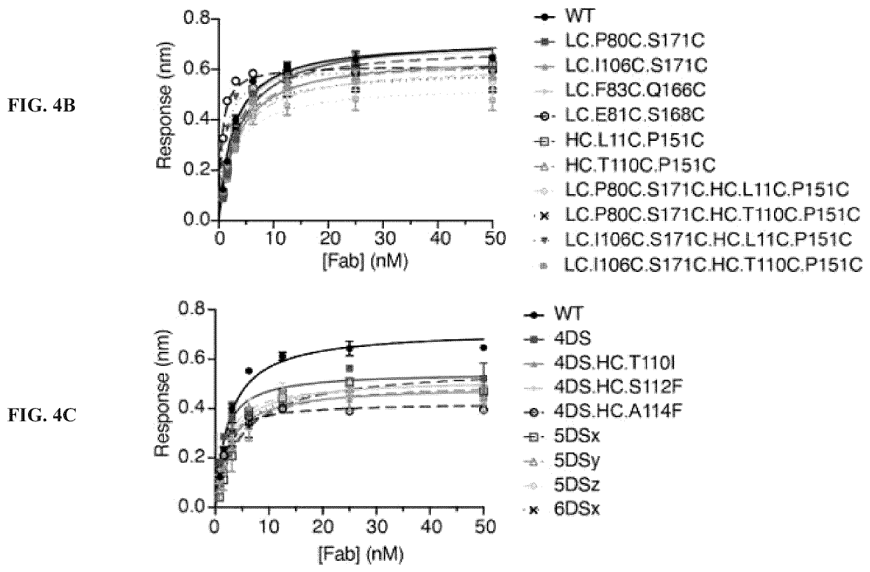

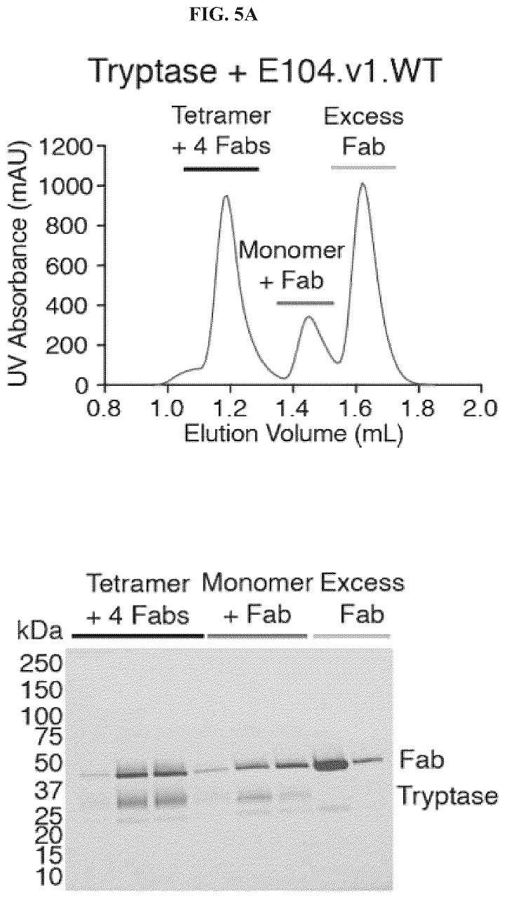

Symmetric tetramer of human β-tryptase (120 kDa) and E104.v1, an anti-tryptase Fab that forms a 4:4, ~320 kDa complex were selected to assess the rigidity of the Fab designs. These mutations were introduced into E104.v1 to generate Fabs that contained an elbow disulfide in their LC, HC, or both. Expression levels of the Fab variants were comparable to wild type (WT), and intact protein LC-MS indicated that each Fab had the expected additional number of disulfide bonds. Biolayer interferometry (BLI) showed that all Fab variants exhibited similar binding affinity and kinetics as E104.v1.WT and formed a 4:4 complex with tryptase (FIGS. 4A-4C). Fitting of the data in FIGS. 4A-4D with a 1:1 binding model was used to calculate the apparent K_{d} values shown in Table A. Table A shows steady state K_{d} values measured in tryptase-Fab BLI experiments.

TABLE A
Steady state K_{d} values measured in tryptase-Fab BLI experiments

| Construct | Steady State Kd (nM) |
|---|---|
| WT | 2.65 |
| 1DS (LC.P80C.S171C) | 3.75 |
| 1DS (LC.I106C.S171C) | 3.41 |
| 1DS (LC.E83C.Q166C) | 2.74 |
| 1DS (LC.E81C.S168C) | 0.55 |
| 1DS (HC.L11C.P151C) | 2.87 |
| 1DS (HC.T110C.P151C) | 2.82 |
| 2DS (LC.P80C.S171C.HC.L11C.P151C) | 3.25 |
| 2DS (LC.P80C.S171C.HC.T110C.P151C) | 2.4 |
| 2DS (LC.I106C.S171C.HC.L11C.P151C) | 1.05 |
| 2DS (LC.I106C.S171C.HC.T110C.P151C) | 2.87 |
| 4DS | 1.63 |
| 4DS.HC.A114F | 1.37 |
| 4DS.HC.T110I | 2.46 |
| 4DS.HC.S112F | 2.59 |
| 5DSx (LC.Q124C.HC.F126C) | 4.81 |
| 5DSy (LC.F118C.HC.L128C) | 3.51 |
| 5DSz (LC.F116C.HC.A141C) | 1.94 |
| 6DS (LC.F116C.Q124C.HC.F126C.A141C) | 3.16 |

TABLE 1
Elbow angles of published rabbit Fab structures¹

| | | | | | LC elbow |
|---|---|---|---|---|---|
| PDB ID | LC | HC | Elbow angle (°) | Method | disulfide |
| 4D3C | L | H | 137.6 | X-ray | no |
| 4HBC | L | H | 144 | X-ray | yes |
| 4HT1 | L | H | 137 | X-ray | no |
| 4JO1 | L | H | 142.7 | X-ray | yes |
| 4JO1 | M | I | 142.9 | X-ray | yes |
| 4JO2 | L | H | 140.8 | X-ray | yes |
| 4JO2 | M | I | 141.9 | X-ray | yes |
| 4JO3 | L | H | 154 | X-ray | yes |
| 4JO3 | M | I | 154.9 | X-ray | yes |
| 4JO4 | M | I | 153.5 | X-ray | yes |
| 4JO4 | L | H | 155.4 | X-ray | yes |
| 4MA3 | A | B | 139.8 | X-ray | no |
| 4MA3 | L | H | 142.6 | X-ray | no |
| 4O4Y | L | H | 144.8 | X-ray | yes |
| 4O51 | A | B | 148.9 | X-ray | no |
| 4O51 | C | D | 149.2 | X-ray | no |
| 4O51 | L | H | 149.5 | X-ray | no |
| 4O51 | E | F | 149.6 | X-ray | no |
| 4ZTO | M | I | 141.7 | X-ray | yes |
| 4ZTO | L | H | 143.6 | X-ray | yes |
| 4ZTP | L | H | 151.2 | X-ray | yes |
| 5C0N | D | C | 137.9 | X-ray | yes |
| 5C0N | L | H | 139.3 | X-ray | yes |
| 5DMG | D | C | 145.9 | X-ray | no |
| 5DMG | F | E | 151.3 | X-ray | no |
| 5DMG | L | H | 155 | X-ray | no |
| 5DRN | B | A | 150.3 | X-ray | yes |
| 5DRN | L | H | 150.8 | X-ray | yes |
| 5DS8 | L | H | 146.7 | X-ray | yes |
| 5DS8 | B | A | 156.4 | X-ray | yes |
| 5DSC | B | A | 148.7 | X-ray | yes |
| 5DSC | D | C | 148.8 | X-ray | yes |
| 5DSC | F | E | 149.7 | X-ray | yes |
| 5DSC | L | H | 151.5 | X-ray | yes |
| 5DTF | B | A | 153 | X-ray | yes |
| 5DTF | L | H | 154.4 | X-ray | yes |
| 5DUB | L | H | 146.7 | X-ray | yes |
| 5DUB | B | A | 156.5 | X-ray | yes |
| 5M63 | N | M | 143.3 | X-ray | yes |
| 5M63 | L | H | 143.4 | X-ray | yes |
| 5TH9 | L | H | 137.1 | X-ray | no |
| 5TH9 | M | I | 137.8 | X-ray | no |
| 5V6L | L | H | 139.2 | X-ray | yes |
| 5V6L | M | I | 149.1 | X-ray | yes |
| 5V6M | L | H | 140.5 | X-ray | no |
| 6CEZ | L | H | 138 | X-ray | yes |
| 6CJK | C | D | 146.1 | X-ray | yes |
| 6CJK | A | B | 149.4 | X-ray | yes |
| 6CWT | D | C | 140.7 | X-ray | no |
| 6CWT | B | A | 141.1 | X-ray | no |
| 6DID | H | L | 145.6 | EM | yes |
| 6I9I | B | A | 151.4 | X-ray | yes |
| 6I9I | L | H | 152.5 | X-ray | yes |
| 6LDV | L | H | 142.5 | X-ray | yes |
| 6LDW | A | B | 139.8 | X-ray | yes |
| 6LDW | L | H | 140.5 | X-ray | yes |
| 6LDX | L | H | 146.2 | X-ray | yes |
| 6LDY | L | H | 148.6 | X-ray | yes |
| 6LDY | B | A | 151.6 | X-ray | yes |
| 6T3F | L | H | 143.4 | X-ray | yes |
| 6X1S | F | E | 150.2 | X-ray | yes |
| 6X1S | D | C | 151.3 | X-ray | yes |
| 6X1S | L | H | 158.5 | X-ray | yes |
| 6X1S | B | A | 159.1 | X-ray | yes |
| 6X1T | D | C | 145.9 | X-ray | yes |
| 6X1T | K | J | 146.1 | X-ray | yes |
| 6X1T | B | A | 146.9 | X-ray | yes |
| 6X1T | L | H | 148.9 | X-ray | yes |
| 6X1T | F | E | 150.7 | X-ray | yes |
| 6X1T | I | G | 153.9 | X-ray | yes |
| 6X1U | L | H | 144.8 | X-ray | yes |
| 6X1U | B | A | 145.6 | X-ray | yes |
| 6X1V | L | H | 142.3 | X-ray | yes |
| 6X1W | L | H | 150.7 | X-ray | yes |
| 7D9Z | L | H | 144.7 | X-ray | yes |
| 7DCE | L | H | 144.7 | EM | yes |
| 7MFR | A | B | 148.7 | X-ray | no |
| 7MFR | X | Y | 148.7 | X-ray | no |
| 7NKS | D | C | 146.1 | X-ray | yes |
| 7NKS | L | H | 146.7 | X-ray | yes |
| 7NRH | L | H | 146.7 | EM | yes |
| 7O9S | L | H | 150.8 | X-ray | yes |
| 7RA7 | L | H | 147.3 | X-ray | yes |
| 7RA7 | B | A | 155.3 | X-ray | yes |
| 8DF1 | R | Q | 146.9 | X-ray | yes |
| 8DF1 | N | M | 147.7 | X-ray | yes |
| 8DF1 | V | U | 147.8 | X-ray | yes |
| 8DF1 | P | O | 148.3 | X-ray | yes |
| 8DF1 | L | H | 148.7 | X-ray | yes |

¹Elbow angles were calculated using http://linum.proteinmodel.org/AS2TS/RBOW/index.html. LC and HC indicate chain IDs for the light and heavy chains, respectively.

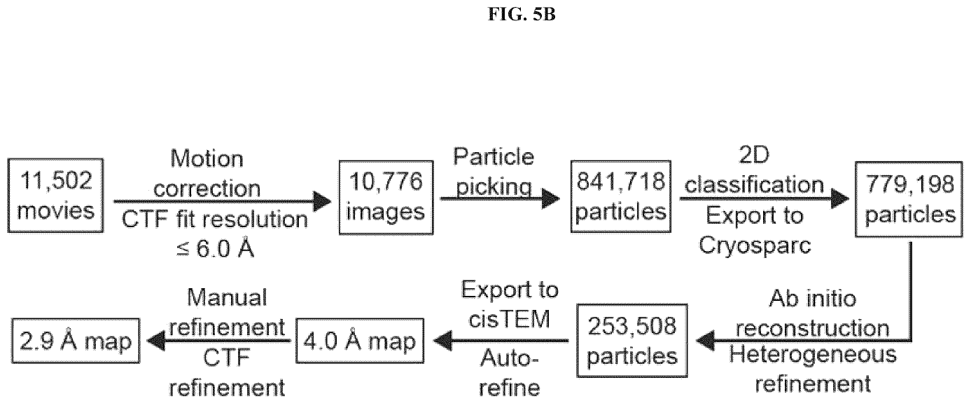

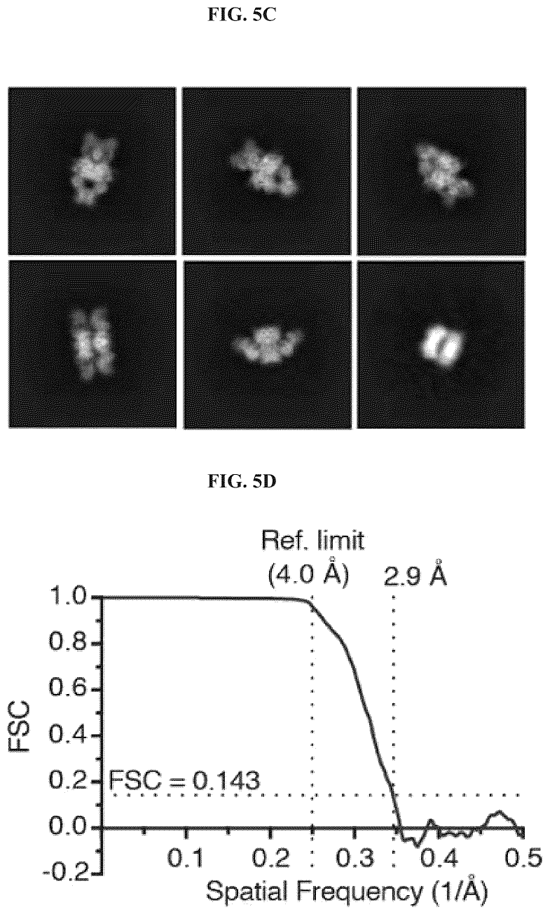

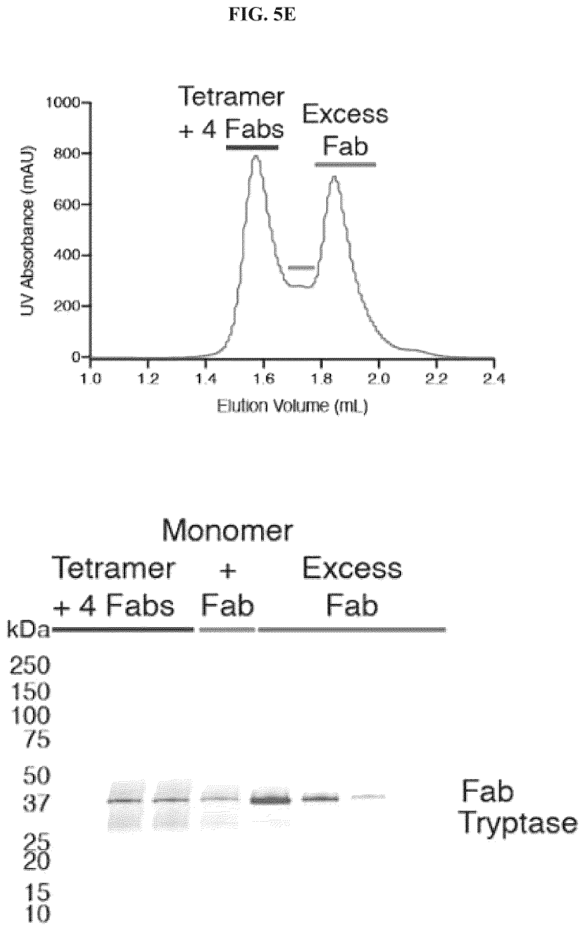

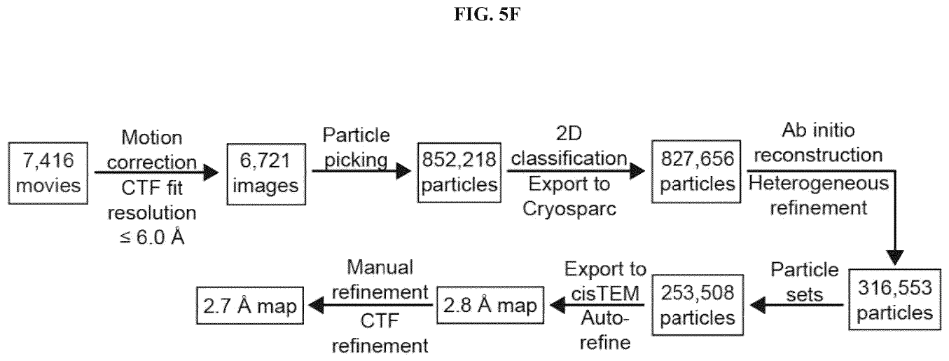

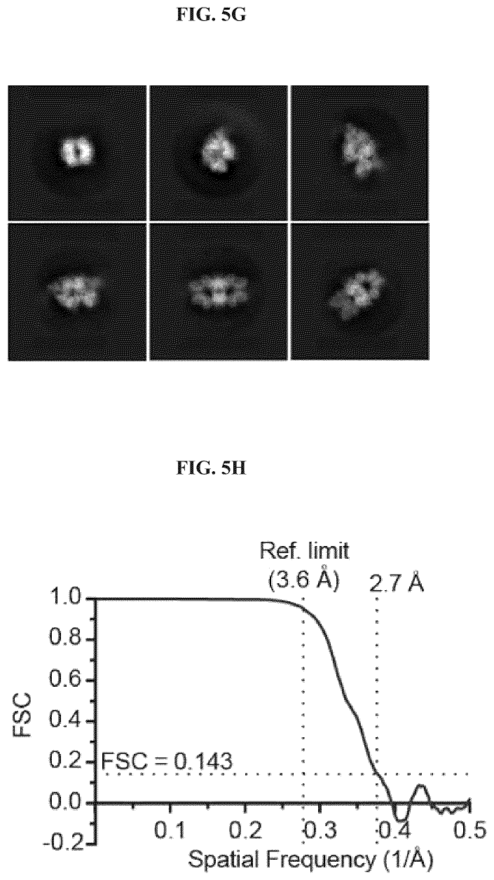

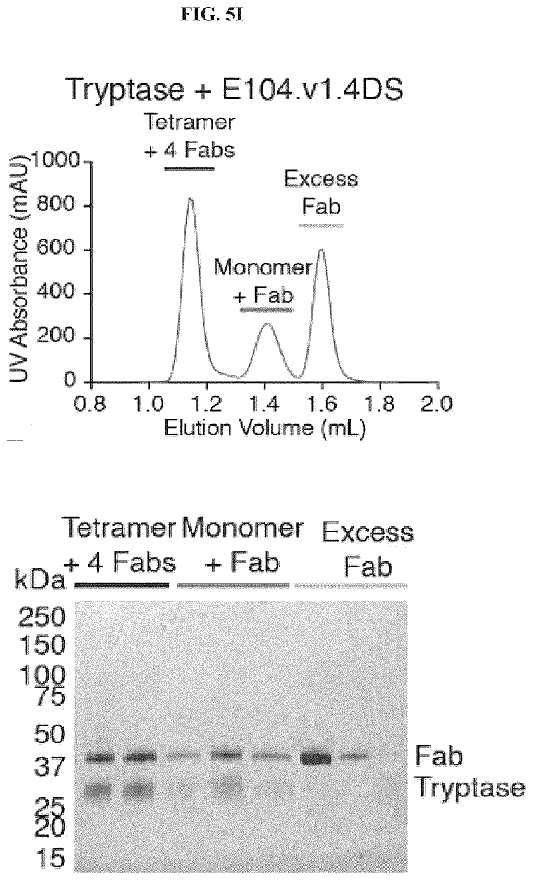

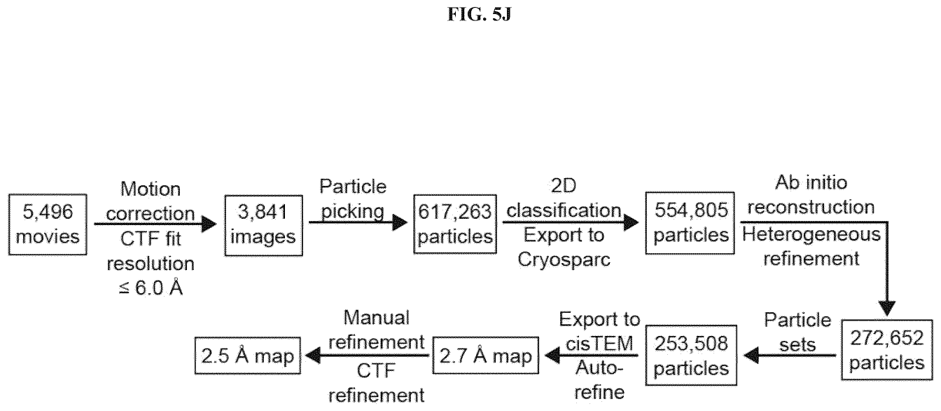

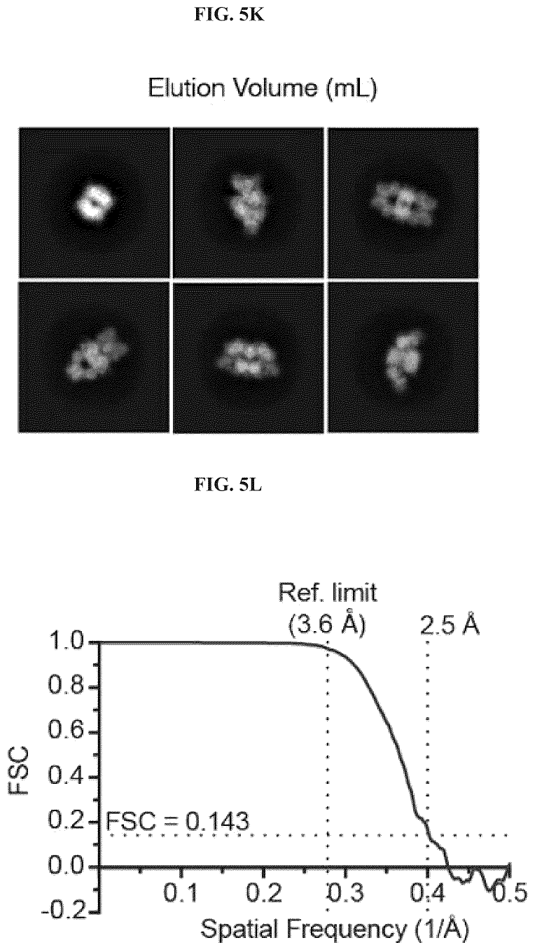

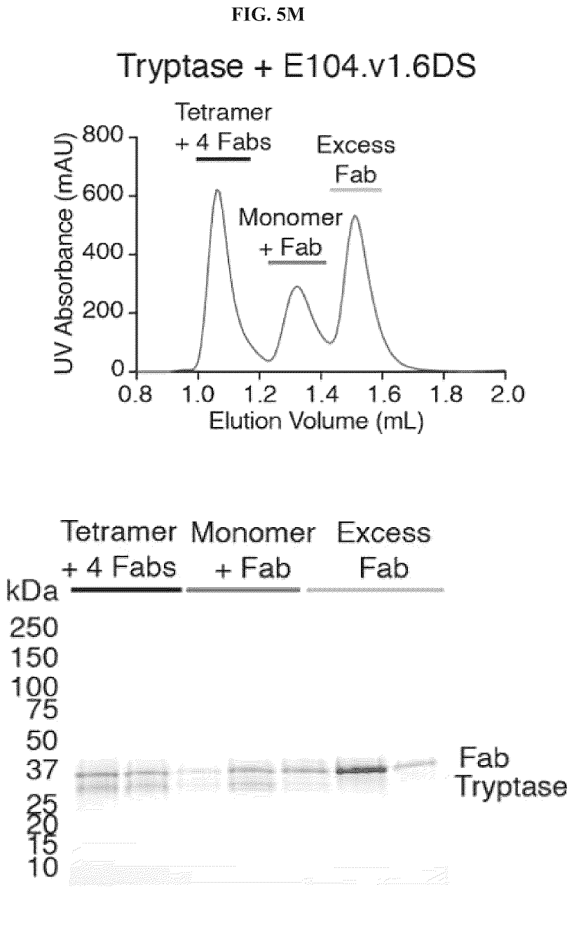

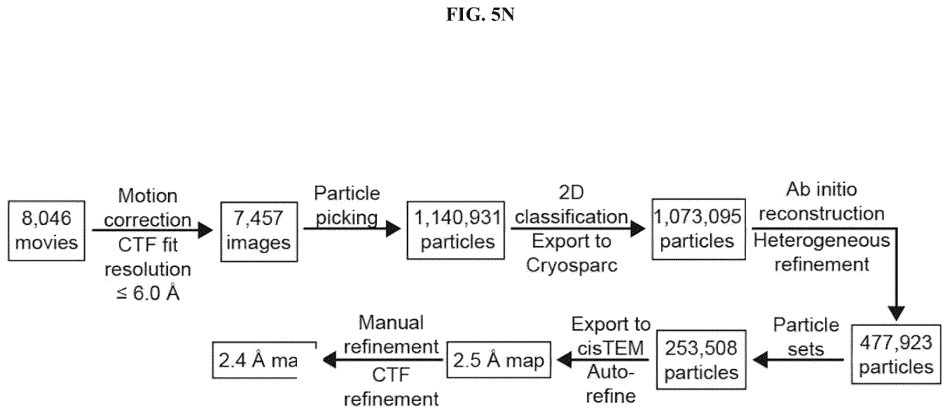

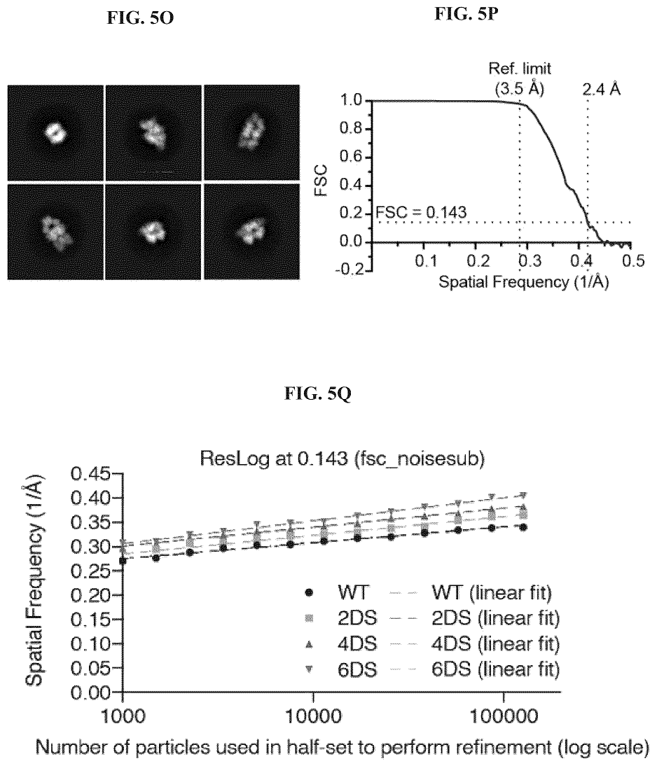

Cryo-EM datasets were collected for samples of the tryptase tetramer in complex with E104.v1.WT or with a construct containing two elbow disulfides (2DS) formed by variants L11C^{HC}:P151C^{HC} and P80C^{LC}:S171C^{LC}. For the WT dataset, a reconstruction with a resolution of 2.9 Å (FIG. 3C, FIGS. 5A-E) was obtained. The tryptase tetramer was the best-resolved part of the structure, and the Fabs were relatively poorly resolved, especially in the constant domains, consistent with the expected flexibility of WT Fab. From the 2DS dataset, a reconstruction was obtained with an improved resolution of 2.7 Å (FIG. 3D, FIGS. 5F-J) and higher local resolution in tryptase, as well as the Fab variable and constant domains, compared to the WT structure, indicating that the 2DS Fab was indeed less flexible than the WT Fab (FIGS. 3C-D, 3G-H, 3K-L).

Though the 2DS Fab was more rigid than the WT Fab, the constant domains of the 2DS Fab were still less well-resolved than the variable domains, indicating some flexibility remained in the 2DS Fab elbow. To generate Fabs that are more rigid, additional sites were identified in the Fab LC and HC elbows to introduce more disulfides to further constrain the conformation of the Fab (FIG. 3A, FIG. 1C). The structure of the E104.v1.WT-tryptase complex (PDB code 6VVU) indicated that the mutations P40C^{LC}:E165C^{LC} could lead to disulfide formation. L108^{HC} and P153^{HC} were too far apart (Cβ-Cβ distance=6.3 Å) to allow for disulfide formation with L108C^{HC}:P153C^{HC} mutations. Since P153^{HC} is in a loop, a cysteine residue between E152^{HC} and P153^{HC} was inserted to lengthen the loop and position the new cysteine close enough to enable disulfide bond formation with L108C^{HC}. This design, 4DS, contained a total of four intrachain disulfides in the elbow region, two in each chain (FIG. 3A). As with the 2DS Fabs, the various 4DS Fabs expressed at levels similar to E104.v1.WT, possessed all four engineered elbow disulfides as indicated by intact LC-MS analysis, and bound to tryptase with similar binding affinity and kinetics as E104.v1.WT (FIGS. 4A-4C, Table A). To further characterize these Fabs, structures of the 4DS Fab variants were crystallized and solved at a resolution of 2.0 Å (FIGS. 1E-F). The structures confirmed that all four elbow disulfides formed as designed. To determine whether the additional elbow disulfides in the 4DS Fab would further reduce flexibility, a cryo-EM dataset was collected for the tryptase-4DS Fab complex, resulting in a 3D reconstruction with a resolution of 2.5 Å (FIG. 3E, FIGS. 5K-0). Strikingly, in addition to the improved overall resolution, there were notable improvements in local resolution throughout the structure (FIGS. 3E, 3I, 3M), especially in the constant domains of the Fabs, demonstrating that the new disulfides in the 4DS Fab significantly increased the rigidity of the Fab.

It was observed that the C-termini of the constant domains of the 4DS Fabs still retained some flexibility relative to the rest of the Fab (FIG. 3E). In an effort to engineer this region of the Fab to further decrease flexibility, the 4DS Fab crystal structures were examined and additional sites in the constant domain where interchain disulfides could be introduced between the LC and HC were identified (FIG. 1D). Variants of E104.v1 that contained pairs of cysteine mutations at one or more of these sites in combination with the 4DS elbow mutations were designed and expressed. All of the 5DS/6DS Fabs bound to tryptase with similar binding affinity and kinetics as E104.v1.WT (FIGS. 4A-4C, Table A), demonstrating that the constant domain mutations do not negatively affect the ability of the Fab to bind antigen. Furthermore, crystal structures of a 5DS (F116C^{LC}:A141C^{HC}) and a 6DS (F116C^{LC}:A141C^{HC} and Q124C^{LC}:F126C^{HC}) Fab further confirmed proper formation of all engineered disulfides (FIG. 1G and FIG. 3B). A cryo-EM dataset was collected for the tryptase-6DS Fab complex, resulting in a 3D reconstruction with an overall resolution of 2.4 Å (FIG. 3F, FIGS. 5P-T). Overall, the 6DS map was very similar to the 4DS map with some improvements in local resolution, especially at the C-termini of the constant domains, consistent with the interchain disulfides in the constant domain increasing the rigidity of that domain (FIGS. 3F, 3J, 3N). Comparison of ResLog plots for each of the tryptase-Fab complexes further demonstrated that each additional pair of engineered disulfides in the Fab led to marked improvements in the quality of the data, while also reducing the number of particles required to achieve higher resolutions (FIG. 5U).

### Modularity of Rigid Fab Designs

The murine Fab 7A9 and the human-mouse chimeric Fab fragment from Rituximab (RTX), which target the membrane proteins Nav1.7 and CD20, respectively, were selected for further engineering to evaluate whether the same Rigid Fab designs used for E104.v1 could be applied to rigidify Fabs derived from other species or chimeras. Previous cryo-EM structures of these WT Fabs bound to Nav1.7 and CD20 revealed inherent flexibility in the Fabs. (See, Xu et al, Cell, 176:702-715.e14, 2019 and Rouge et al, Science, 367:1224-1230, 2020.

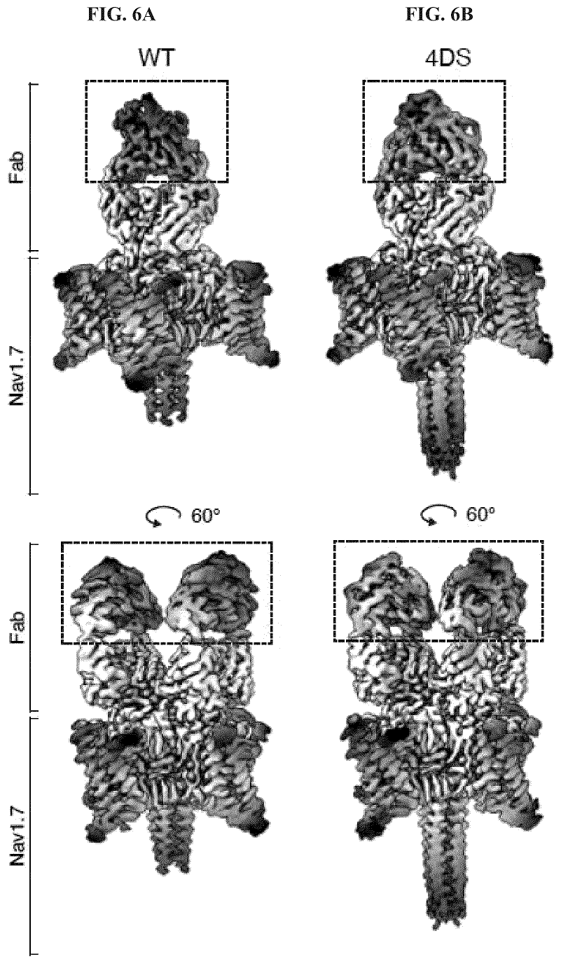

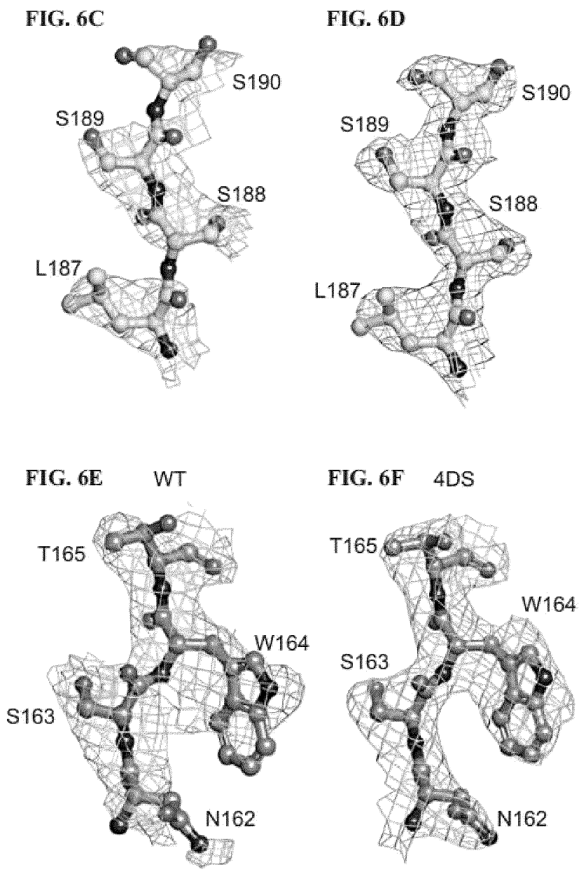

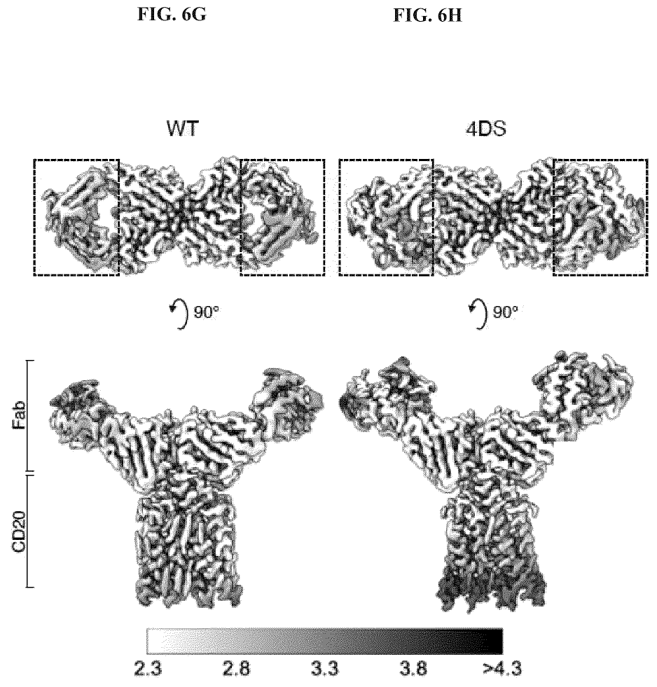

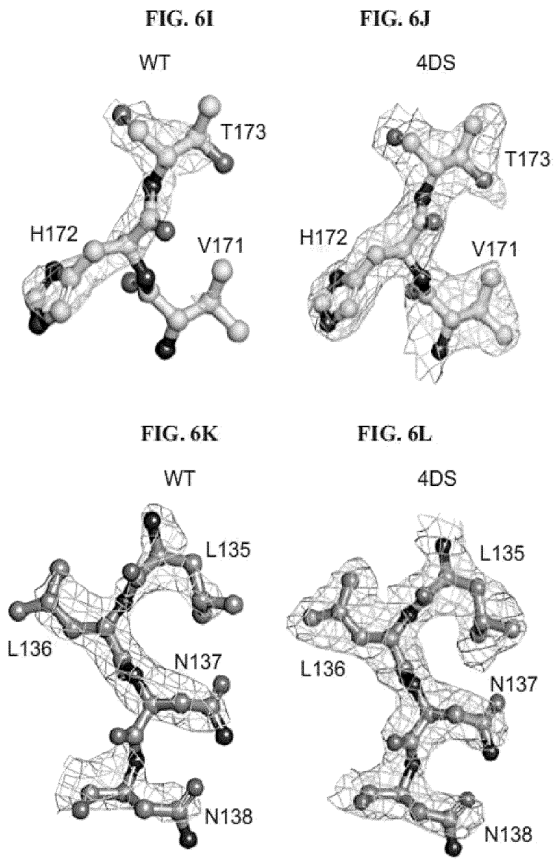

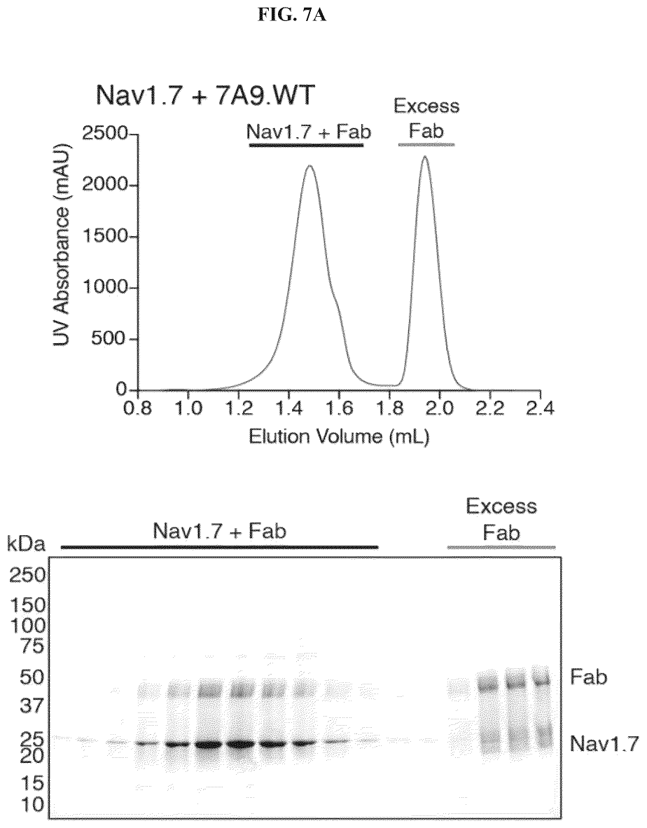

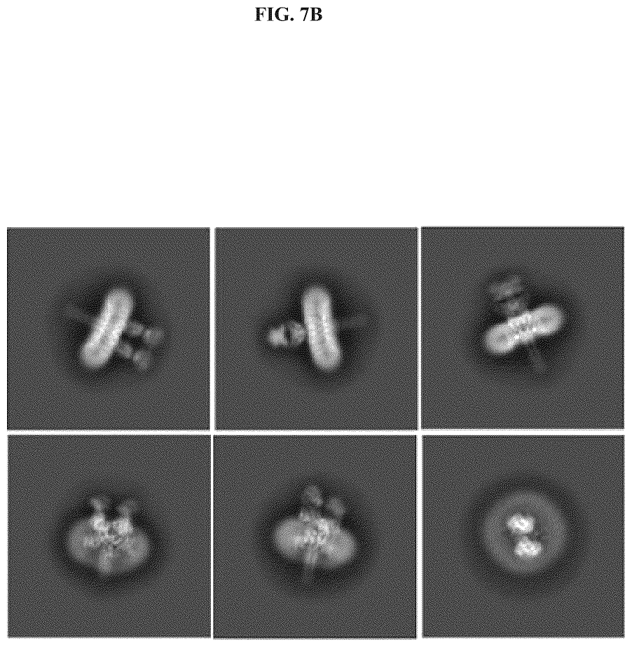

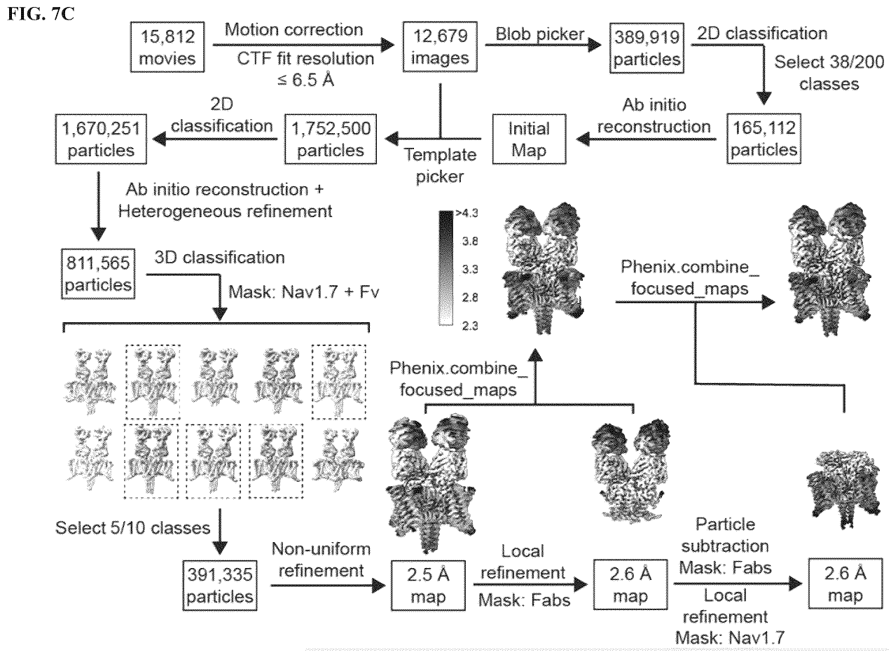

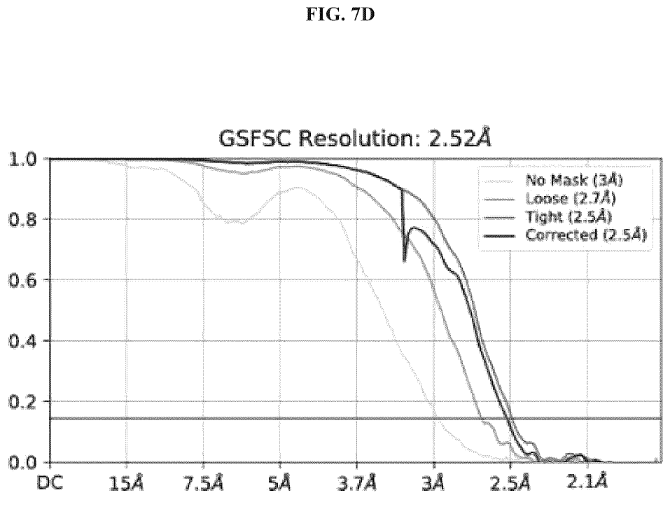

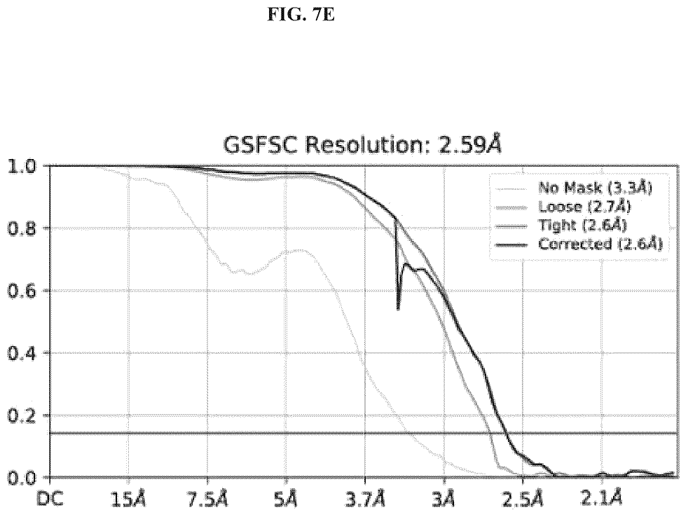

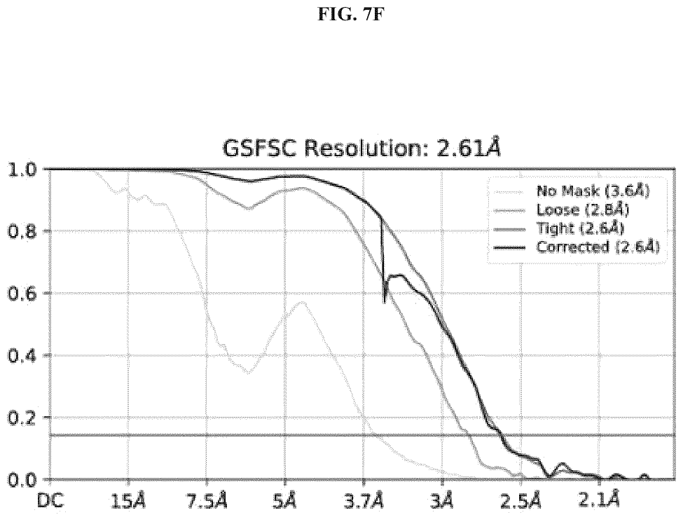

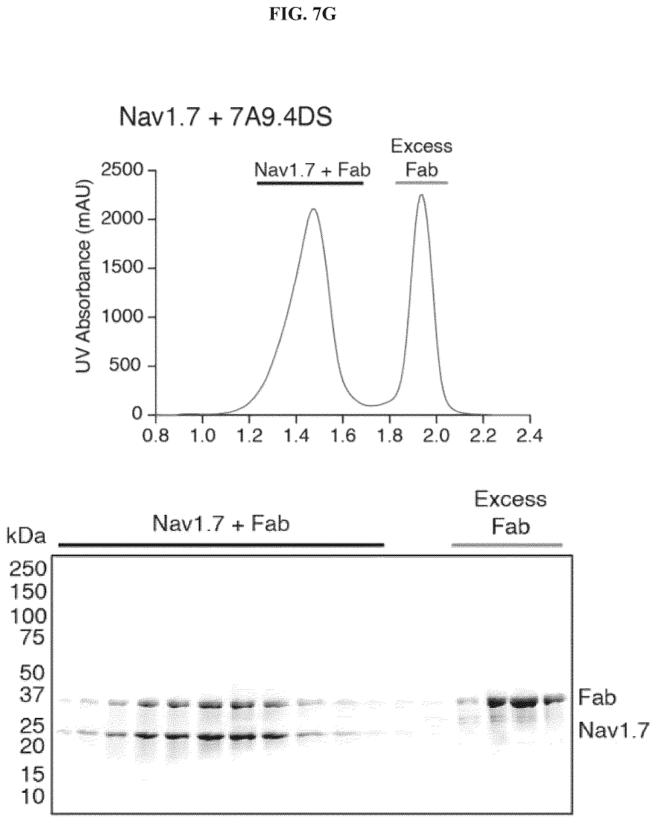

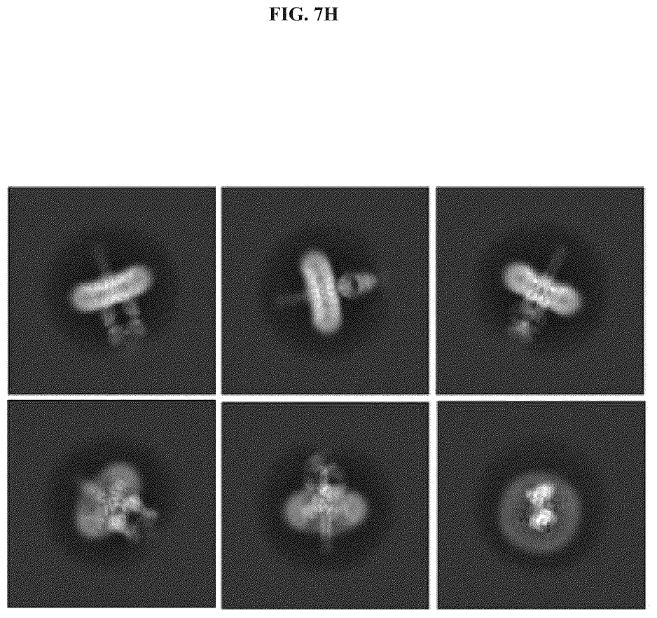

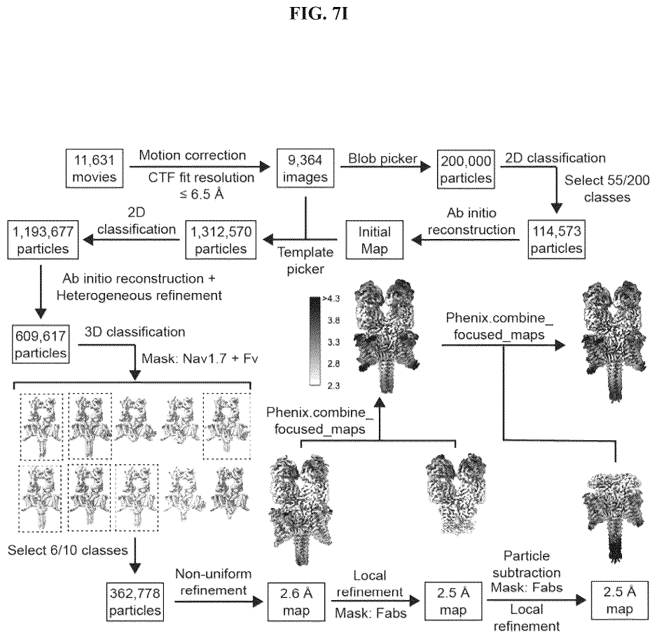

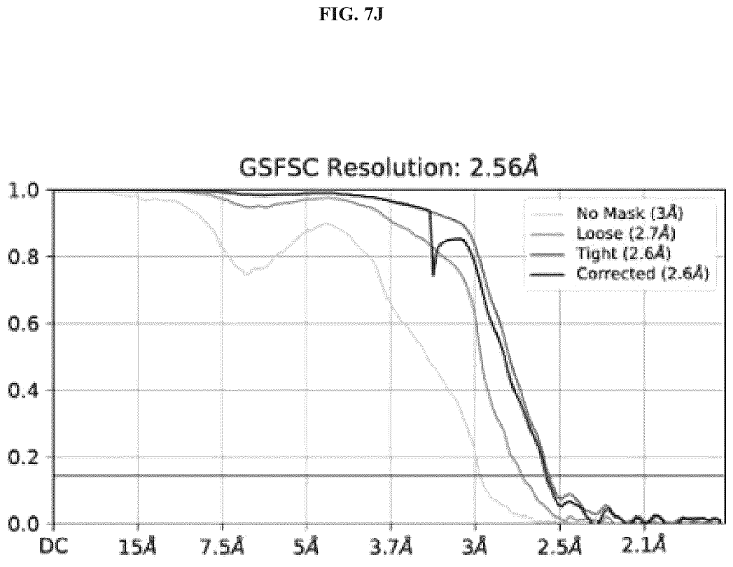

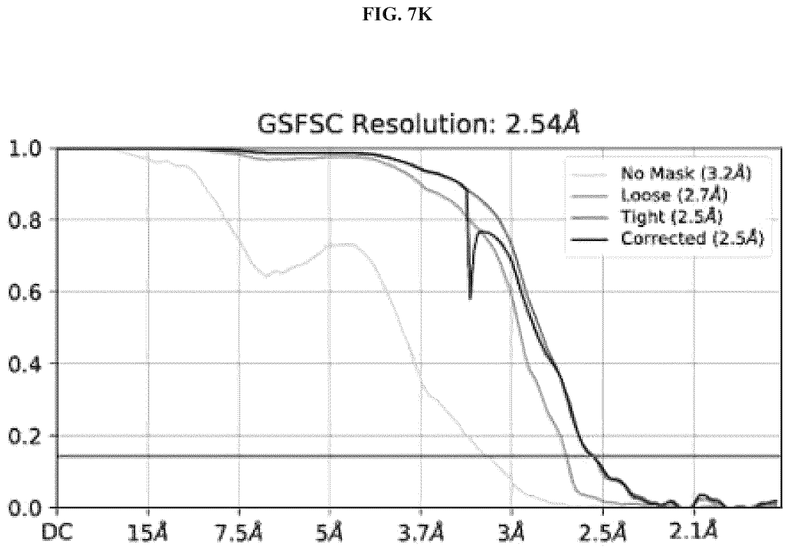

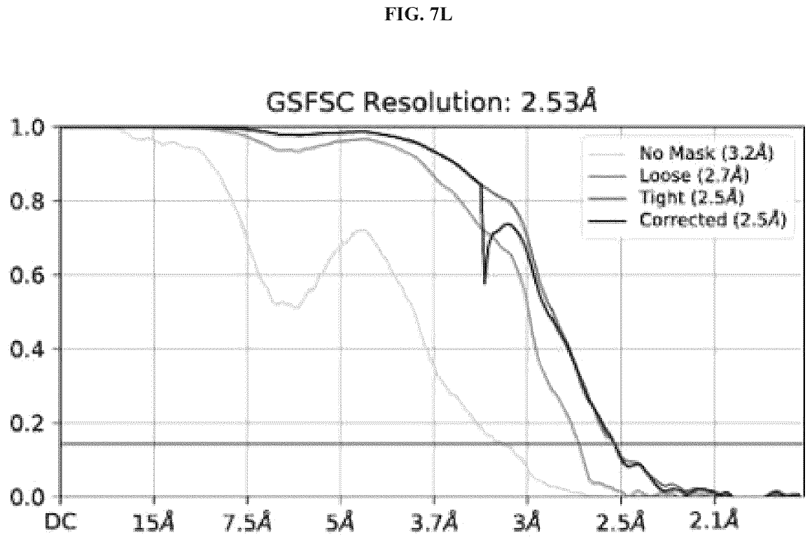

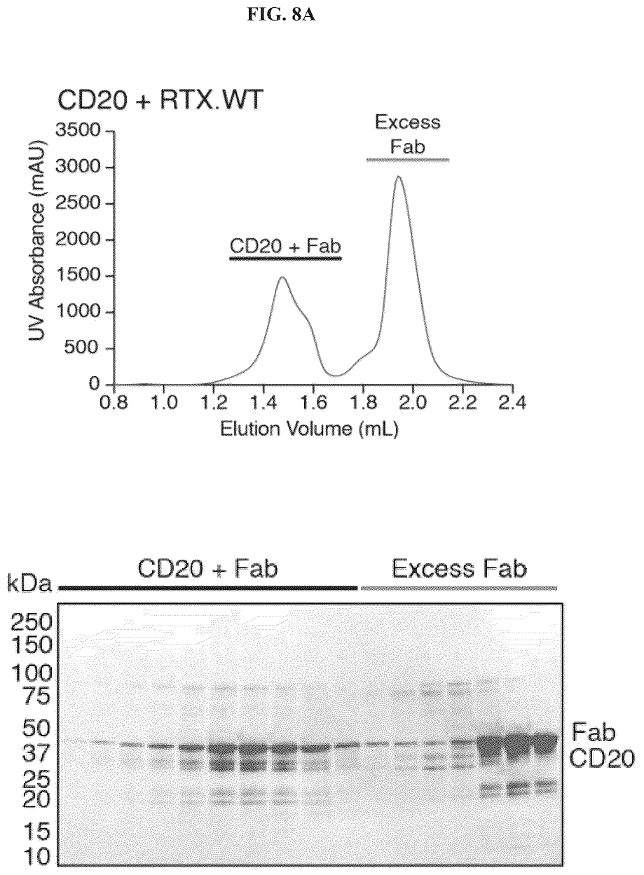

4DS variants of both 7A9 and RTX Fabs were generated using cysteine mutations at the same exact positions (Kabat numbering) as those introduced in the E104.v1.4DS Fab. Intact LC/MS analysis indicated that all four elbow disulfides were present in both 7A9.4DS and RTX.4DS Fabs. Cryo-EM datasets were collected for each antigen bound to either the WT Fab or 4DS Fab (FIGS. 6-8). For Nav1.7, which forms a 4:2 Nav1.7:Fab complex, the overall resolution of the consensus 3D reconstructions for both the Nav1.7-7A9.WT and Nav1.7-7A9.4DS complexes was 2.6 Å (FIGS. 6A-B, FIG. 7). Local refinement with a mask around the Fabs in the 7A9.4DS complex led to greater improvements in local resolution compared to 7A9.WT, especially in the constant domain, consistent with 7A9. 4DS having increased rigidity (FIGS. 6A-F, FIG. 7).

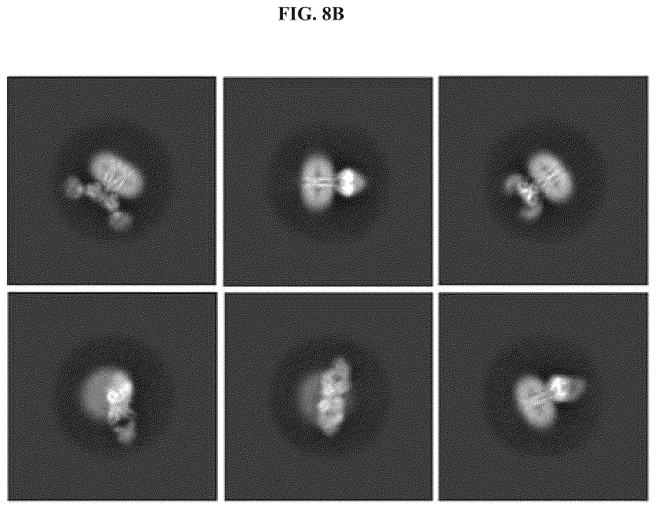

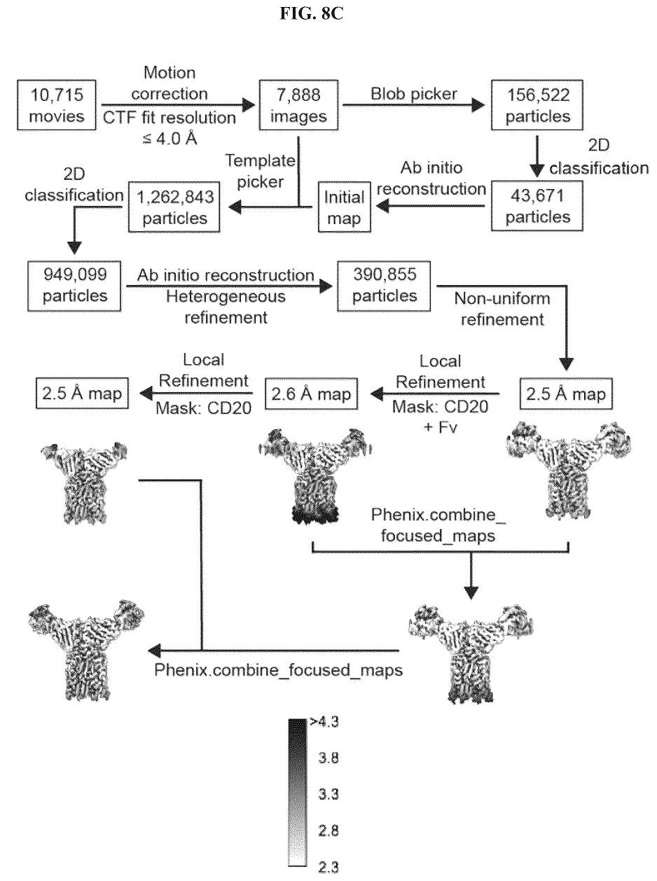

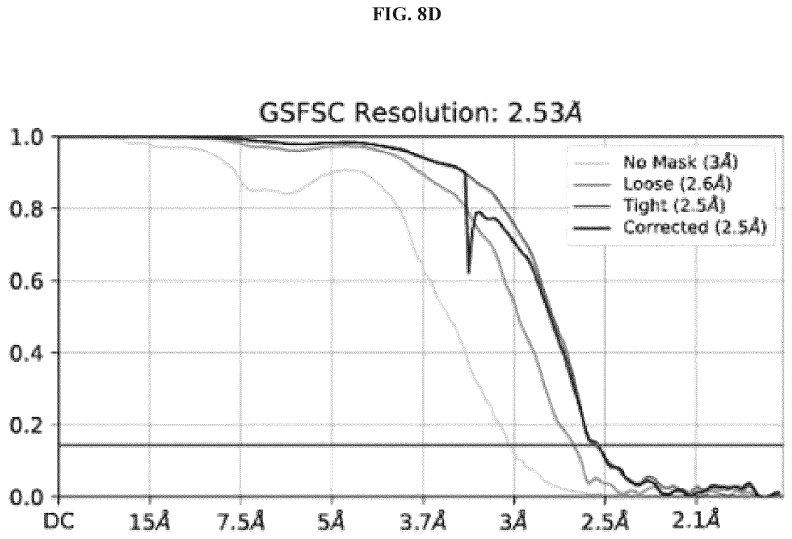

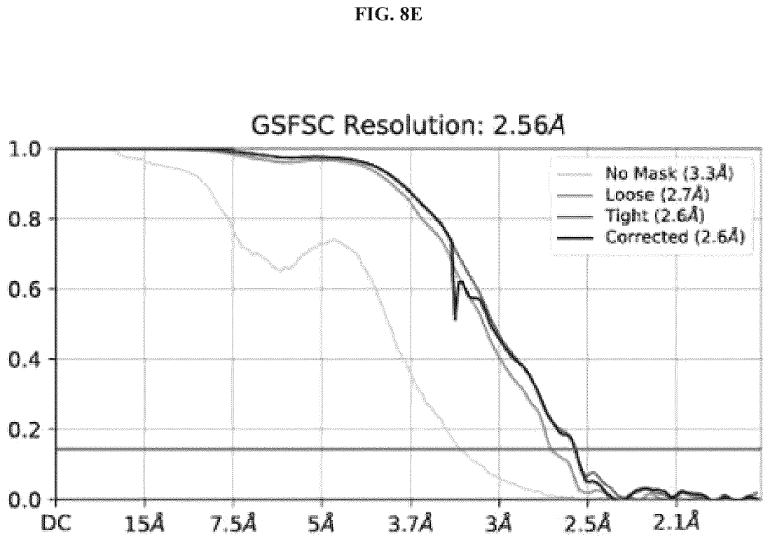

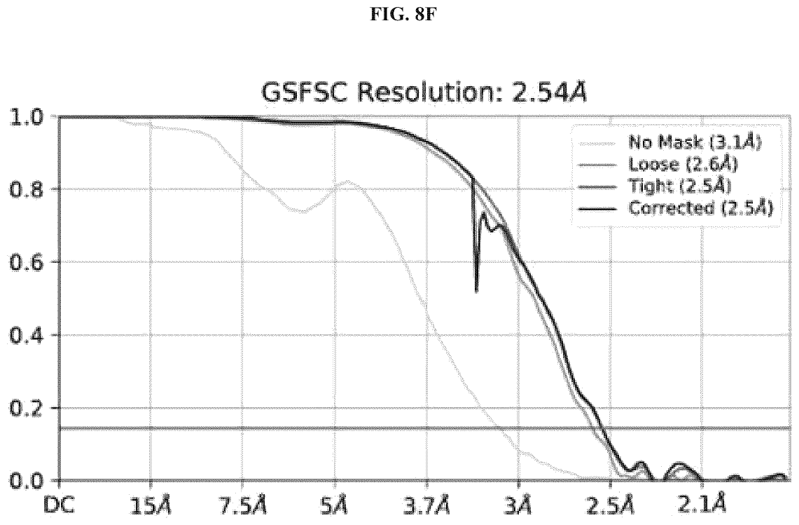

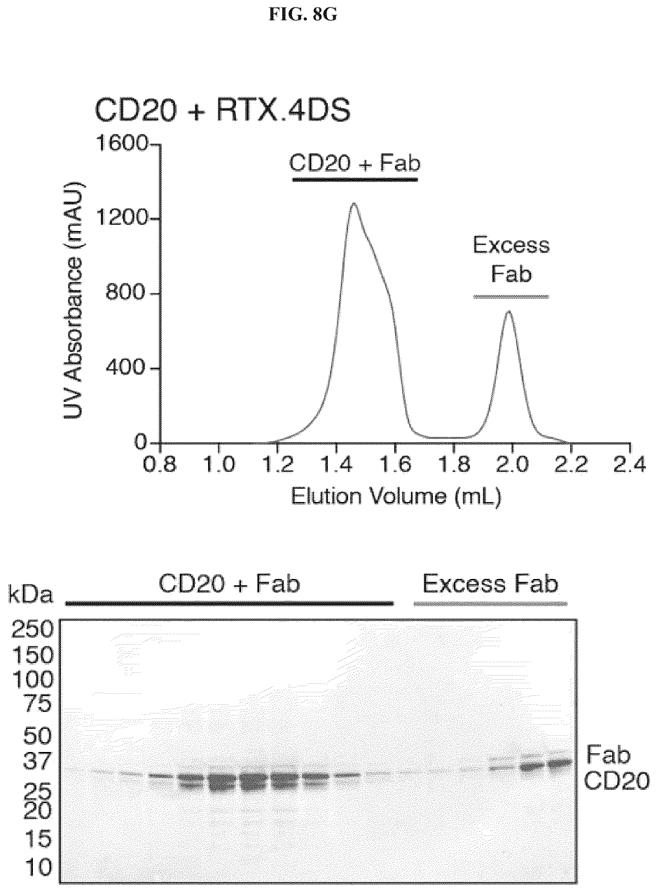

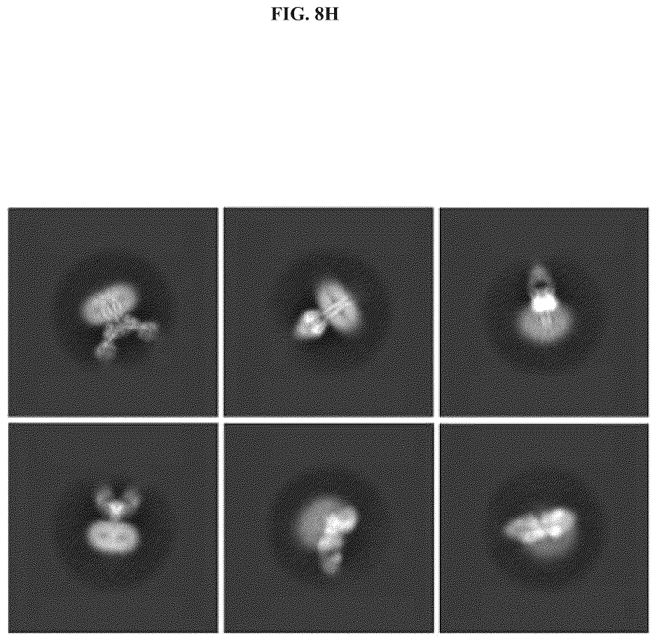

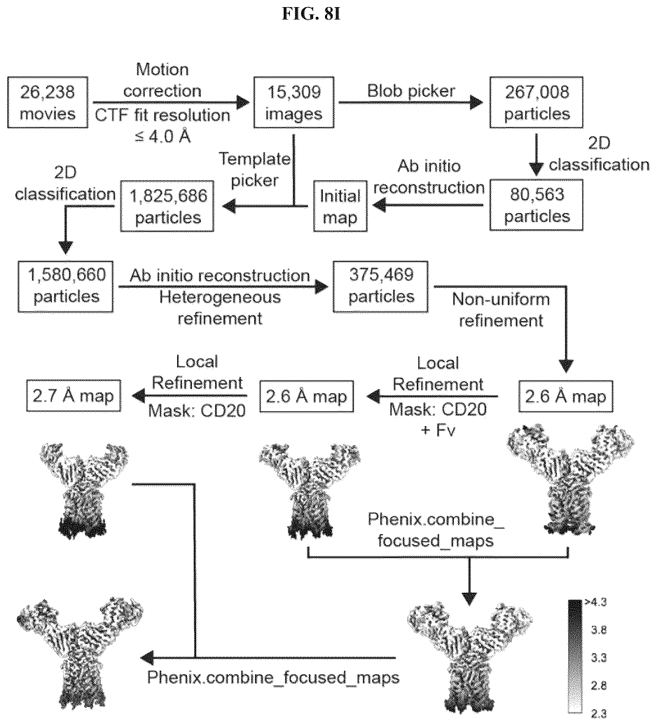

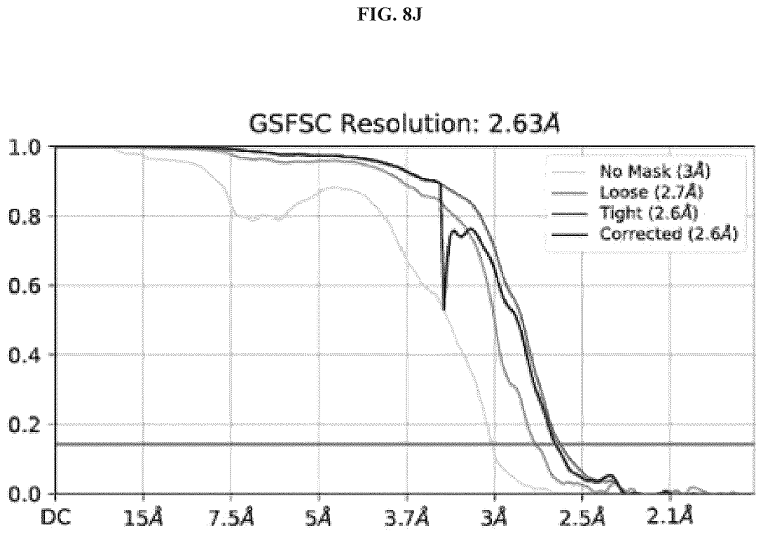

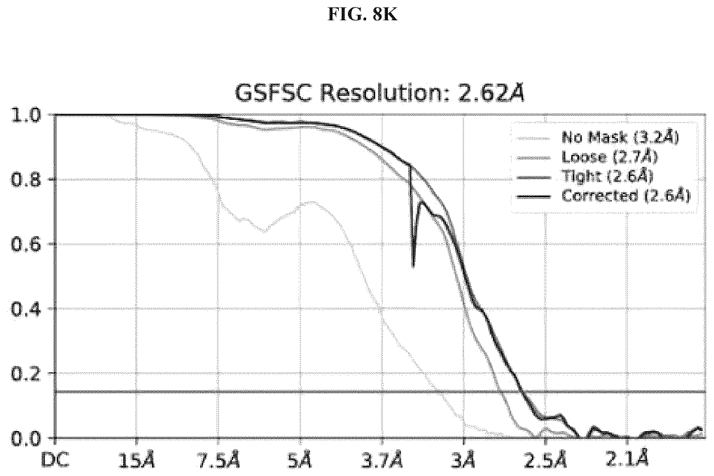

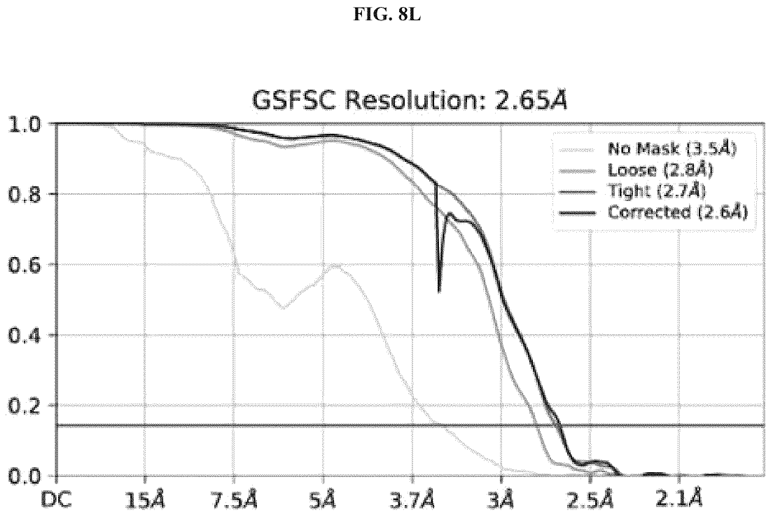

For CD20, which forms a 2:2 CD20-RTX Fab complex, the overall resolution of the consensus 3D reconstructions for both the CD20-RTX.WT and CD20-RTX.4DS complexes was 2.6 Å (FIGS. 6G-H, FIG. 8). The constant domains of the 4DS Fabs were resolved to a much higher resolution than the WT Fabs, confirming that the 4DS mutations did indeed render the Fab significantly more rigid (FIGS. 6G-L). This increased rigidity of RTX.4DS appeared to lead to lower resolution of the portions of the CD20 transmembrane helices distal to the Fab binding site, compared to the map obtained for the RTX.WT complex (FIG. 8). This effect may have been in part due to the nature of the CD20-RTX complex, in which two Fabs bind to one side of the CD20 dimer and to each other. The rigidity and greater mass of the 4DS Fabs, combined with the relative intrinsic flexibility of CD20 and added alignment noise from the detergent micelle surrounding it, likely caused the Fabs to become very well aligned at the cost of alignment accuracy and resolution in the regions of CD20 distal from the Fabs during refinement. In the case of the WT complex, the flexibility of the Fabs caused the constant domains to have less weight in determining particle alignments, allowing for improved alignments for CD20 compared to the 4DS Fab complex. Iterative local refinements using masks to exclude the 4DS Fab constant domains, followed by exclusion of the entire Fab, led to significant improvements in the resolution of the intracellular side of the CD20 helices (FIG. 8L). The consensus map and focused maps were combined to generate a composite map that had improved resolution at the Fab-CD20 interface, as well as throughout CD20. The same procedure was applied to the RTX.WT dataset, leading to a final composite map with similar overall resolution to the RTX.4DS composite map (FIGS. 6G-H, FIG. 8). Thus, even though the rigidity of the 4DS Fab initially led to lower resolution of the antigen, local refinements were able to ameliorate this effect. While this effect was not ideal for solving a structure of this particular antigen, the observation that the RTX.4DS Fab could so strongly drive particle alignments underscored the marked increase in rigidity of 4DS Fab variants relative to WT Fabs.

Overall, the increased rigidity observed for both the 4DS variants of the 7A9 and RTX Fabs demonstrated the modularity of the 4DS Rigid Fab design. The same set of mutations introduced in the humanized E104.v1.4DS Fab successfully resulted in conformationally rigid Fabs against other antigens, even for murine or chimeric Fabs like 7A9 and RTX, respectively.

### Rigid Fabs Enable High-Resolution Structures of Small Proteins

To date, there are no cryo-EM structures of Fabs in complex with monomeric antigens smaller than 50 kDa in the EMDB with resolutions better than ~3.0 Å and only 7 unique monomeric antigen-Fab complexes with resolutions better than ~3.5 Å, underscoring the difficulty of using flexible WT Fabs to solve high-resolution structures of small antigens. In addition, there are no structures currently in the EMDB of monomeric antigens smaller than 50 kDa bound to any structure chaperone that have resolutions better than ~3.0 Å. It was determined whether Rigid Fabs, given their moderate size, were sufficient to enable high resolution structures of small antigens. To test this, the cytokine Ang2 (26 kDa), an extracellular protein and the GTPase KRAS (21 kDa), an intracellular protein, both of which are monomeric, were selected as targets, and a 6DS variant of the anti-Ang2 Fab 5 Å12 and a 4DS variant of the anti-KRAS Fab 2H11 were generated based on the designs for the 4DS and 6DS variants of the anti-tryptase Fab E104.v1.

### High-Resolution Cryo-EM Structure of a 26 kDa Cytokine

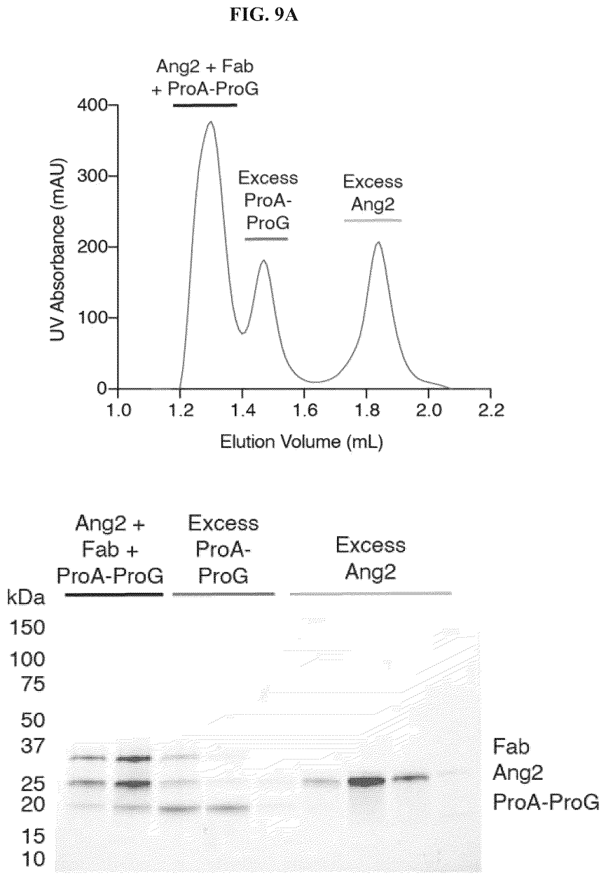

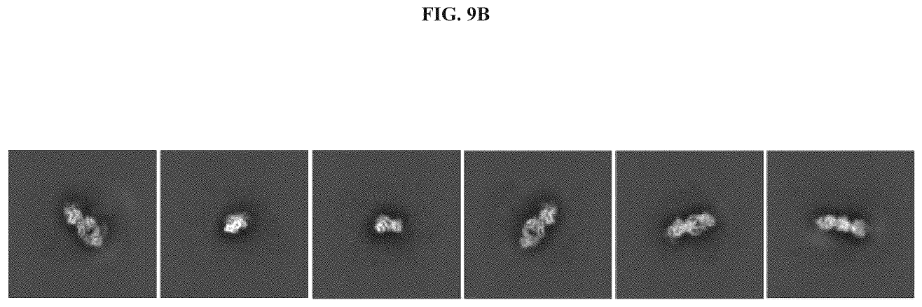

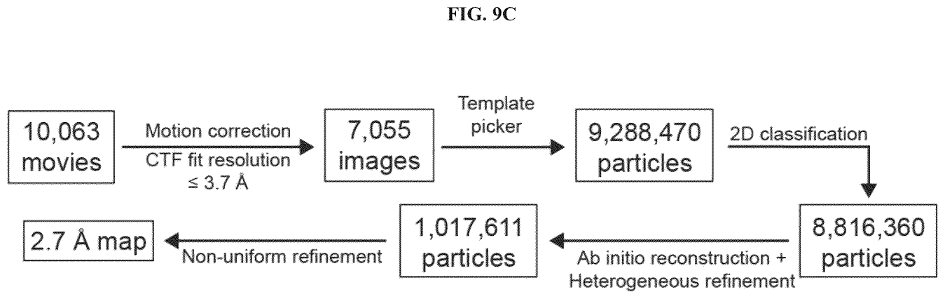

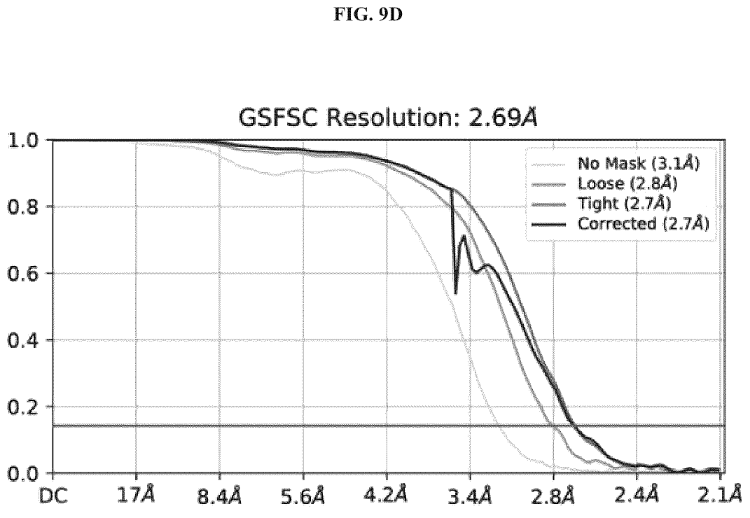

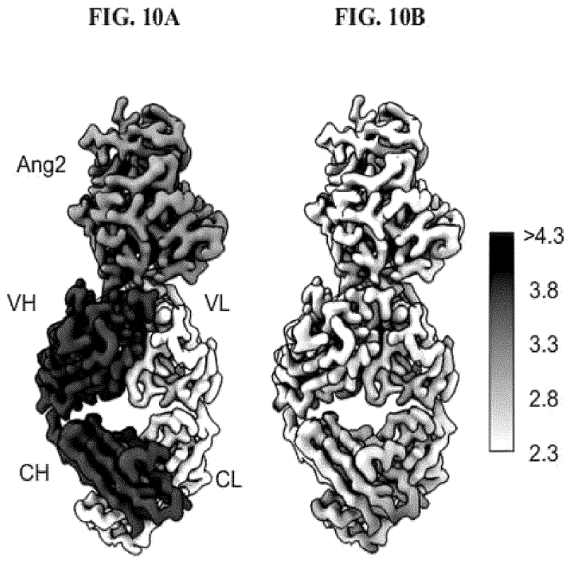

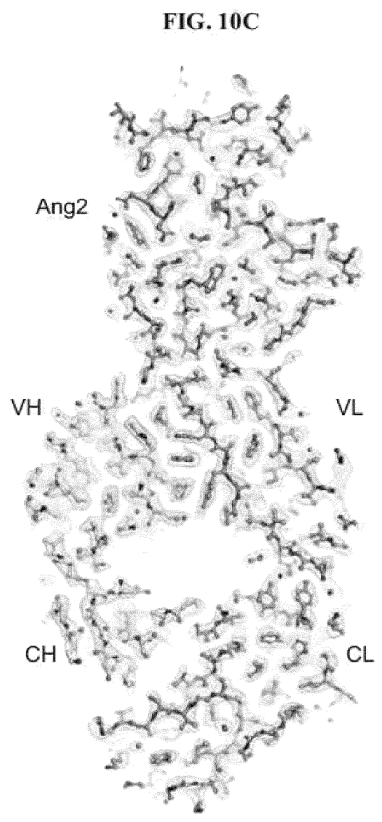

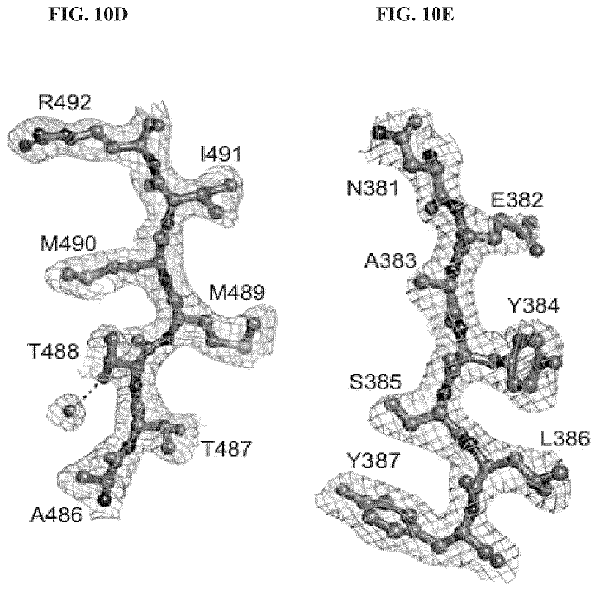

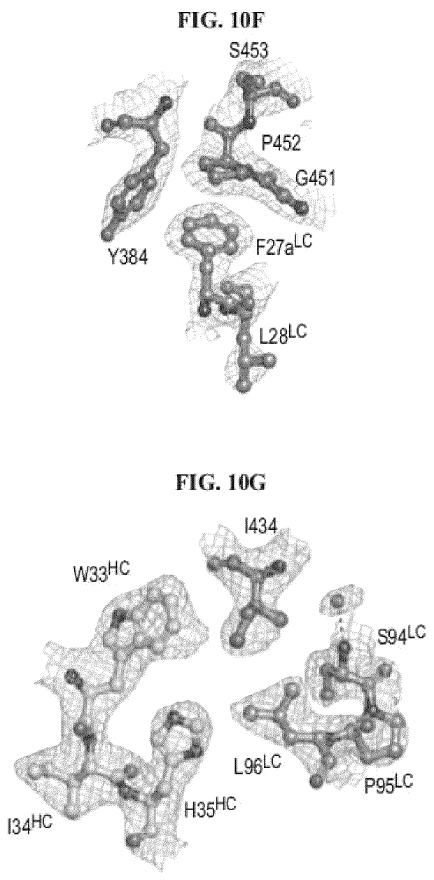

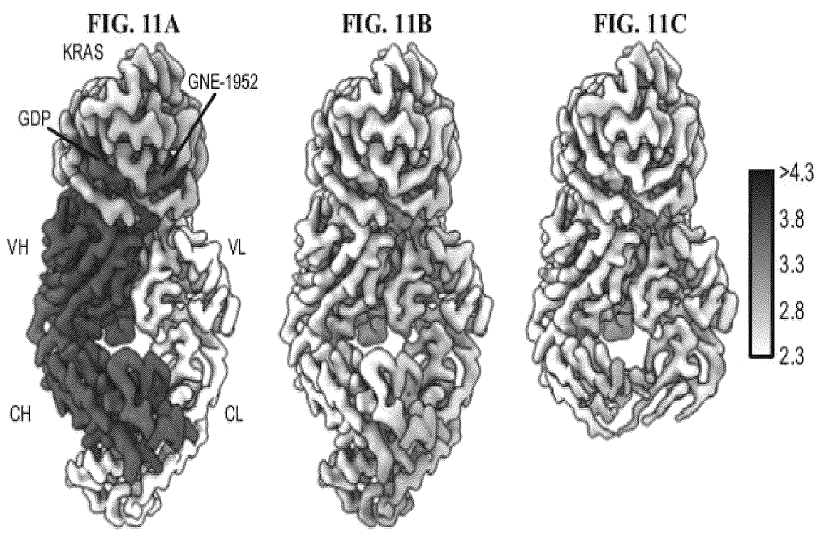

To evaluate if Rigid Fabs could be used to solve a high resolution cryo-EM structure of Ang2, a 6DS variant of the 5 Å12 Fab was generated by introducing cysteine mutations at the same positions as in the E104.v1.6DS Fab and a 74 kDa Ang2-5 Å12.6DS Fab complex was assembled. To further increase the mass of the sample and improve the likelihood of obtaining a high resolution structure, a fusion of the Protein A D domain to Protein G (ProA-ProG) was generated, which formed a ternary complex with 5 Å12.6DS bound to Ang2 (FIG. 9A). Then, a cryo-EM dataset was collected for this complex, but 2D class averages revealed that the vast majority of the particles contained only Ang2 and the 6DS Fab, suggesting that the affinity of ProA-ProG was not high enough to remain bound at the low sample concentrations used during grid freezing (FIG. 9B). Despite the small size of the Ang2-5 Å12.6DS complex, secondary structure in both Ang2 and the Fab were surprisingly clearly resolved in the 2D class averages. Through iterative rounds of heterogeneous refinement, particles containing ProA-ProG were removed, leaving a particle stack containing only the Ang2-5 Å12.6DS complex. These particles were then used to generate a 3D reconstruction with an overall resolution of 2.7 Å, with most regions of Ang2 reaching higher resolutions, up to 2.3 Å (FIG. 10A-B, FIGS. 9C-E). The local resolution is relatively uniform (~2.4-2.8 Å) throughout Ang2 and the Fab, consistent with 5 Å12.6DS being conformationally rigid (FIG. 10B). The overall structure of Ang2 is nearly identical to that observed in the 2.3 Å crystal structure of Ang2 and 5 Å12.WT (RMSD=0.36 Å). The resolution of the map (FIGS. 10C-G) enabled unambiguous placement of side chains in Ang2, the Fab, and the Ang2-Fab interface, and of several water molecules (FIGS. 10C-G), highlighting that using a Rigid Fab design enabled high-resolution structure determination of the 26 kDa Ang2 by cryoEM.

### High-Resolution Cryo-EM Structure of a Small Molecule Bound to 21 kDa KRAS

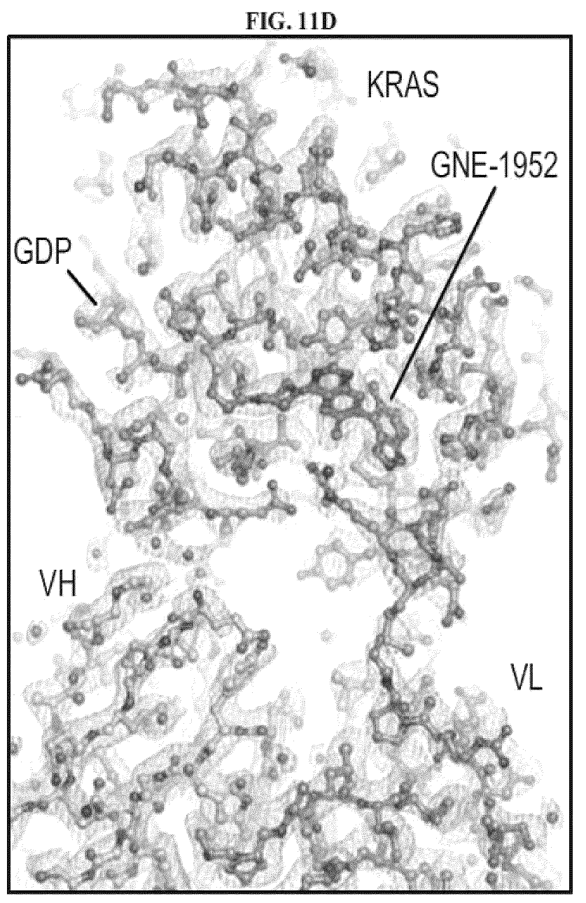

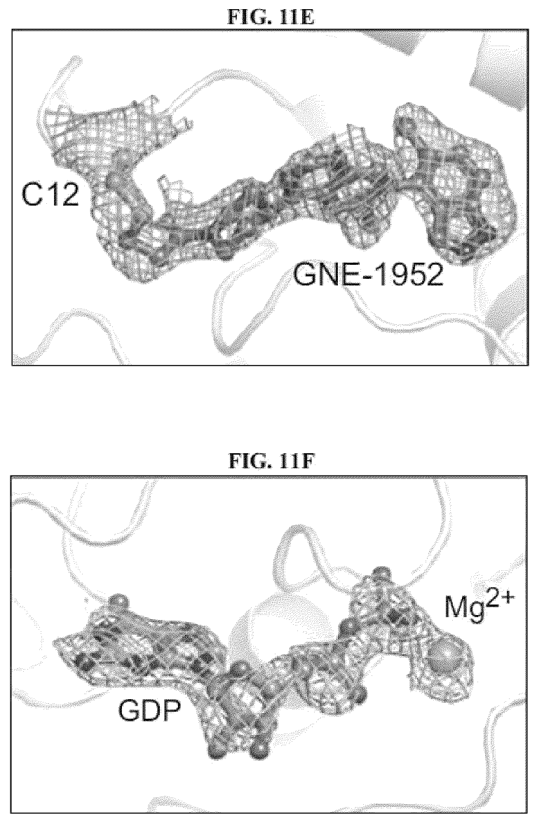

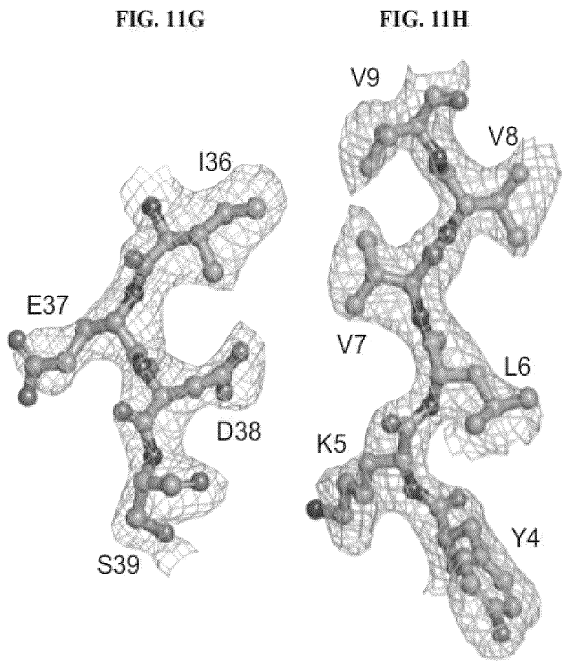

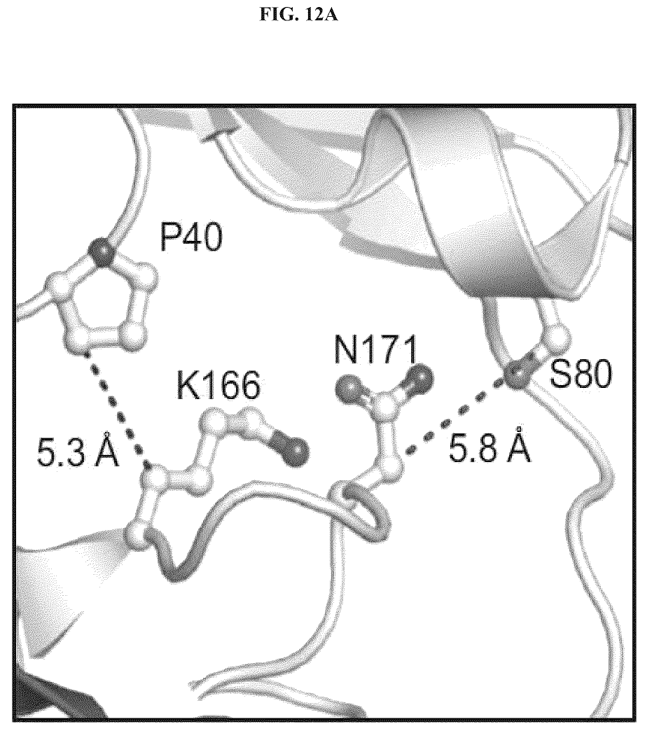

It was reasoned that Rigid Fabs could be used to accelerate structure-based drug design efforts by enabling high-resolution cryo-EM structure determination of small molecule drug targets, many of which are <50 kDa and sometimes can be intractable for structure determination by crystallography or cryo-EM. The 21 kDa GTPase KRAS, a Fab for which has been reported (Davies, Christopher W., et al. “Conformation-locking antibodies for the discovery and characterization of KRAS inhibitors.” Nature biotechnology 40.5 (2022): 769-778.), was chosen. Unlike the Fabs described so far herein, the anti-KRAS Fab 2H11 contains a lambda LC rather than a kappa LC. Lambda LCs are slightly longer, which leads to a wider range of possible elbow angles (Stanfield, Robyn L., et al. “Antibody elbow angles are influenced by their light chain class.” Journal of molecular biology 357.5 (2006): 1566-1574.). Analysis of published crystal structures of 2H11 Fab-KRAS^{G12C} complexes (see, e.g., Davies et al, Nat Biotechnol, 40:769-778, 2022) indicated that this Fab is indeed highly flexible and that the LC cysteine mutations used in the 2DS and 4DS design would likely not be compatible with elbow disulfide formation in this Fab. To form the disulfide corresponding to the LC disulfide introduced in the 2DS design, a cysteine was inserted between N170^{LC} and N171^{LC} rather than generating a N171C^{LC} mutation, as N171^{LC} was likely too far from S80^{LC} for disulfide formation (FIG. 9F). For the second LC disulfide, the mutation K166C^{LC} instead of Q167C^{LC} was used since K166^{LC} was closer to P40^{LC} (FIG. 9F). For the second LC disulfide, the mutation K166C instead of Q167C was used since K166 was closer to P40 (FIG. 9F). Using these mutations, constructs for 4DS and 6DS variants of 2H11 were generated. While the 2H11.6DS Fab did not express, 2H11.4DS was able to be expressed and purified, and LC/MS indicated that all four elbow disulfides had formed. Then, 2H11.4DS was used to form a complex with KRAS^{G12C} conjugated to the covalent inhibitor GNE-1952, and a cryo-EM dataset for this complex was collected (FIG. 9F). This led to a 3D reconstruction with an overall resolution of 2.8 Å for the whole complex, with regions of KRAS and the Fab variable domain having local resolutions of ~2.5 Å (FIGS. 11A-C, FIGS. 9G-L). The local resolution of the Fab constant domain was lower than in the variable domain, unlike 5A12.6DS, consistent with 4DS Fabs being slightly more flexible than 6DS Fabs. Local refinement with a mask to exclude the constant domain led to an overall resolution of 2.7 Å with improvements in local resolution in both KRAS and the Fab variable domain, with several regions reaching 2.3 Å resolution (FIG. 11C). The overall structure of KRAS, GDP, and GNE-1952 in this work is nearly identical to the published crystal structure (RMSD=0.67 Å) (Davies et al, Nat Biotechnol, 40:769-778, 2022). Importantly, the high resolution features at 2.3 Å in the ligand-binding sites for GDP and the inhibitor GNE-1952 enabled unambiguous placement of both ligands, as well as the covalent linkage between C12 and GNE-1952 (FIGS. 11D-F). Sidechains throughout the KRAS^{G12C}portion of the structure were also very well-resolved (FIGS. 11D and 11G-H).

Small proteins (<50 kDa) encompass a vast majority of all known proteins across living organisms, especially in the context of drug targets in humans and pathogens. While several of these proteins are amenable to high-resolution structure determination by x-ray crystallography, many others have had limited success due to low expression, low solubility, or lack of crystallizability. Cryo-EM circumvents these challenges, because it requires low protein amounts and concentrations, yet remains challenging when working with small proteins, which are plagued by low signal-to-noise ratio and lack distinctive features for particle alignment. This study demonstrated that Rigid Fabs increased the effective size of the target protein in a rigid manner and improved particle alignment and pose assignment. This results in high-resolution structure determination of proteins as small as ~21 kDa, revealing features, such as water molecules and unambiguous placement of specific conformations of protein side chains, atoms and small ligands, like inhibitors and co-factors.

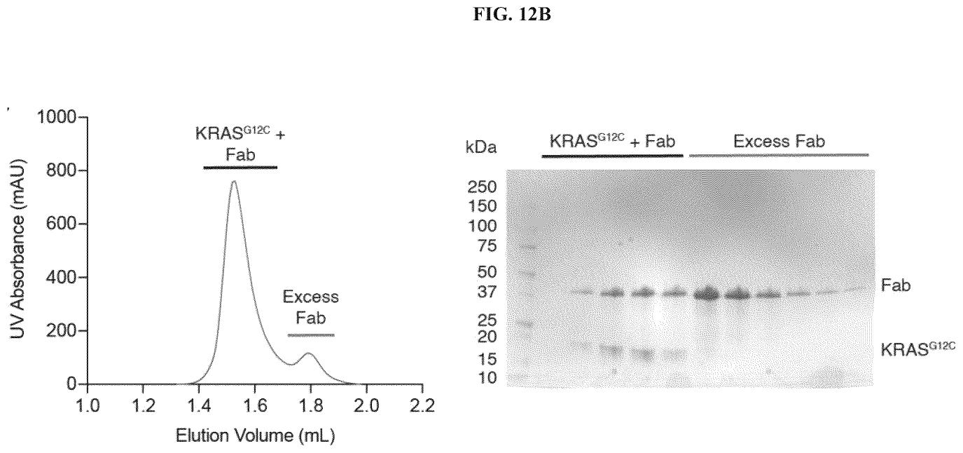

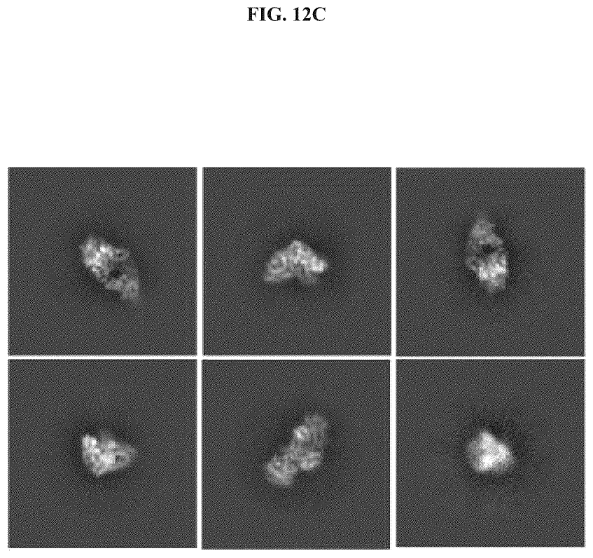

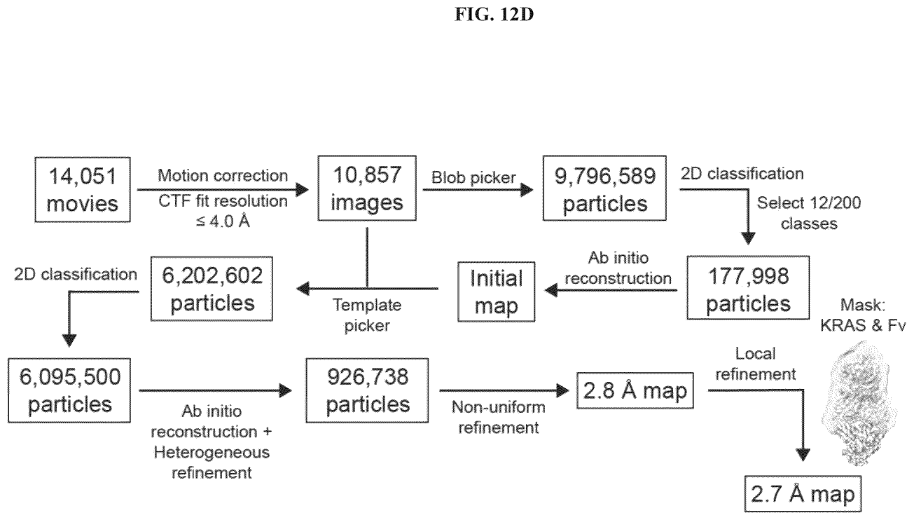

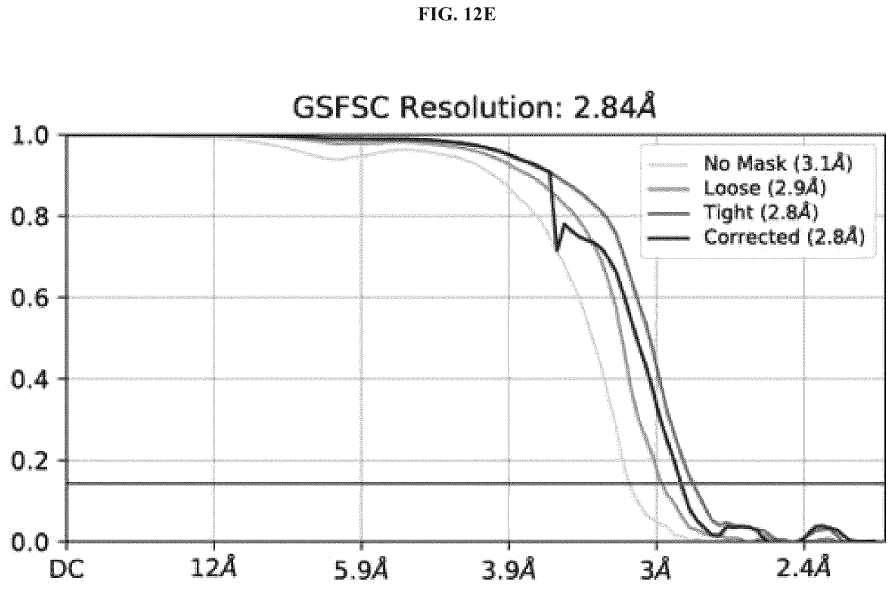

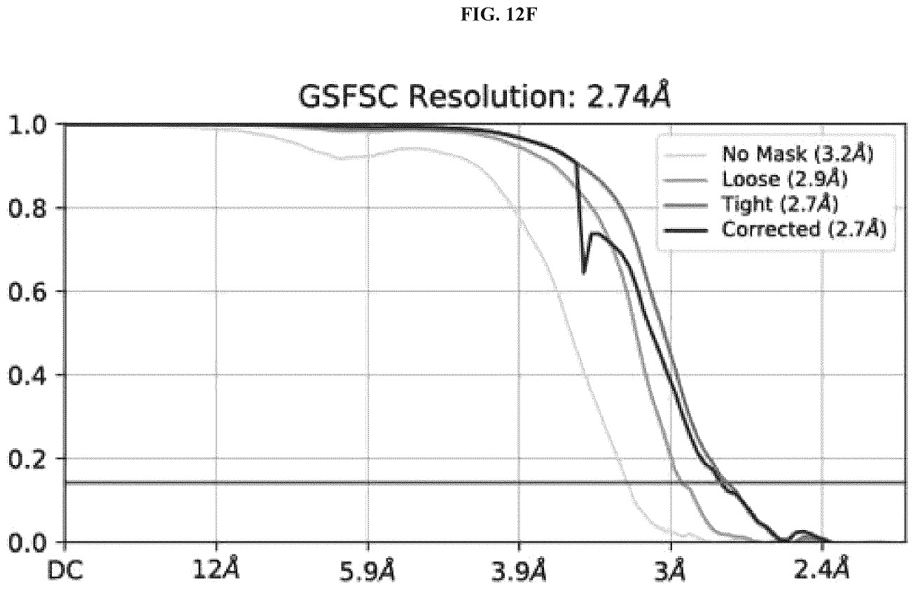

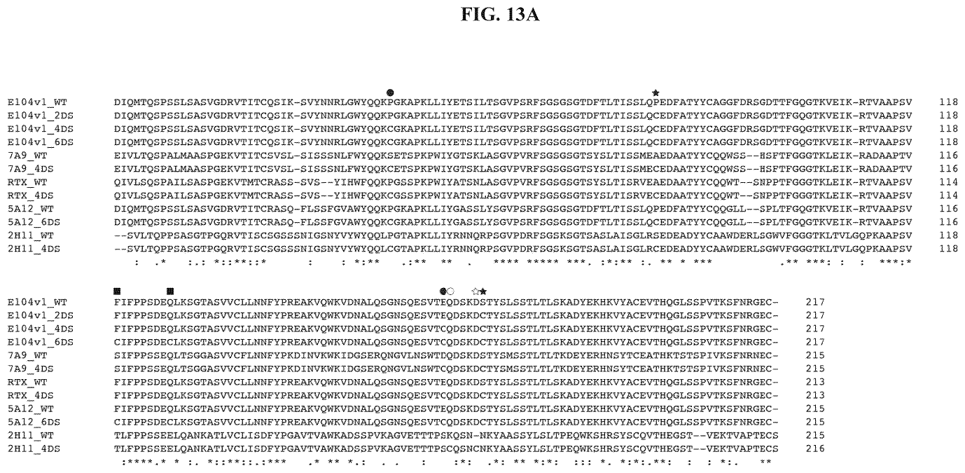

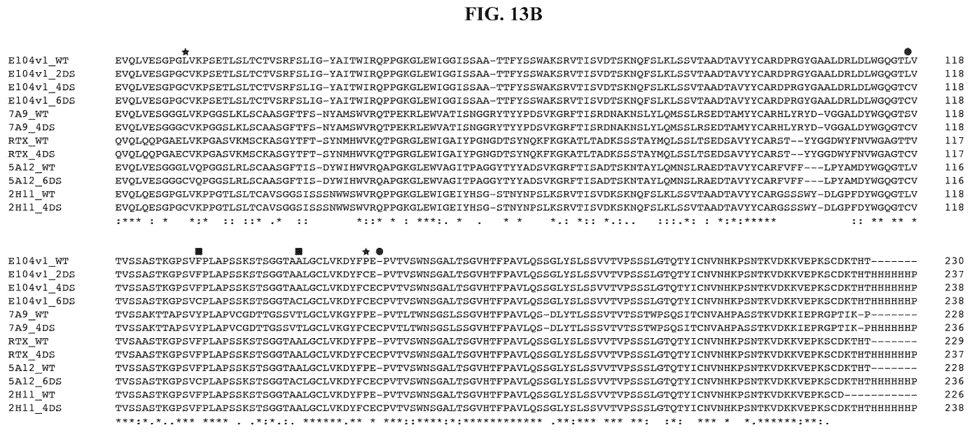

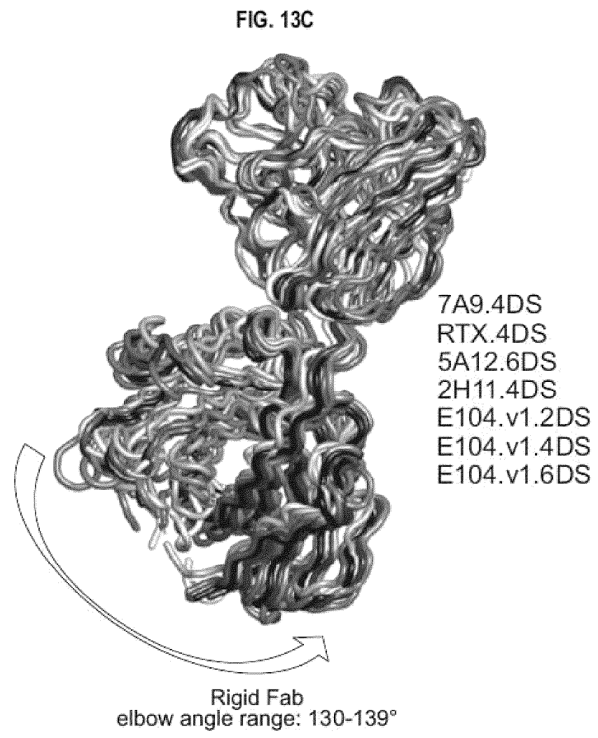

The modularity of our Rigid Fab designs allows their straightforward application to nearly any existing Fab or newly discovered Fab independent of its source and against any target, making this powerful method accessible to a multitude of uses. The results of this study suggest that the introduction of the described four disulfide bonds (4DS) in the Fab elbow region should suffice for high-resolution (~2.5 Å) structure determination in most cases, but that introduction of six disulfides (6DS) can further improve the resolution. The positions for cysteine mutations identified for the 4DS and 6DS variants of the anti-tryptase Fab E104.v1 successfully led to conformationally rigid Fabs for Nav1.7, CD20 and Ang2, all of which contained a Kappa LC like E104.v1 (FIGS. 13A-B). For the KRAS Fab 2H11, which had a lambda LC, the locations of the cysteine mutations in constant domain at the elbow region were altered to accommodate the slight difference in structure compared to the kappa LC (FIG. 12A, FIGS. 13A-B). Fabs exhibit a highly conserved fold and architecture across different species. Sequence alignments with the Rigid Fabs presented in this study revealing locations for cysteine mutations should be sufficient in most cases to identify sites for introducing disulfides to rigidify other Fabs. A pre-existing structure of a fab is not necessary to design a rigid version of the fab. Furthermore, our Rigid Fab designs have resulted in Fabs that adopt a specific conformation (FIG. 13C). The inherent flexibility of Fabs likely makes it possible for most Fabs to sample this conformation, enabling disulfide bond formation using the set of elbow mutations from the designs described herein. Such cases can likewise be identified through sequence alignments with the designs presented in this study, as well as by examining crystal structures of Fabs with high sequence similarity to the Fab of interest. Thus, even in these cases, previous structures of the Fab of interest are not necessary in order to design novel Rigid Fabs.

Rigid Fabs are likely to be most transformative in the study of small (<50 kDa) protein targets. For larger targets (e.g. Nav1.7 tetramer, ~130 kDa, or CD20 dimer, ~45 kDa, ordered molecular weight), which likely feature more intrinsic flexibility than the Fab itself, Rigid Fab technology can also facilitate high-resolution structure determination at the Fab-antigen interface but may need to be coupled with image processing strategies, such as focused refinements or flexibility analysis in order to achieve high resolutions throughout a large, flexible target. Alternatively, or as a complement to such an approach, one may deploy multiple Rigid Fabs against epitopes present on opposite “sides” of the target, in which case, the Fabs would aid not only in particle alignment, but also in 3D classification or flexibility analysis. None of these approaches should be needed when the target proteins are suitably small and rigid, however.

By enabling structure determination of nearly all targets previously intractable by cryo-EM or crystallography, most notably those with molecular weights <50 kDa, Rigid Fab technology can now accelerate basic research into molecular mechanisms of action of proteins involved in pathways of interest, as well as speed up structure-enabled drug discovery and optimization.

### Methods

### Recombinant Protein Expression and Purification

### Wild Type and Rigid Fabs

His-tagged heavy chain and untagged light chain expression constructs were generated by gene synthesis. Fabs were expressed by transient transfection in CHO cells, and the His-tagged Fabs were purified by nickel affinity chromatography followed by size exclusion chromatography (SEC) on a Superdex 200 column equilibrated in 20 mM histidine acetate pH 5.5, 150 mM NaCl. The presence of the engineered disulfides in the Rigid Fab constructs was confirmed by intact protein LC/MS.

### Tryptase

Constructs for human β-tryptase encoding residues I31-P275 with an N-terminal His tag followed by an enterokinase (EK) cleavage site were expressed and purified from Trichoplusia ni insect cells as previously described in Maun et al, J Biol Chem, 298: 9614-9628, 1998. Cultures were harvested 48 hrs post-infection. The supernatant media was filtered through a 0.22 μm filter, and the His-tagged zymogen tryptase was purified by nickel affinity chromatography followed by SEC on a Superdex 200 column equilibrated in SEC buffer (10 mM MOPS pH 6.8, 2 M NaCl). Peak fractions were pooled and concentrated to 2 mg/mL prior to overnight cleavage with 0.1 mg/mL EK (New England Biolabs) at room temperature in 10 mM MOPS pH 6.8, 0.2 M NaCl, 0.5 mg/mL heparin which results in activation and tetramerization of tryptase. Tetrameric tryptase was purified on a Superdex 200 column equilibrated in SEC buffer.

Biotinylated tryptase was generated using a construct with a C-terminal Avi tag and coexpression with BirA to enable in vivo biotinylation. The biotinylated tryptase zymogen was purified via nickel affinity chromatography followed by SEC as described above. Addition of a single biotin was confirmed by intact protein LC/MS.

### Nav1.7

Details of construct design for insect cell expression of Flag-tagged chimeric Nav1.7-NavAb were described previously in Ahuja et al, Science, 350: aac5464, 2015. Expression was performed in T. ni insect cells for 48 hrs. Two liters of insect cell paste were resuspended in 60 mL Cytobuster® supplemented with 1 μg/mL benzonase and 1× protease inhibitor. The mixture was incubated at 22 °C for 5 minutes and transferred to 50 mL conical tubes. 1% glycol-diosgenin (GDN; w/v) was added to solubilize samples. Samples were incubated with anti-Flag magnetic beads at 4 °C for 2 hours with rotary mixing. To wash unbound proteins from the magnetic beads, a magnetic rack was used Beads were washed 4× with 10 CV Wash Buffer containing 0.042% GDN. Proteins were eluted with Elution Buffer containing 0.042% GDN and 150 ug/mL Flag peptide. Fractions were pooled and separated on a Superose 6 10/300 column equilibrated in Gel Filtration Buffer (10 mM Tris pH 8.0, 100 mM NaCl, 0.042% GDN).

### CD20

Details of construct design for insect cell expression of His-tagged CD20 were described previously in Rouge et al, Science, 367: 1224-1230, 2020. Expression was performed in T. ni insect cells for 48 hours. Two liters of insect cell paste was resuspended in 60 mL cell resuspension buffer (25 mM Tris pH 7.5, 300 mM NaCl, 10% glycerol) supplemented with 1 μg/mL benzonase and 1× protease inhibitor. The mixture was incubated at 22 °C for 5 minutes and transferred to 50 mL conical tubes.

1% GDN/0.1% cholesteryl hemisuccinate (CHS; w/v) was added to solubilize samples, and as well as washed nickel magnetic beads. Samples were incubated with Ni-charged magnetic beads at 4 °C for 2 hours with rotary mixing. To wash unbound proteins from the magnetic beads, a magnetic rack was used. Beads were washed 4× with 10 CV Wash Buffer containing 0.02% GDN/0.002% CHS. Proteins were eluted with elution buffer containing 300 mM imidazole. Fractions were pooled and separated on a Superose 6 10/300 column equilibrated in gel filtration buffer (25 mM Tris pH 7.5, 150 mM NaCl, 0.02% GDN/0.002% CHS).

### Ang2

C-terminally His-tagged Ang2 (residues E277-F496) was expressed and purified from T. ni insect cells as described previously in Koenig et al, J Biol Chem, 290: 21773-21786, 2015. Cultures were harvested 48 hours post-infection. The supernatant media was filtered through a 0.22 μm filter, and the protein was purified by nickel affinity chromatography followed by SEC on a Superdex 200 column equilibrated in 20 mM Tris pH 7.5, 150 mM NaCl.

### ProA-ProG

To generate an N-terminally His-tagged construct for bacterial expression of a Protein A-Protein G (ProA-ProG) fusion protein, residues F100-K153 from Protein A and residues T368-G430 from Protein G were linked with a 3×GS linker. The ProA-ProG fusion protein was expressed in BL21(DE3) cells in TB autoinduction media for 48 hours at 17 °C. Cell pellets were resuspended in lysis buffer (50 mM Tris pH 8.0, 500 mM NaCl, 10% glycerol) supplemented with 20 mM imidazole, 1 EDTA-free protease inhibitor tablet, 1 μg/mL benzonase, lysis detergents (0.3% Sb3-14 and 0.03% C7BzO), and 20 mg lysozyme. The solution was homogenized and incubated with 2 ml per 1 L pellet of Ni-charged MagBeads for 30 minutes at room temperature. To wash unbound proteins from the magnetic beads, a magnetic rack was used. Beads were washed 4× with 10 CV lysis buffer supplemented with 20 mM imidazole. The bound protein was eluted with lysis buffer supplemented with 300 mM imidazole. The protein was then concentrated and purified via SEC on a Superdex 75 column equilibrated in 20 mM Tris pH 7.5, 150 mM NaCl.

### KRAS^{G12C}

A “Cys-light” construct of KRAS^{G12C} (residues M1-K169) was generated for E. coli expression by mutating all Cys residues except C12 to Ser. The protein was expressed in BL21(DE3) cells induced with 0.5 mM IPTG overnight at 16 °C. Cells were then harvested, resuspended in lysis buffer (50 mM HEPES pH 7.0, 300 mM NaCl, 5% glycerol, 5 mM MgCl₂, 10 uM GDP, 1 mM TCEP) supplemented with 1 mM PMSF, 1 μg/mL benzonase, and 1× protease inhibitor, and lysed using a microfluidizer. The clarified supernatant was passed over a NiNTA agarose column, and the protein was eluted with lysis buffer supplemented with 300 mM imidazole. The eluted protein was dialyzed into dialysis buffer (50 mM HEPES pH 7.0, 300 mM NaCl, 5 mM MgCl₂, 10% glycerol, 1 mM TCEP, 10 μM GDP) and incubated overnight with TEV protease to cleave the His tag. The sample was passed again over a Ni-NTA column to remove uncleaved protein. The flowthrough was concentrated and purified via SEC on a Superdex 75 column equilibrated in SEC buffer (50 mM HEPES pH 7.0, 100 mM NaCl, 1 mM MgCl₂, 1 mM TCEP, 10 μM GDP).

### Biolayer Interferometry

All binding assays were performed in 20 mM Tris pH 7.5, 150 mM NaCl, 0.1% BSA, 0.01% Tween20. Biotinylated tryptase was captured on streptavidin SA biosensors. Assays were performed in triplicate on an OctetRED384. Sensorgrams were normalized to a reference well containing only buffer. Equilibrium binding constants were determined by plotting the average response values versus Fab concentration and fitting to a global one site-specific binding model in Prism.

### Protein Crystallization, Data Collection and Processing

E104.v1.4DS S112F was crystallized at a concentration of 8 mg/mL via vapor diffusion in sitting well drops at 19 °C in 0.2 M Na citrate and 20% PEG 3350. Crystals were cryoprotected in mother liquor supplemented with 10% glycerol. Diffraction data were collected at the Advanced Light Source (ALS) beamline 5.0.2. Data were processed to a resolution of 2.0 Å in XDS, and phases were obtained through molecular replacement with Phaser, using E104v1.WT variable and constant domains from a previously published crystal structure of the E104v1.WT-Tryptase complex (PDB: 6VVU, chains G and I) as search models.

E104.v1.4DS A114F was crystallized at a concentration of 10 mg/mL via vapor diffusion in sitting well drops at 19 °C in 0.1 M Na citrate pH 4.5 and 20% PEG 4000. Crystals were cryoprotected in mother liquor supplemented with 10% glycerol. Diffraction data were collected at the Advanced Light Source (ALS) beamline 5.0.2. Data were processed to a resolution of 2.01 Å in XDS, and phases were obtained through molecular replacement with Phaser, using the crystal structure of E104v1.4DS S112F as the search model.

E104.v1.5DS and E104v1.6DS were crystallized at concentrations of 10 mg/mL and 7 mg/mL, respectively via vapor diffusion in hanging well drops at 19 °C in 0.1 M Na Citrate pH 4.5 and 26% PEG 4000. Crystals were cryoprotected in mother liquor supplemented with 20% glycerol. Diffraction data were collected at Stanford Synchrotron Radiation Lightsource (SSRL) beamline 1.2.1. Data were processed to a resolution of 2.14 Å for E104.v1.5DS and 2.71 Å for E104.v1.6DS in XDS, and phases were obtained through molecular replacement with Phaser, using the crystal structure of E104v1.4DS A114F as the search model.

Iterative rounds of model building and refinement were performed in COOT and Phenix. (See, Emsley et al, Acta Crystallogr Sect D, 66: 486-501, 2010 and Liebschner et al, Acta Crystallogr. Sect. D., 75: 861-877, 2019). Figures were generated using PyMOL.

Data collection and refinement statistics for Rigid Fab crystal structures are shown in Table 2. E104.v1.4DS.S112F is an anti-tryptase Fab E104.v1 with the 4DS disulfide bridge design as described herein, which has an additional S112F mutation. E104.v1.4DS.A114F is an anti-tryptase Fab E104.v1 with the 4DS disulfide bridge design as described herein, which has an additional A114F mutation. E104.v1.5DS is an anti-tryptase Fab E104.v1 with the 5DS disulfide bridge design as described herein. E104.v1.6DS is an anti-tryptase Fab E104.v1 with the 6DS disulfide bridge design as described herein.

TABLE 2
Data collection and refinement statistics for Rigid Fab crystal structures.

| | E104.v1.4DS.S112F | | E104.v1.4DS.A114F | | E104.v1.5DS | | E104.v1.6DS | |
|---|---|---|---|---|---|---|---|---|
| PDB ID | 8VEG | | 8VGE | | 8VGF | | 8VGG | |
| Data | | | | | | | | |
| collection | | | | | | | | |
| Space group | P 61 2 2 | | P 61 2 2 | | P 61 2 2 | | P 61 2 2 | |
| Cell | | | | | | | | |
| dimensions | | | | | | | | |
| a, b, c (Å) | 116.34, 116.34, 136.05 | | 115.10, 115.10, 137.83 | | 115.52, 115.52, 136.42 | | 115.49, 115.49, 136.04 | |
| α, β, γ (°) | 90, 90, 120 | | 90, 90, 120 | | 90, 90, 120 | | 90, 90, 120 | |
| Resolution | 38.08-2.0 | | 46.869-2.010 | | 36.44-2.14 | | 36.42-2.71 | |
| (Å) | (2.072-2.0) | | (2.082-2.010) | | (2.216-2.14) | | (2.807-2.710) | |
| R_{sym} | 0.07047 | (1.623) | 0.08241 | (1.578) | 0.1421 | (1.208) | 0.2333 | (1.693) |
| I/σI | 27.52 | (2.29) | 26.57 | (2.27) | 15.14 | (4.24) | 15.92 | (2.42) |
| CC½ (%) | 1 | (0.854) | 1 | (0.808) | 0.998 | (0.891) | 0.997 | (0.799) |
| Completeness | 99.05 | (99.95) | 98.58 | (99.64) | 99.93 | (100.00) | 99.85 | (100.00) |
| (%) | | | | | | | | |
| Redundancy | 19.3 | (19.6) | 18.8 | (19.5) | 15.6 | (14.6) | 19.6 | (19.7) |
| Refinement | | | | | | | | |
| Resolution | 2.0 | | 2.01 | | 2.14 | | 2.71 | |
| (Å) | | | | | | | | |
| No. | 717900 | (71762) | 686885 | (69671) | 471925 | (43369) | 296265 | (28711) |

| | | | | |
|---|---|---|---|---|
| reflections | | | | |
| R_{work}/R_{free} | 0.2147/0.2434 | 0.2037/0.2321 | 0.1791/0.2189 | 0.2066/0.2579 |
| No. atoms | 3428 | 3487 | 3468 | 3172 |
| Protein | 3228 | 3279 | 3225 | 3161 |
| Ligand/ion | N/A | N/A | N/A | N/A |
| Water | 200 | 208 | 243 | 11 |
| B-factors | 53.83 | 54.70 | 55.74 | 64.94 |
| Protein | 53.90 | 54.88 | 55.81 | 65.02 |
| Ligand/ion | N/A | N/A | N/A | N/A |
| Water | 52.70 | 51.85 | 54.89 | 41.51 |
| R.m.s. | | | | |
| deviations | | | | |
| Bond | 0.006 | 0.004 | 0.011 | 0.002 |
| length (Å) | | | | |
| Bond | 1.25 | 0.69 | 1.28 | 0.60 |
| angles (°) | | | | |

### Cryo-EM Sample Preparation, Data Acquisition, and Data Processing

### Tryptase

Tryptase-Fab complexes were prepared by incubating tetrameric tryptase with a 2-fold molar excess of Fab on ice for 30 min. The tryptase-Fab complex was then separated via size exclusion chromatography on a Superdex 200 3.2/300 or Superose 6 3.2/300 column equilibrated in 20 mM MOPS pH 5.5, 800 mM NaCl. The peak fraction was subjected to mild crosslinking with 2.5 mM BS3 at room temperature for 10 min. The crosslinking reaction was quenched by addition of 100 mM Tris pH 7.5. Four μL of this sample were then applied to a grid, blotted in a Vitrobot MarkIV at 4 °C and 100% humidity, using a blotting time of 3 s and a blot force of 7, and plunge-frozen in liquid ethane cooled by liquid nitrogen. The tryptase-E104.v1.WT Fab complex was applied to a glow-discharged Quantifoil R0.6/1 Cu400 holey carbon grid. The tryptase-E104.v1.2DS complex was applied to a glow-discharged Quantifoil R0.6/1 Au300 holey carbon grid. The tryptase-E104.v1.4DS and tryptase-E104.v1.6DS complexes were applied to Quantifoil R0.6/1 Au300 holey carbon grids treated overnight with a thiol-reactive, self-assembling reaction mixture of 4 mM monothiolalkane(C11)PEG6-OH (11-mercaptoundecyl) hexaethyleneglycol (SPT-0011P6, SensoPath Technologies Inc., Bozeman, MT). (See, Meyerson et al, Sci Rep-uk, 4:7084, 2014.) Before application of the protein, the grids were removed from the SAM solution and rinsed with ethanol.

Movie stacks for tryptase-E104.v1.WT Fab were collected using SerialEM (Mastronarde et al, J Struct Biol, 152:36-51, 2005) on a Titan Krios operated at 300 kV and equipped with a BioQuantum energy filter operated with a 20 eV energy slit with a K2 Summit direct electron detector camera. Images were recorded at a nominal magnification of 165,000×, corresponding to 0.824 Aper pixel. Each image stack contains 50 frames recorded every 0.2 s giving an accumulated dose of 54 e/Å² and a total exposure time of 10 s. Images were recorded with a set defocus range of 0.5 to 1.5 μm.

Movie stacks for tryptase-E104.v1.2DS, tryptase-E104.v1.4DS, and tryptase-E104.v1.6DS were collected using SerialEM on a Titan Krios operated at 300 kV and equipped with a BioQuantum energy filter operated with a 20 eV energy slit with a K3 Summit direct electron detector camera. Images were recorded in EFTEM mode at a magnification of 105,000× corresponding to 0.838 Å per pixel, using a 20 eV energy slit. Each image stack contains 60 frames recorded every 0.05 s for an accumulated dose of ~65 e/Å² and a total exposure time of 3 s. Images were recorded with a set defocus range of 0.5 to 1.5 μm.

For all four tryptase-Fab complexes, image processing was performed as described in FIG. 5. Motion correction, CTF estimation, and particle picking were performed in cisTEM (Grant et al, eLife, 7: e35383, 2018). Particles were extracted from micrographs with CTF fit resolution less than 6 Å with a box size of 400 px and imported into Cryosparc. The particles were binned to a box size of 128 px and subjected to 2D classification to remove junk particles. The remaining particles were subjected to iterative rounds of multi-class ab initio reconstruction and heterogeneous refinement. The quality of the particles in the best classes was evaluated by running non-uniform refinement. The unbinned particles in the best class were then exported back into cisTEM. Prior to export, the Particle Sets tool in Cryosparc was used to randomly select a subset of the best particles from the 2DS, 4DS, and 6DS datasets in order to match the final number of particles in the WT dataset. In cisTEM, 3D refinement was performed using Auto-refine, using a low-pass filtered map from non-uniform refinement in Cryosparc as a reference. The auto-refined maps were then subjected to CTF refinement and manual refinement. The highest resolution used during refinement is indicated in FIGS. 5D, 5H, 5L, and 5P. Local resolution maps were calculated using Relion (Scheres et al, J Struct Biol, 180:519-530, 2012 Cryo-EM data collection, refinement, and validation statistics for Tryptase-E104.v1 complexes are shown in Table 3.

The crystal structure of the tryptase-E104.v1 WT complex (PDB: 6VVU) was used as an initial model to dock into the cryo-EM maps in ChimeraX. The resulting models were rebuilt and refined using COOT, ISOLDE, and Phenix. See, Maun et al, Nat Commun, 11:6435, 2020, Pettersen et al, Protein Sci, 30: 70-82, 2020, and Croll et al, Acta Crystallogr Sect D: Struct Biol, 74:519-530, 2018. Sharpened maps were generated using the Cryo-EM module in COOT. Figures were generated using ChimeraX and PyMOL.

TABLE 3
Cryo-EM data collection, refinement, and validation
statistics for Tryptase-E104.v1 complexes.

| | Tryptase- | Tryptase- | Tryptase- | Tryptase- |
|---|---|---|---|---|
| | E104.v1.WT | E104.v1.2DS | E104.v1.4DS | E104.v1.6DS |
| EMDB ID | EMD-43200 | EMD-43201 | EMD-43202 | EMD-43203 |
| PDB ID | 8VGH | 8VGI | 8VGJ | 8VGK |
| Data collection and | | | | |
| processing | | | | |
| Magnification | 165,000 | 105,000 | 105000 | 105000 |
| Voltage (kV) | 300 | 300 | 300 | 300 |
| Detector | K2 | K3 | K3 | K3 |
| Electron exposure | 54.5 | 64.0 | 66.6 | 64.0 |
| (e⁻/Å) | | | | |
| Defocus range (μm) | 0.6-1.6 | 0.5-1.5 | 0.5-1.5 | 0.5-1.5 |
| Pixel size | 0.824 | 0.838 | 0.838 | 0.838 |
| (micrographs) (Å) | | | | |
| Symmetry imposed | D2 | D2 | D2 | D2 |
| Initial particle | 841,718 | 852,218 | 617,263 | 1,140,931 |
| images (no.) | | | | |
| Final particle images | 253,508 | 253,508 | 253,508 | 253,508 |
| (no.) | | | | |
| Pixel size (map) (Å) | 1.0 | 1.0 | 1.0 | 1.0 |
| Map resolution (Å) | 2.9 | 2.7 | 2.5 | 2.4 |
| FSC threshold | 0.143 | 0.143 | 0.143 | 0.143 |
| Map resolution | 2.9-18.8 | 2.7-21.4 | 2.5-19.4 | 2.4-19.7 |
| range (Å) | | | | |
| Refinement | | | | |
| Initial model used | 6VVU | 6VVU | 6VVU | 6VVU |
| (PDB code) | | | | |
| Model resolution | 2.9 | 2.7 | 2.5 | 2.5 |
| (Å) | | | | |
| FSC threshold | 0.5 | 0.5 | 0.5 | 0.5 |
| Model resolution | 2.9-18.8 | 2.7-21.4 | 2.5-19.4 | 2.5-19.7 |
| range (Å) | | | | |
| Map sharpening B | −100 | −100 | −100 | −100 |
| factor (Å²) | | | | |
| Model composition | | | | |
| Non-hydrogen | 20472 | 20480 | 20579 | 20567 |
| atoms | | | | |
| Protein residues | 2668 | 2668 | 2672 | 2672 |
| Ligands | 0 | 0 | 0 | 0 |
| R.m.s. deviations | | | | |
| Bond lengths (Å) | 0.011 | 0.010 | 0.007 | 0.003 |
| Bond angles (°) | 0.944 | 0.884 | 0.826 | 0.768 |
| Validation | | | | |
| Molprobity score | 1.82 | 1.63 | 1.80 | 1.51 |
| Clashscore | 6.37 | 3.83 | 4.25 | 2.53 |
| Poor rotamers | 0.13 | 0.70 | 1.57 | 0.30 |
| (%) | | | | |
| Ramachandran plot | | | | |
| Favored (%) | 92.41 | 92.75 | 93.11 | 92.27 |
| Allowed (%) | 7.02 | 6.98 | 6.59 | 7.54 |
| Disallowed (%) | 0.57 | 0.27 | 0.30 | 0.19 |
| Rama-Z score | | | | |
| Whole | −1.29 | −0.92 | −1.28 | −0.96 |
| Helix | −3.23 | −2.78 | −2.56 | −2.32 |
| Sheet | −0.13 | 0.10 | 0.22 | 0.54 |
| Loop | −1.01 | −0.86 | −1.39 | −1.26 |

### Nav1.7

Nav1.7-7A9 complexes were prepared by incubating Nav1.7 with a 1.2× molar excess of Fab at 4 °C for 30 minutes and separated on a Superose® 6 3.2/300 column. Three μL from the peak fraction were applied to R2/2 Au300 holey carbon grids treated with a thiol-reactive, self-assembling reaction mixture of 4 mM monothiolalkane(C11)PEG6-OH (11-mercaptoundecyl) hexaethyleneglycol. Before application of the protein, the grids were removed from the SAM solution and rinsed with ethanol. The grids were blotted in a Vitrobot MarkIV at 4 °C and 100% humidity, using a blotting time of 3 s and a blot force of 7, and plunge-frozen in liquid ethane cooled by liquid nitrogen.

Movie stacks were collected using EPU on a Titan Krios operated at 300 kV and equipped with a Selectris and a Falcon4 detector. Images were recorded at a magnification of 165,000× corresponding to 0.731 Å per pixel, using a 20 eV energy slit. Each image stack contains 1077 frames recorded every 0.005 s for an accumulated dose of ~44 e/Å² and a total exposure time of 5 s. Images were recorded with a set defocus range of 0.5 to 1.5 μm.

All image processing was performed in CryoSPARC, as summarized in FIG. 7. Patch motion correction, patch CTF estimation, and particle picking were performed using CryoSPARC Live. Micrographs with CTF fit resolutions worse than 6.5 Å were rejected. The blob picker was used with a minimum radius of 160 Å and maximum radius of 220 Å. Particles were extracted with a box size of 512 px and binned to 128 px. 2D classification on a small subset of the data was used to identify 2D classes for generation of initial model via ab initio reconstruction. This model was used to generate templates for particle picking with a radius of 220 Å. The resulting particle stack was subjected to 2D classification to remove junk particles. The remaining particles were subjected to iterative rounds of multi-class ab initio reconstruction and heterogeneous refinement. The quality of the particles in the best classes were evaluated by running non-uniform refinement. The particles from the best classes were subjected to 3D classification into 10 classes with a mask around Nav1.7 and the Fab variable domain. Classes where the 4-helix bundle was best-resolved were selected and re-extracted with a box size of 512 px and binned to 400 px. Non-uniform refinement of the re-extracted particles led to a consensus 3D reconstruction. Local refinement with a mask around both Fabs was used to improve alignments of the Fabs prior to particle subtraction to remove the Fabs. The signal-subtracted particles were then subjected to local refinement with a mask around Nav1.7. Phenix.combine_focused_maps was used to generate a composite map using the consensus map and local refinement maps focused on the Fabs and Nav1.7 as inputs. The local resolution of the composite map was calculated using the Local Resolution job in Phenix with the composite half maps produced by Phenix.combine_focused_maps.

Previously published structures of the Nav1.7-ProTx2-7A9.WT complex (PDB: 6N4Q) and Nav1.7-NavAb (PDB: 5EK0) (Ahuja et al, Science, 350:aac5464, 2015) were used as an initial models for the 7A9 Fab and Nav1.7, respectively, to dock into the cryo-EM maps in ChimeraX. The resulting models were rebuilt and refined using COOT, ISOLDE, and Phenix. Sharpened maps were generated using the Cryo-EM module in COOT. Figures were generated using ChimeraX and PyMOL.

### CD20

CD20-RTX complexes were prepared by incubating CD20 with a 1.2 molar excess of Fab at 4 °C for 30 minutes and separated on a Superose 6 3.2/300 column. 3 μL from the peak fraction were applied to Quantifoil R0.6/1 Au300 holey carbon grids treated overnight with a thiol-reactive, self-assembling reaction mixture of 4 mM monothiolalkane(C11)PEG6-OH (11-mercaptoundecyl) hexaethyleneglycol. Before application of the protein, the grids were removed from the SAM solution and rinsed with ethanol. The grids were blotted in a Vitrobot MarkIV at 4 °C and 100% humidity, using a blotting time of 5 s and a blot force of 8, and plunge-frozen in liquid ethane cooled by liquid nitrogen.

Movie stacks for CD20-RTX.WT were collected from 1 grid using EPU on a Titan Krios operated at 300 kV and equipped with a Selectris and a Falcon4 detector. Images were recorded at a magnification of 165,000× corresponding to 0.731 Å per pixel, using a 20 eV energy slit. Each image stack contains 1011 frames recorded every 0.004 s for an accumulated dose of 37 e/Å² and a total exposure time of 4 s. Images were recorded with a set defocus range of 0.5 to 1.5 μm.

Movie stacks for CD20-RTX.4DS were collected from 4 grids using EPU on a Titan Krios operated at 300 kV and equipped with a Selectris energy filter and a Falcon4 detector. Images were recorded at a magnification of 165,000× corresponding to 0.731 Å per pixel, using a 20 eV energy slit. Each image stack contains 1001 frames recorded every 0.005 s for an accumulated dose of 45 e/Å² and a total exposure time of 5 s. Images were recorded with a set defocus range of 0.5 to 1.5 μm.

All image processing was performed in CryoSPARC, as summarized in FIG. 8. Patch motion correction, patch CTF estimation, and particle picking were performed using CryoSPARC Live. Micrographs with CTF fit resolutions worse than 4.0 Å were rejected. The blob picker was used with a minimum radius of 150 Å and maximum radius of 220 Å. Particles were extracted with a box size of 512 px and binned to 128 px. 2D classification on a small subset of the data was used to identify 2D classes for generation of initial model via ab initio reconstruction. This model was used to generate templates for particle picking with a radius of 200 Å. The resulting particle stack was subjected to 2D classification to remove junk particles. The remaining particles were subjected to iterative rounds of multi-class ab initio reconstruction and heterogeneous refinement. The quality of the particles in the best classes were evaluated by running non-uniform refinement. These particles from the best classes were re-extracted with a box size of 512 px and binned to 400 px. Non-uniform refinement of the re-extracted particles led to a consensus 3D reconstruction. Local refinement was performed with a mask around the variable domains of both Fabs and CD20. The fulcrum for the local refinement was also shifted to the center of mass for the CD20 portion of the structure. The resulting volume was used as input for another local refinement with a mask around only CD20. Phenix.combine_focused_maps was used to generate a composite map using the consensus map and local refinement maps as inputs. The local resolution of the composite map was calculated using the Local Resolution job in Phenix with the composite half maps produced by Phenix.combine_focused_maps.

The model for the structure of the CD20-RTX.WT complex (PDB: 6VJA) (Rougé et al, Science, 367: 1224-1230, 2020) was used as an initial model to dock into the cryo-EM maps in ChimeraX. The resulting models were rebuilt and refined using COOT, ISOLDE, and Phenix. Sharpened maps were generated using the Cryo-EM module in COOT. Figures were generated using ChimeraX and PyMOL.

Cryo-EM data collection, refinement, and validation statistics for Nav1.7-7A9 and CD20-RTX complexes are shown in Table 4.

TABLE 4
Cryo-EM data collection, refinement, and validation
statistics for Nav1.7-7A9 and CD20-RTX complexes.

| | Nav1.7- | Nav1.7- | CD20- | CD20- |
|---|---|---|---|---|
| | 7A9.WT | 7A9.4DS | RTX.WT | RTX.4DS |
| EMDB ID | EMD-43204 | EMD-43208 | EMD-43212 | EMD-43216 |
| | (composite | (composite | (composite | (composite |
| | map), | map), | map), | map), |
| | EMD-43205 | EMD-43209 | EMD-43213 | EMD-43217 |
| | (consensus | (consensus | (consensus | (consensus |
| | map), | map), | map), | map), |
| | EMD-43206 | EMD-43210 | EMD-43214 | EMD-43218 |
| | (focused | (focused | (focused | (focused |
| | refinement of | refinement of | refinement of | refinement of |
| | 7A9 Fab), and | 7A9 Fab), and | Fab variable | Fab variable |
| | EMD-43207 | EMD-43211 | domain and | domain and |
| | (focused | (focused | CD20), and | CD20), and |
| | refinement of | refinement of | EMD-43215 | EMD-43219 |
| | Nav1.7) | Nav1.7) | (focused | (focused |
| | | | refinement of | refinement of |
| | | | CD20) | CD20) |
| PDB ID | 8VGL | 8VGM | 8VGN | 8VGO |
| | (model) | (model) | (model) | (model) |
| Data collection | | | | |
| and processing | | | | |
| Magnification | 165,000 | 165,000 | 165,000 | 165,000 |
| Voltage (kV) | 300 | 300 | 300 | 300 |
| Detector | Falcon 4 | Falcon 4 | Falcon 4 | Falcon 4 |
| Electron | 44.0 | 44.0 | 44.0 | 44.0 |
| exposure (e⁻/Å) | | | | |
| Defocus range | 0.8-1.8 | 0.8-1.8 | 0.8-1.8 | 0.8-1.8 |
| (μm) | | | | |
| Pixel size | 0.731 | 0.731 | 0.731 | 0.731 |
| (micrographs) | | | | |
| (Å) | | | | |
| Symmetry | C2 | C2 | C2 | C2 |
| imposed | | | | |
| Initial particle | 1,752,500 | 1,312,570 | 1,262,843 | 1,580,660 |
| images (no.) | | | | |
| Final particle | 391,335 | 362,778 | 390,855 | 375,469 |
| images (no.) | | | | |
| Pixel size (map) | 0.9357 | 0.9357 | 0.9357 | 0.9357 |
| (Å) | | | | |
| Map resolution | 2.6 | 2.6 | 2.5 | 2.6 |
| (Å) | | | | |
| FSC threshold | 0.143 | 0.143 | 0.143 | 0.143 |
| Map resolution | 2.1-33.5 | 2.1-37.6 | 2.1-37.0 | 2.0-40.3 |
| range (Å) | | | | |
| Refinement | | | | |
| Initial model | 6N4Q, 5EK0 | 6N4Q, 5EK0 | 6VJA | 6VJA |
| used (PDB code) | | | | |
| Model resolution | 2.6 | 2.6 | 2.6 | 2.6 |
| (Å) | | | | |
| FSC threshold | 0.5 | 0.5 | 0.5 | 0.5 |
| Model resolution | 2.1-33.5 | 2.1-37.6 | 2.1-37.0 | 2.0-40.3 |
| range (Å) | | | | |
| Map sharpening | −100 | −100 | −100 | −100 |
| B factor (Å²) | | | | |
| Model | | | | |
| composition | | | | |
| Non- | 15990 | 15994 | 9150 | 9148 |
| hydrogen atoms | | | | |
| Protein | 1960 | 1962 | 1204 | 1206 |
| residues | | | | |
| Ligands | PX4 (8) | PX4 (8) | 0 | 0 |
| R.m.s. deviations | | | | |
| Bond lengths | 0.012 | 0.003 | 0.012 | 0.005 |
| (Å) | | | | |
| Bond angles | 0.810 | 0.675 | 0.937 | 0.719 |
| (°) | | | | |
| Validation | | | | |
| Molprobity | 1.66 | 1.43 | 1.59 | 1.41 |
| score | | | | |
| Clashscore | 5.27 | 3.13 | 4.28 | 4.37 |
| Poor | 0.57 | 0.11 | 0.59 | 0.19 |
| rotamers (%) | | | | |
| Ramachandran | | | | |
| plot | | | | |
| Favored (%) | 94.39 | 95.27 | 94.44 | 95.13 |
| Allowed (%) | 5.30 | 4.62 | 5.22 | 4.37 |
| Disallowed | 0.31 | 0.10 | 0.34 | 0.50 |
| (%) | | | | |
| Rama-Z score | | | | |
| Whole | −1.45 | −1.36 | −0.64 | −0.21 |
| Helix | −0.89 | −1.06 | −0.93 | −1.08 |
| Sheet | −0.58 | −0.15 | 0.25 | 0.75 |
| Loop | −0.98 | −0.77 | −0.56 | −0.22 |

### Ang2

Ang2 was incubated with an equimolar amount of the 5A12.6DS Fab and a 2-fold molar excess of ProA-ProG fusion protein on ice for 30 min. This mixture was injected onto a Superdex 200 3.2/300 column equilibrated in 20 mM HEPES pH 7.5, 150 mM NaCl. The peak fraction was subjected to mild crosslinking with 0.5 mM BS3 at room temperature for 10 min. The crosslinking reaction was quenched by addition of 100 mM Tris pH 7.5. 4 μL of this sample were then applied to glow-discharged Quantifoil R0.6/1 Au300 holey carbon grids, blotted in a Vitrobot MarkIV at 4 °C and 100% humidity using a blotting time of 6 s and a blot force of 7, and plunge-frozen in liquid ethane cooled by liquid nitrogen.

Movie stacks were collected using SerialEM (Mastronarde et al, J Struct Biol, 152:36-51, 2005) on a Titan Krios operated at 300 kV and equipped with a BioQuantum energy filter operated with a 20 eV energy slit with a K3 Summit direct electron detector camera. Images were recorded in EFTEM mode at a magnification of 105,000× corresponding to 0.838 Å per pixel, using a 20 eV energy slit. Each image stack contains 119 frames recorded every 0.05 s for an accumulated dose of 69 e/Å² and a total exposure time of 6 s. Images were recorded with a set defocus range of 0.5 to 1.5 μm.

All image processing was performed using CryoSPARC as summarized in the schematic in FIG. 9. Patch motion correction, patch CTF estimation, and particle picking were performed using CryoSPARC Live. Micrographs with CTF fit resolutions worse than 3.7 Å or with relative ice thickness higher than 1.077 were rejected. The blob picker was used with a minimum radius of 50 Å and maximum radius of 150 Å, using both a circular blob and elliptical blob, as well as a minimum separation distance of 0.3 diameters. Particles were extracted with a box size of 400 px and binned to 128 px. 2D classification of a small subset of the data was performed to identify 2D classes for generation of initial model via ab initio reconstruction. This model was used to generate templates for particle picking with a radius of 100 Å. The resulting particle stack was subjected to 2D classification to remove junk particles. The remaining particles were subjected to iterative rounds of multi-class ab initio reconstruction and heterogeneous refinement, resulting in a stack of 1,017,611 particles, which were re-extracted with a box size of 400 px and binned to 324 px. These particles were then subjected to non-uniform refinement, resulting in a 3D reconstruction with a GS-FSC resolution of 2.69 Å. A local resolution map was calculated using the Local Resolution Estimation job in CryoSPARC.

The crystal structure of the Ang2-5 Å¹².WT complex (PDB: 4ZFG) (Koenig et al, J Biol Chem, 290: 21773-21786, 2015) was used as an initial model to dock into the cryo-EM map in ChimeraX. The resulting model was rebuilt and refined using COOT, ISOLDE, and Phenix. Sharpened maps were generated using the Cryo-EM module in COOT. Figures were generated using ChimeraX and PyMOL.

### KRAS^{G12C}

KRAS^{G12C} was covalently modified with GNE-1952 as previously described in Davies et al, Nat Biotechnol, 40:769-778, 2022). KRAS^{G12C} was incubated for 4 hrs at room temperature with 150 μM GNE-1952, 5 mM GDP, and 20 mM EDTA. Complete covalent modification was confirmed via mass spectrometry. The modified protein was then buffer exchanged into 20 mM HEPES pH 7.0, 100 mM NaCl, 1 mM MgCl₂, 5 μM GDP through size exclusion chromatography on a Superdex 75 16/60 column. The KRAS^{G12C}-GNE-1952 adduct was incubated with a 2-fold molar excess of the 2H11.4DS Fab on ice for 30 min. This mixture was then injected onto a Superdex 200 3.2/300 column equilibrated in 20 mM HEPES pH 7.0, 100 mM NaCl, 1 mM MgCl₂, 5 μM GDP. The peak fraction was subjected to mild crosslinking with 0.5 mM BS3 at room temperature for 10 min. 4 μL of this sample were then applied to glow-discharged Quantifoil R0.6/1 Au300 holey carbon grids, blotted in a Vitrobot MarkIV at 4 °C and 100% humidity using a blotting time of 6 s and a blot force of 7, and plunge-frozen in liquid ethane cooled by liquid nitrogen.

Movie stacks were collected using EPU on a Titan Krios operated at 300 kV and equipped with a Selectris and a Falcon4 detector. Images were recorded at a magnification of 165,000× corresponding to 0.731 Å per pixel, using a 20 eV energy slit. Each image stack contains 1077 frames recorded every 0.005 s for an accumulated dose of 40 e/Å² and a total exposure time of 2.2 s. Images were recorded with a set defocus range of 0.5 to 1.5 μm.

All image processing was performed using CryoSPARC as summarized in the schematic in FIG. 12. Patch motion correction, patch CTF estimation, and particle picking were performed using CryoSPARC Live. Micrographs with CTF fit resolutions worse than 4.0 Å or with relative ice thickness higher than 1.12 were rejected. The blob picker was used with a minimum radius of 50 Å and maximum radius of 150 Å, using both a circular blob and elliptical blob, as well as a minimum separation distance of 0.5 diameters. Particles were extracted with a box size of 324 px and binned to 128 px. 2D classification of a small subset of the data was performed to identify 2D classes for generation of initial model via ab initio reconstruction. This model was used to generate templates for particle picking with a radius of 150 Å. The resulting particle stack was subjected to 2D classification to remove junk particles. The remaining particles were subjected to iterative rounds of multi-class ab initio reconstruction and heterogeneous refinement, resulting in a stack of 926,738 particles, which were re-extracted with a box size of 324 px and binned to 224 px. These particles were then subjected to non-uniform refinement, including refinement of per-particle defocus and per-group CTF parameters (tilt and trefoil), resulting in a 3D reconstruction with a GS-FSC resolution of 2.83 Å. Local refinement was then performed using a mask around KRAS and the 2H11.4DS variable domain, leading to a 3D reconstruction with a GS-FSC resolution of 2.74 Å. Local resolution maps were calculated using the Local Resolution Estimation job in CryoSPARC.

The crystal structure of the KRAS^{G12C}-GNE-1952-2H11.WT complex (PDB: 7RP3; see, Maun et al, Nat Commun, 11:6435, 2020) was used as an initial model to dock into the cryo-EM maps in ChimeraX. The resulting model was rebuilt and refined using COOT, ISOLDE, and Phenix. Sharpened maps were generated using the Cryo-EM module in COOT. Figures were generated using ChimeraX and PyMOL. Cryo-EM data collection, refinement, and validation statistics for Ang2 and KRAS complexes are shown in Table 5. Table 5. Cryo-EM data collection, refinement, and validation statistics for Ang2 and KRAS complexes.

TABLE 5
Cryo-EM data collection, refinement, and validation
statistics for Ang2 and KRAS complexes.

| | Ang2-5A12.6DS | KRAS-2H11.4DS |
|---|---|---|
| EMDB ID | EMD-43220 | EMD-43221 |
| PDB ID | 8VGP | 8VGQ |
| Data collection and processing | | |
| Magnification | 105,000 | 165,000 |
| Voltage (kV) | 300 | 300 |
| Detector | K3 | Falcon 4 |
| Electron exposure (e⁻/Å) | 68.5 | 40.3 |
| Defocus range (μm) | 0.5-1.5 | 0.8-1.8 |
| Pixel size (micrographs) (Å) | 0.838 | 0.731 |
| Symmetry imposed | C1 | C1 |
| Initial particle images (no.) | 9,288,470 | 6,202,602 |
| Final particle images (no.) | 1,017,611 | 926,738 |
| Pixel size (map) (Å) | 1.0346 | 1.0573 |
| Map resolution (Å) | 2.7 | 2.8 |
| FSC threshold | 0.143 | 0.143 |
| Map resolution range (Å) | 2.3-41.0 | 2.4-39.9 |
| Refinement | | |
| Initial model used (PDB code) | 4ZFG | 7RP3 |
| Model resolution (Å) | 2.7 | 2.8 |
| FSC threshold | 0.5 | 0.5 |
| Model resolution range (Å) | 2.3-41.0 | 2.4-39.9 |
| Map sharpening B factor (Å²) | −100 | −100 |
| Model composition | | |
| Non-hydrogen atoms | 5052 | 4672 |
| Protein residues | 648 | 600 |
| Ligands | 0 | MG (1), MKZ (1), |
| | | GDP (1) |
| R.m.s. deviations | | |
| Bond lengths (Å) | 0.004 | 0.005 |
| Bond angles (°) | 0.703 | 0.849 |
| Validation | | |
| Molprobity score | 1.32 | 1.33 |
| Clashscore | 1.94 | 1.32 |
| Poor rotamers (%) | 0.91 | 2.14 |
| Ramachandran plot | | |
| Favored (%) | 94.69 | 96.62 |
| Allowed (%) | 5.16 | 3.04 |
| Disallowed (%) | 0.16 | 0.34 |
| Rama-Z score | | |
| Whole | −0.77 | −0.31 |
| Helix | −2.85 | −1.97 |
| Sheet | 0.45 | 1.48 |
| Loop | −1.00 | −1.06 |

Table 6 below provides an overview of the exemplary antibodies described in the above Example and used to illustrate certain embodiments of the technology disclosed herein.

TABLE 6
Summary of Rigid Fab Sequences

| SEQ | | |
|---|---|---|
| ID | Antibody | |
| NO | Reference | Sequence |
| 1 | anti- | DIQMTQSPSSLSASVGDRVTITCQSIKSVYNNRLGWYQ |
| | Tryptase | QKPGKAPKLLIYETSILTSGVPSRFSGSGSGTDFTLTISS |
| | E104.v1. | LQPEDFATYYCAGGFDRSGDTTFGQGTKVEIKRTVAAP |
| | WT VL | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| 2 | anti- | EVQLVESGPGLVKPSETLSLTCTVSRFSLIGYAITWIRQP |
| | Tryptase | PGKGLEWIGGISSAATTFYSSWAKSRVTISVDTSKNQFS |
| | E104.v1. | LKLSSVTAADTAVYYCARDPRGYGAALDRLDLWGQGTL |
| | WT VH | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHT |
| 3 | anti- | DIQMTQSPSSLSASVGDRVTITCQSIKSVYNNRLGWYQQ |
| | Tryptase | KPGKAPKLLIYETSILTSGVPSRFSGSGSGTDFTLTISSLQ |
| | E104.v1.2 | CEDFATYYCAGGFDRSGDTTFGQGTKVEIKRTVAAPSVF |
| | DS VL | IFPPSDEQLKSGTASVVCLLNNFYPREAKVQWKVDNALQ |
| | | SGNSQESVTEQDSKDCTYSLSSTLTLSKADYEKHKVYAC |
| | | EVTHQGLSSPVTKSFNRGEC |
| 4 | anti- | EVQLVESGPGCVKPSETLSLTCTVSRFSLIGYAITWIRQPP |
| | Tryptase | GKGLEWIGGISSAATTFYSSWAKSRVTISVDTSKNQFSLKL |
| | E104.v1.2 | SSVTAADTAVYYCARDPRGYGAALDRLDLWGQGTLVTVS |
| | DS VH | SASTKGPSVFPLAPSSKSTSGGTAALGCLVKDYFCEPVTV |
| | | SWNSGALTSGVHTFPAVLQSSGLYSLSSVVTVPSSSLGTQ |
| | | TYICNVNHKPSNTKVDKKVEPKSCDKTHTHHHHHHP |
| 5 | anti- | DIQMTQSPSSLSASVGDRVTITCQSIKSVYNNRLGWYQQ |
| | Tryptase | KCGKAPKLLIYETSILTSGVPSRFSGSGSGTDFTLTISSLQ |
| | E104.v1.4 | CEDFATYYCAGGFDRSGDTTFGQGTKVEIKRTVAAPSVF |
| | DS VL | IFPPSDEQLKSGTASVVCLLNNFYPREAKVQWKVDNALQ |
| | | SGNSQESVTCQDSKDCTYSLSSTLTLSKADYEKHKVYAC |
| | | EVTHQGLSSPVTKSFNRGEC |
| 6 | anti- | EVQLVESGPGCVKPSETLSLTCTVSRFSLIGYAITWIRQPP |
| | Tryptase | GKGLEWIGGISSAATTFYSSWAKSRVTISVDTSKNQFSLKL |
| | E104.v1.4 | SSVTAADTAVYYCARDPRGYGAALDRLDLWGQGTCVTVS |
| | DS VH | SASTKGPSVFPLAPSSKSTSGGTAALGCLVKDYFCECPVT |
| | | VSWNSGALTSGVHTFPAVLQSSGLYSLSSVVTVPSSSLGT |
| | | QTYICNVNHKPSNTKVDKKVEPKSCDKTHTHHHHHHP |
| 7 | anti- | DIQMTQSPSSLSASVGDRVTITCQSIKSVYNNRLGWYQ |
| | Tryptase | QKCGKAPKLLIYETSILTSGVPSRFSGSGSGTDFTLTISS |
| | E104.v1.5 | LQCEDFATYYCAGGFDRSGDTTFGQGTKVEIKRTVAAP |
| | DS VL | SVCIFPPSDEQLKSGTASVVCLLNNFYPREAKVQWKVD |
| | | NALQSGNSQESVTCQDSKDCTYSLSSTLTLSKADYEKH |
| | | KVYACEVTHQGLSSPVTKSFNRGEC |
| 8 | anti- | EVQLVESGPGCVKPSETLSLTCTVSRFSLIGYAITWIRQP |
| | Tryptase | PGKGLEWIGGISSAATTFYSSWAKSRVTISVDTSKNQFS |
| | E104.v1.5 | LKLSSVTAADTAVYYCARDPRGYGAALDRLDLWGQGTC |
| | DS | VTVSSASTKGPSVFPLAPSSKSTSGGTACLGCLVKDYFC |
| | VH | ECPVTVSWNSGALTSGVHTFPAVLQSSGLYSLSSVVTVP |
| | | SSSLGTQTYICNVNHKPSNTKVDKKVEPKSCDKTHTHHH |
| | | HHHP |
| 9 | anti- | DIQMTQSPSSLSASVGDRVTITCQSIKSVYNNRLGWYQQ |
| | Tryptase | KCGKAPKLLIYETSILTSGVPSRFSGSGSGTDFTLTISSLQ |
| | E104.v1.6 | CEDFATYYCAGGFDRSGDTTFGQGTKVEIKRTVAAPSVC |
| | DS VL | IFPPSDECLKSGTASVVCLLNNFYPREAKVQWKVDNALQ |
| | | SGNSQESVTCQDSKDCTYSLSSTLTLSKADYEKHKVYAC |
| | | EVTHQGLSSPVTKSFNRGEC |
| 10 | anti- | EVQLVESGPGCVKPSETLSLTCTVSRFSLIGYAITWIRQPP |
| | Tryptase | GKGLEWIGGISSAATTFYSSWAKSRVTISVDTSKNQFSLK |
| | E104.v1.6 | LSSVTAADTAVYYCARDPRGYGAALDRLDLWGQGTCVT |
| | DS VH | VSSASTKGPSVCPLAPSSKSTSGGTACLGCLVKDYFCEC |
| | | PVTVSWNSGALTSGVHTFPAVLQSSGLYSLSSVVTVPSS |
| | | SLGTQTYICNVNHKPSNTKVDKKVEPKSCDKTHTHHHHH |
| | | HP |
| 11 | anti- | EIVLTQSPALMAASPGEKVTITCSVSLSISSSNLFWYQQKS |
| | Nav1.7 | ETSPKPWIYGTSKLASGVPVRFSGSGSGTSYSLTISSMEA |
| | 7A9.WT | EDAATYYCQQWSSHSFTFGGGTKLEIKRADAAPTVSIFPP |
| | VL | SSEQLTSGGASVVCFLNNFYPKDINVKWKIDGSERQNGVL |
| | | NSWTDQDSKDSTYSMSSTLTLTKDEYERHNSYTCEATHKT |
| | | STSPIVKSFNRNEC |
| 12 | anti- | EVQLVESGGGLVKPGGSLKLSCAASGFTFSNYAMSWVRQ |
| | Nav1.7 | TPEKRLEWVATISNGGRYTYYPDSVKGRFTISRDNAKNSLY |
| | 7A9.WT | LQMSSLRSEDTAMYYCARHLYRYDVGGALDYWGQGTSVT |
| | VH | VSSAKTTAPSVYPLAPVCGDTTGSSVTLGCLVKGYFPEPVT |
| | | LTWNSGSLSSGVHTFPAVLQSDLYTLSSSVTVTSSTWPSQS |
| | | ITCNVAHPASSTKVDKKIEPRGPTIKP |
| 13 | anti- | EIVLTQSPALMAASPGEKVTITCSVSLSISSSNLFWYQQKC |
| | Nav1.7 | ETSPKPWIYGTSKLASGVPVRFSGSGSGTSYSLTISSMEC |
| | 7A9.4DS | EDAATYYCQQWSSHSFTFGGGTKLEIKRADAAPTVSIFPP |
| | VL | SSEQLTSGGASVVCFLNNFYPKDINVKWKIDGSERQNGVL |
| | | NSWTCQDSKDCTYSMSSTLTLTKDEYERHNSYTCEATHK |
| | | TSTSPIVKSFNRNEC |
| 14 | anti- | EVQLVESGGGCVKPGGSLKLSCAASGFTFSNYAMSWVRQ |
| | Nav1.7 | TPEKRLEWVATISNGGRYTYYPDSVKGRFTISRDNAKNSLY |
| | 7A9.4DS | LQMSSLRSEDTAMYYCARHLYRYDVGGALDYWGQGTCVT |
| | VH | VSSAKTTAPSVYPLAPVCGDTTGSSVTLGCLVKGYFCECPV |
| | | TLTWNSGSLSSGVHTFPAVLQSDLYTLSSSVTVTSSTWPSQ |
| | | SITCNVAHPASSTKVDKKIEPRGPTIKPHHHHHHP |
| 15 | anti-CD20 | QIVLSQSPAILSASPGEKVTMTCRASSSVSYIHWFQQKPGSS |
| | RTX.WT | PKPWIYATSNLASGVPVRFSGSGSGTSYSLTISRVEAEDAAT |
| | VL | YYCQQWTSNPPTFGGGTKLEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 16 | anti-CD20 | QVQLQQPGAELVKPGASVKMSCKASGYTFTSYNMHWVKQ |
| | RTX.WT | TPGRGLEWIGAIYPGNGDTSYNQKFKGKATLTADKSSSTAY |
| | VH | MQLSSLTSEDSAVYYCARSTYYGGDWYFNVWGAGTTVTV |
| | | SAASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKKVEPKSCDKTHT |
| 17 | anti-CD20 | QIVLSQSPAILSASPGEKVTMTCRASSSVSYIHWFQQKCGSSP |
| | RTX.4DS | KPWIYATSNLASGVPVRESGSGSGTSYSLTISRVECEDAATY |
| | VL | YCQQWTSNPPTFGGGTKLEIKRTVAAPSVFIFPPSDEQLKSG |
| | | TASVVCLLNNFYPREAKVQWKVDNALQSGNSQESVTCQDS |
| | | KDCTYSLSSTLTLSKADYEKHKVYACEVTHQGLSSPVTKSF |
| | | NRGEC |
| 18 | anti-CD20 | QVQLQQPGAECVKPGASVKMSCKASGYTFTSYNMHWVK |
| | RTX.4DS | QTPGRGLEWIGAIYPGNGDTSYNQKFKGKATLTADKSSSTA |
| | VH | YMQLSSLTSEDSAVYYCARSTYYGGDWYFNVWGAGTCVT |
| | | VSAASTKGPSVFPLAPSSKSTSGGTAALGCLVKDYFCECPVT |
| | | VSWNSGALTSGVHTFPAVLQSSGLYSLSSVVTVPSSSLGTQT |
| | | YICNVNHKPSNTKVDKKVEPKSCDKTHTHHHHHHP |
| 19 | anti-Ang2 | DIQMTQSPSSLSASVGDRVTITCRASQFLSSFGVAWYQQKPG |
| | 5A12.WT | KAPKLLIYGASSLYSGVPSRFSGSGSGTDFTLTISSLQPEDFA |
| | VL | TYYCQQGLLSPLTFGQGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 20 | anti-Ang2 | EVQLVESGGGLVQPGGSLRLSCAASGFTISDYWIHWVRQAP |
| | 5A12.WT | GKGLEWVAGITPAGGYTYYADSVKGRFTISADTSKNTAYLQ |
| | VH | MNSLRAEDTAVYYCARFVFFLPYAMDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHT |
| 21 | anti-Ang2 | DIQMTQSPSSLSASVGDRVTITCRASQFLSSFGVAWYQQK |
| | 5A12.6DS | CGKAPKLLIYGASSLYSGVPSRFSGSGSGTDFTLTISSLQCE |
| | VL | DFATYYCQQGLLSPLTFGQGTKVEIKRTVAAPSVCIFPPSDE |
| | | CLKSGTASVVCLLNNFYPREAKVQWKVDNALQSGNSQES |
| | | VTCQDSKDCTYSLSSTLTLSKADYEKHKVYACEVTHQGLS |
| | | SPVTKSFNRGEC |
| 22 | anti-Ang2 | EVQLVESGGGCVQPGGSLRLSCAASGFTISDYWIHWVRQ |
| | 5A12.6DS | APGKGLEWVAGITPAGGYTYYADSVKGRFTISADTSKNT |
| | VH | AYLQMNSLRAEDTAVYYCARFVFFLPYAMDYWGQGTC |
| | | VTVSSASTKGPSVCPLAPSSKSTSGGTACLGCLVKDYFCE |
| | | CPVTVSWNSGALTSGVHTFPAVLQSSGLYSLSSVVTVPSS |
| | | SLGTQTYICNVNHKPSNTKVDKKVEPKSCDKTHTHHHHHH |
| | | P |
| 23 | anti- | SVLTQPPSASGTPGQRVTISCSGSSSNIGSNYVYWYQQLPGT |
| | KRAS | APKLLIYRNNQRPSGVPDRFSGSKSGTSASLAISGLRSEDEAD |
| | 2H11.WT | YYCAAWDERLSGWVFGGGTKLTVLGQPKAAPSVTLFPPSSE |
| | VL | ELQANKATLVCLISDFYPGAVTVAWKADSSPVKAGVETTTP |
| | | SKQSNNKYAASSYLSLTPEQWKSHRSYSCQVTHEGSTVEKT |
| | | VAPTECS |
| 24 | anti- | EVQLQESGPGLVKPPGTLSLTCAVSGGSISSSNWWSWVRQP |
| | KRAS | PGKGLEWIGEIYHSGSTNYNPSLKSRVTISVDKSKNQFSLKL |
| | 2H11.WT | SSVTAADTAVYYCARGSSSWYDLGPFDYWGQGTLVTVSSA |
| | VH | STKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKKVEPKSCDTHT |
| 25 | anti- | SVLTQPPSASGTPGQRVTISCSGSSSNIGSNYVYWYQQLCGT |
| | KRAS | APKLLIYRNNQRPSGVPDRFSGSKSGTSASLAISGLRCEDEA |
| | 2H11.4DS | DYYCAAWDERLSGWVFGGGTKLTVLGQPKAAPSVTLFPPS |
| | VL | SEELQANKATLVCLISDFYPGAVTVAWKADSSPVKAGVETT |
| | | TPSCQSNCNKYAASSYLSLTPEQWKSHRSYSCQVTHEGSTV |
| | | EKTVAPTECS |
| 26 | anti- | EVQLQESGPGCVKPPGTLSLTCAVSGGSISSSNWWSWVRQP |
| | KRAS | PGKGLEWIGEIYHSGSTNYNPSLKSRVTISVDKSKNQFSLKL |
| | 2H11.4DS | SSVTAADTAVYYCARGSSSWYDLGPFDYWGQGTCVTVSSA |
| | VH | STKGPSVFPLAPSSKSTSGGTAALGCLVKDYFCECPVTVSW |
| | | NSGALTSGVHTFPAVLQSSGLYSLSSVVTVPSSSLGTQTYIC |
| | | NVNHKPSNTKVDKKVEPKSCDKTHTHHHHHHP |

The positions at which substitution or insertion was made to allow formation of a disulfide bond are shown in bold and underlined.

What is claimed is:

1. A method of determining a high-resolution structure of a molecule comprising determining a high-resolution structure of a complex comprising the molecule and an antibody or antigen-binding fragment thereof or an antibody scaffold that binds to the molecule,
wherein the antibody or antigen-binding fragment or the antibody scaffold comprises one or more engineered disulfide bonds that increase conformational rigidity of the antibody or antigen-binding fragment thereof or the antibody scaffold,
and thereby determining the high-resolution structure of the molecule.

2. The method of claim 1, wherein the complex comprising the molecule and the antibody or antigen-binding fragment thereof or the antibody scaffold is a non-covalent complex.

3. The method of claim 1 or 2, wherein the complex comprising the molecule and the antibody or antigen-binding fragment thereof or the antibody scaffold is at least 40 kDa.

4. The method of any one of claims 1-3, wherein the antibody or antigen-binding fragment thereof or the antibody scaffold comprises a variable domain and a constant domain, and
wherein the one or more engineered disulfide bonds are between a cysteine located in the variable domain and a cysteine located in the constant domain.

5. A method of increasing the rigidity of an antibody or antigen-binding fragment thereof or an antibody scaffold comprising a constant domain and a variable domain, the method comprising
introducing a cysteine residue in the constant domain and introducing a cysteine residue in the variable domain, wherein the cysteine residue in the constant domain and the cysteine residue in the variable domain are sufficiently close in at least one conformation of the antibody or antigen-binding fragment thereof or the antibody scaffold to form an engineered disulfide bond, and
allowing or inducing formation of the engineered disulfide bond, thereby increasing the rigidity of the antibody or antigen-binding fragment thereof or the antibody scaffold.

6. The method of claim 4 or 5, wherein the one or more engineered disulfide bonds reduce the variability in the elbow angle between the variable domain and the constant domain of the antibody or antigen-binding fragment thereof or the antibody scaffold.

7. The method of claim 6, wherein the elbow angle of the antibody or antigen-binding fragment thereof or the antibody scaffold is between 137 and 159 degrees.

8. The method of claim 6 or 7, wherein the elbow angle range is 5 degrees or less.

9. The method of any one of claims 1-8, wherein each of the one or more engineered disulfide bonds is between two amino acids that are positioned within 5.5 Å of each other in at least one conformation of the antibody or antigen-binding fragment thereof or the antibody scaffold.

10. The method of claim 9, wherein each of the one or more engineered disulfide bonds is between two amino acids whose C-beta atoms are positioned within 5.5 Å of each other in at least one conformation of the antibody or antigen-binding fragment thereof or the antibody scaffold.

11. The method of any one of claims 1-10, wherein the one or more engineered disulfide bonds are two, three, four, five, or six engineered disulfide bonds.

12. The method of any one of claims 1-11, wherein the one or more engineered disulfide bonds comprise one disulfide bond between the heavy chain constant domain and the heavy chain variable domain and one disulfide bond between the light chain constant domain and the light chain variable domain.

13. The method of any one of claims 1-12, wherein the one or more engineered disulfide bonds comprise two disulfide bonds between the heavy chain constant domain and the heavy chain variable domain and two disulfide bonds between the light chain constant domain and the light chain variable domain.

14. The method of any one of claims 1-13, wherein the one or more engineered disulfide bonds comprise one or more disulfide bonds between the light chain constant domain and the heavy chain constant domain.

15. The method of any one of claims 1-14, wherein the antibody or antigen-binding fragment thereof or the antibody scaffold is murine, rabbit, chimeric, humanized or human.

16. The method of any one of claims 1-4 or 6-15, wherein the resolution of the high-resolution structure of the complex and/or the high-resolution structure of the molecule is determined to better than 4 Å, better than 3.5 Å, better than 3 Å or better than 2.8 Å.

17. The method of any one of claims 1-16, wherein the one or more engineered disulfide bonds comprise residue pairs, according to Kabat numbering, 11^{HC}:151^{HC}, 108^{HC}:152^{HC}_153^{HC}insC, 40^{LC}:165^{LC}, 40^{LC}:166^{LC}, 80^{LC}:171^{LC}, 141^{HC}:116^{LC}, 126^{HC}:124^{LC}, 80^{LC}:170^{LC}_171^{LC}insC, 110^{HC}:151^{HC}, 106^{LC}:171^{LC}, 83^{LC}:166^{LC}, 81^{LC}:168^{LC}, 14^{HC}:113^{HC}, 14^{HC}:107^{HC}, 45^{HC}:44^{LC}, 183^{HC}:176^{LC} and/or 128^{HC}:118^{LC}.

18. The method of claim 17, wherein the one or more engineered disulfide bonds comprise 11^{HC}:151^{HC} and 80^{LC}:171^{LC}.

19. The method of claim 17, wherein the one or more engineered disulfide bonds comprise 11^{HC}:151^{HC}, 108^{HC}:152^{HC}_153^{HC}insC, and 40^{LC}:165^{LC}, and 80^{LC}:171^{LC}.

20. The method of claim 17, wherein the one or more engineered disulfide bonds comprise 11^{HC}:151^{HC}, 108^{HC}:152^{HC}_153^{HC}insC, 40^{LC}:165^{LC}, 80^{LC}:171^{LC} and 141^{HC}:116^{LC}.

21. The method of claim 17, wherein the one or more engineered disulfide bonds comprise 11^{HC}:151^{HC}, 108^{HC}:152^{HC}_153^{HC}insC, 40^{LC}:165^{LC}, 80^{LC}:171^{LC}, 141^{HC}:116^{LC} and 126^{HC}: 124^{LC}.

22. The method of claim 17, wherein the one or more engineered disulfide bonds comprise 11^{HC}:151^{HC}, 108^{HC}:152^{HC}_153^{HC}insC, 40^{LC}:166^{LC} and 80^{LC}:170^{LC}_171^{LC}insC.

23. The method of claim 17, wherein the one or more engineered disulfide bonds comprise 11^{HC}:151^{HC}, 108^{HC}:152^{HC}_153^{HC}insC, 40^{LC}:166^{LC}, 80^{LC}: 17^{LC}_17^{LC}insC, 141^{HC}:116^{LC}, and 126^{HC}:124^{LC}.

24. The method of any one of claims 1-23, wherein the antibody or antigen-binding fragment thereof or the antibody scaffold comprises a kappa light chain or a lambda light chain.

25. The method of any one of claims 17-21, wherein the antibody or antigen-binding fragment thereof or the antibody scaffold comprises a kappa light chain.

26. The method of any one of claims 17 or 22-23, wherein the antibody or antigen-binding fragment thereof or the antibody scaffold comprises a lambda light chain.

27. The method of any one of claims 1-26, wherein the molecule is a small molecule, peptide, protein, fusion protein, nucleic acid, or a lipid.

28. The method of any one of claims 1-27, wherein the molecule is a cytokine, a drug, or an enzyme substrate.

29. The method of any one of claims 1-28, wherein the molecule is less than 50 kDa, less than 30 kDa, or less than 10 kDa.

30. The method of any one of claims 1-4 or 6-29, wherein determining the high-resolution structure comprises performing cryo-electron microscopy (cryo-EM) on the complex.

31. The method of claim 30, further comprising processing cryo-EM images.

32. The method of claim 30 or 31, further comprising performing refinement of an initial structure.

33. The method of any one of claims 30-32, further comprising performing validation of a structure to determine the high-resolution structure.

34. The method of any one of claims 30-33, further comprising generating the antibody or antigen-binding fragment thereof or the antibody scaffold that binds to the molecule, prior to determining the high-resolution structure.

35. The method of any one of claims 30-34, further comprising incubating the antibody or antigen-binding fragment thereof or the antibody scaffold with the molecule to form a complex, prior to determining the high-resolution structure.

36. The method of claim 35, further comprising purifying the complex after the incubation.

37. The method of any one of claims 30-36, further comprising, prior to determining the high-resolution structure, producing the antibody or antigen-binding fragment thereof or the antibody scaffold comprising one or more engineered disulfide bonds that increase rigidity of the antibody by
(i) analyzing structural data from the antibody or antigen-binding fragment thereof or the antibody scaffold, or from an antibody or antigen-binding fragment thereof or the antibody scaffold obtained from the same species as the antibody or antigen-binding fragment thereof or the antibody scaffold;
(ii) identifying amino acid residue positions in the constant domain and the variable domain that are sufficiently close to form a disulfide bond; and
(iii) introducing a cysteine at the identified amino acid residue positions or inserting a cysteine before or after the identified amino acid residue positions.

38. The method of any one of claims 1-37, wherein the method is performed in vitro.

39. The method of any one of claims 1-38, wherein the antibody or antigen-binding fragment thereof or the antibody scaffold is an antibody.

40. The method of any one of claims 1-38, wherein the antibody or antigen-binding fragment thereof or the antibody scaffold is an antigen-binding antibody fragment.

41. The method of claim 40, wherein the antigen-binding antibody fragment is a Fab.

42. The method of any one of claims 1-38, wherein the antibody or antigen-binding fragment thereof or the antibody scaffold is an antibody scaffold.

43. An antibody or antigen-binding fragment thereof or an antibody scaffold comprising one or more engineered disulfide bonds that increase rigidity of the antibody or antigen-binding fragment thereof or the antibody scaffold.

44. The antibody or antigen-binding fragment thereof or the antibody scaffold of claim 43,
wherein the antibody or antigen-binding fragment thereof or the antibody scaffold comprises a variable domain and a constant domain, and
wherein the one or more engineered disulfide bonds are between a cysteine located in the variable domain and a cysteine located in the constant domain.

45. The antibody or antigen-binding fragment thereof or the antibody scaffold of claims 43 or 44, wherein the one or more engineered disulfide bonds reduce the variability in the elbow angle between the variable domain and the constant domain of the antibody or antigen-binding fragment thereof or the antibody scaffold.

46. The antibody or antigen-binding fragment thereof or the antibody scaffold of any one of claims 43-45, wherein the one or more engineered disulfide bonds are between two amino acids that are positioned within 5.5 Å of each other in at least one conformation.

47. The antibody or antigen-binding fragment thereof or the antibody scaffold of any one of claims 43-46, wherein the one or more engineered disulfide bonds are two, three, four, five, or six engineered disulfide bonds.

48. The antibody or antigen-binding fragment thereof or the antibody scaffold of any one of claims 43-47, wherein the one or more engineered disulfide bonds are comprised of one disulfide bond between the heavy chain constant domain and one disulfide bond between the light chain constant domain and the light chain variable domain.

49. The antibody or antigen-binding fragment thereof or the antibody scaffold of any one of claims 43-48, wherein the one or more engineered disulfides are comprised of two disulfide bonds between the heavy chain constant domain and two disulfide bonds between the light chain constant domain and the light chain variable domain.

50. The antibody or antigen-binding fragment thereof or the antibody scaffold of any one of claims 43-49, wherein the one or more engineered disulfide bonds comprises one or more disulfide bonds between the light chain constant domain and the heavy chain constant domain.

51. The antibody or antigen-binding fragment thereof or the antibody scaffold of any one of claims 43-50, wherein the one or more engineered disulfide bonds comprise residue pairs, according to Kabat numbering, 11^{HC}:151^{HC}, 108^{HC}:152^{HC}_153^{HC}insC, 40^{LC}:165^{LC}, 40^{LC}:166^{LC}, 80^{LC}:171^{LC}, 141^{HC}:116^{LC}, 126^{HC}:124^{LC}, 80^{LC}:170^{LC}_171^{LC}insC, 110^{HC}:151^{HC}, 106^{LC}:171^{LC}, 83^{LC}:166^{LC}, 81^{LC}:168^{LC}, 14^{HC}:113^{HC}, 14^{HC}:107^{HC}, 45^{HC}:44^{LC}, 183^{HC}:176^{LC} and/or 128^{HC}: 118^{LC}.

52. The antibody or antigen-binding fragment thereof or the antibody scaffold of claim 51, wherein the one or more engineered disulfide bonds comprise 11^{HC}:151^{HC} and 80^{LC}:71^{LC}.

53. The antibody or antigen-binding fragment thereof or the antibody scaffold of claim 51, wherein the one or more engineered disulfide bonds comprise 11^{HC}:151^{HC}, 108^{HC}:152^{HC}_153^{HC}insC, and 40^{LC}:165^{LC} and 80^{LC}:171^{LC}.

54. The antibody or antigen-binding fragment thereof or the antibody scaffold of claim 51, wherein the one or more engineered disulfide bonds comprise 11^{HC}:151^{HC}, 108^{HC}:152^{HC}_153^{HC}insC, 40^{LC}:165^{LC}, 80^{LC}:171^{LC} and 141^{HC}:116^{LC}.

55. The antibody or antigen-binding fragment thereof or the antibody scaffold of claim 51, wherein the one or more engineered disulfide bonds comprise 11^{HC}:151^{HC}, 108^{HC}:152^{HC}_153^{HC}insC, 40^{LC}:165^{LC}, 80^{LC}:171^{LC}, 141^{HC}:116^{LC} and 126^{HC}:124^{LC}.

56. The antibody or antigen-binding fragment thereof or the antibody scaffold of claim 51, wherein the one or more engineered disulfide bonds comprise 11^{HC}:151^{HC}, 108^{HC}:152^{HC}_153^{HC}insC, 40^{LC}:166^{LC} and 80^{LC}:170^{LC}_71^{LC}insC.

57. The antibody or antigen-binding fragment thereof or the antibody scaffold of claim 51, wherein the one or more engineered disulfide bonds comprise 11^{HC}:151^{HC}, 108^{HC}:152^{HC}_153^{HC}insC, 40^{LC}:166^{LC}, 80^{LC}:170^{LC}_171^{LC}insC, 141^{HC}:116^{LC} and 126^{HC}:124^{LC}.

58. The antibody or antigen-binding fragment thereof or the antibody scaffold of any one of claims 43-57, wherein the antibody or antigen-binding fragment thereof or the antibody scaffold comprises a kappa light chain or a lambda light chain.

59. The antibody or antigen-binding fragment thereof or the antibody scaffold of any one of claims 51-55, wherein the antibody or antigen-binding fragment thereof or the antibody scaffold comprises a kappa light chain.

60. The antibody or antigen-binding fragment thereof or the antibody scaffold of any one of claims 51 or 56-57, wherein the antibody or antigen-binding fragment thereof or the antibody scaffold comprises a lambda light chain.

61. The antibody or antigen-binding fragment thereof or the antibody scaffold of any one of claims 43-60, wherein the antibody or antigen-binding fragment thereof or the antibody scaffold is an antibody.

62. The antibody or antigen-binding fragment thereof or the antibody scaffold of any one of claims 43-60, wherein the antibody or antigen-binding fragment thereof or the antibody scaffold is an antigen-binding antibody fragment.

63. The antibody or antigen-binding fragment thereof or the antibody scaffold of claim 62, wherein the antigen-binding antibody fragment is a Fab.

64. The antibody or antigen-binding fragment thereof or the antibody scaffold of any one of claims 43-60, wherein the antibody or antigen-binding fragment thereof or the antibody scaffold is an antibody scaffold.

65. A complex comprising the antibody or antigen-binding fragment thereof or the antibody scaffold of any one of claims 43-64 and a molecule.

66. The complex of claim 65, wherein the complex comprising the molecule and the antibody or antigen-binding fragment thereof or the antibody scaffold is a non-covalent complex.

67. The complex of claim 65 or 66, wherein the complex comprising the molecule and the antibody or antigen-binding fragment thereof or the antibody scaffold is at least 40 kDa.

68. The complex of any one of claims 65-67, wherein the antibody or antigen-binding fragment thereof or the antibody scaffold is an antibody.

69. The complex of any one of claims 65-67, wherein the antibody or antigen-binding fragment thereof or the antibody scaffold is an antigen-binding fragment of an antibody.

70. The complex of claim 69, wherein the antigen-binding fragment of an antibody is a Fab.

71. The complex of any one of claims 65-67, wherein the antibody or antigen-binding fragment thereof or the antibody scaffold is an antibody scaffold.
